(19) United States
(12) Patent Application Publication
(10) Pub. No.: US 2007/0259860 A1
(43) Pub. Date: Nov. 8, 2007

(54) MGluR5 modulators V

(75) Inventors: Andreas Wallberg, Molndal (SE); Karolina Nilsson, Molndal (SE); Kenneth Granberg, Molndal (SE); Saswati Ghosal, Molndal (SE); Abdelmalik Slassi, Mississauga (CA); Louise Edwards, Mississauga (CA); Methvin Isaac, Brampton (CA); Tao Xin, Woodbridge (CA); Tomislav Stefanac, Burlington (CA); Peter Dove, Toronto (CA)

Correspondence Address: BIRCH, STEWART, KOLASCH & BIRCH, LLP, P.O. BOX 747, 8110 GATEHOUSE ROAD, SUITE 500 EAST, FALLS CHURCH, VA, 22040-0747, US

(73) Assignees: AstraZeneca AB, Sodertalje (SE); NPS PHARMACEUTICALS, INC., Salt Lake City, UT (US)

(21) Appl. No.: 11/790,429

(22) Filed: Apr. 25, 2007

Related U.S. Application Data
(60) Provisional application No. 60/797,663, filed on May 5, 2006.

(51) Int. Cl. A61K 31/5513; A61K 31/519; C07D 487/04

(52) U.S. Cl. 514/221; 514/255.05; 514/259.31; 540/568; 544/263

(57) ABSTRACT

The present invention is directed to novel compounds, to a process for their preparation, their use in therapy and pharmaceutical compositions comprising the novel compounds.

### FIELD OF THE INVENTION

The present invention is directed to novel compounds, their use in therapy and pharmaceutical compositions comprising said novel compounds.

### BACKGROUND OF THE INVENTION

Glutamate is the major excitatory neurotransmitter in the mammalian central nervous system (CNS). Glutamate produces its effects on central neurons by binding to and thereby activating cell surface receptors. These receptors have been divided into two major classes, the ionotropic and metabotropic glutamate receptors, based on the structural features of the receptor proteins, the means by which the receptors transduce signals into the cell, and pharmacological profiles.

The metabotropic glutamate receptors (mGluRs) are G protein-coupled receptors that activate a variety of intracellular second messenger systems following the binding of glutamate. Activation of mGluRs in intact mammalian neurons elicits one or more of the following responses: activation of phospholipase C; increases in phosphoinositide (PI) hydrolysis; intracellular calcium release; activation of phospholipase D; activation or inhibition of adenyl cyclase; increases or decreases in the formation of cyclic adenosine monophosphate (cAMP); activation of guanylyl cyclase; increases in the formation of cyclic guanosine monophosphate (cGMP); activation of phospholipase A₂; increases in arachidonic acid release; and increases or decreases in the activity of voltage- and ligand-gated ion channels. Schoepp et al., Trends Pharmacol. Sci. 14:13 (1993), Schoepp, Neurochem. Int. 24:439 (1994), Pin et al., Neuropharmacology 34:1 (1995), Bordi and Ugolini, Prog. Neurobiol. 59:55 (1999).

Molecular cloning has identified eight distinct mGluR subtypes, termed mGluR1 through mGluR8. Nakanishi, Neuron 13:1031 (1994), Pin et al., Neuropharmacology 34:1 (1995), Knopfel et al., J. Med. Chem. 38:1417 (1995). Further receptor diversity occurs via expression of alternatively spliced forms of certain mGluR subtypes. Pin et al., PNAS 89:10331 (1992), Minakami et al., BBRC 199:1136 (1994), Joly et al., J. Neurosci. 15:3970 (1995).

Metabotropic glutamate receptor subtypes may be subdivided into three groups, Group I, Group II, and Group III mGluRs, based on amino acid sequence homology, the second messenger systems utilized by the receptors, and by their pharmacological characteristics. Group I mGluR comprises mGluR1, mGluR5 and their alternatively spliced variants. The binding of agonists to these receptors results in the activation of phospholipase C and the subsequent mobilization of intracellular calcium.

### Neurological, Psychiatric and Pain Disorders

Attempts at elucidating the physiological roles of Group I mGluRs suggest that activation of these receptors elicits neuronal excitation. Various studies have demonstrated that Group I mGluR agonists can produce postsynaptic excitation upon application to neurons in the hippocampus, cerebral cortex, cerebellum, and thalamus, as well as other CNS regions. Evidence indicates that this excitation is due to direct activation of postsynaptic mGluRs, but it also has been suggested that activation of presynaptic mGluRs occurs, resulting in increased neurotransmitter release. Baskys, Trends Pharmacol. Sci. 15:92 (1992), Schoepp, Neurochem. Int. 24:439 (1994), Pin et al., Neuropharmacology 34:1 (1995), Watkins et al., Trends Pharmacol. Sci. 15:33 (1994).

Metabotropic glutamate receptors have been implicated in a number of normal processes in the mammalian CNS. Activation of mGluRs has been shown to be required for induction of hippocampal long-term potentiation and cerebellar long-term depression. Bashir et al., Nature 363:347 (1993), Bortolotto et al., Nature 368:740 (1994), Aiba et al., Cell 79:365 (1994), Aiba et al., Cell 79:377 (1994). A role for mGluR activation in nociception and analgesia also has been demonstrated, Meller et al., Neuroreport 4: 879 (1993), Bordi and Ugolini, Brain Res. 871:223 (1999). In addition, mGluR activation has been suggested to play a modulatory role in a variety of other normal processes including synaptic transmission, neuronal development, apoptotic neuronal death, synaptic plasticity, spatial learning, olfactory memory, central control of cardiac activity, waking, motor control and control of the vestibulo-ocular reflex. Nakanishi, Neuron 13: 1031 (1994), Pin et al., Neuropharmacology 34: 1, Knopfel et al., J. Med. Chem. 38:1417 (1995).

Further, Group I metabotropic glutamate receptors and mGluR5 in particular, have been suggested to play roles in a variety of pathophysiological processes and disorders affecting the CNS. These include stroke, head trauma, anoxic and ischemic injuries, hypoglycemia, epilepsy, neurodegenerative disorders such as Alzheimer's disease and pain. Schoepp et al., Trends Pharmacol. Sci. 14:13 (1993), Cunningham et al., Life Sci. 54:135 (1994), Hollman et al., Ann. Rev. Neurosci. 17:31 (1994), Pin et al., Neuropharmacology 34:1 (1995), Knopfel et al., J. Med. Chem. 38:1417 (1995), Spooren et al., Trends Pharmacol. Sci. 22:331 (2001), Gasparini et al. Curr. Opin. Pharmacol. 2:43 (2002), Neugebauer Pain 98:1 (2002). Much of the pathology in these conditions is thought to be due to excessive glutamate-induced excitation of CNS neurons. Because Group I mGluRs appear to increase glutamate-mediated neuronal excitation via postsynaptic mechanisms and enhanced presynaptic glutamate release, their activation probably contributes to the pathology. Accordingly, selective antagonists of Group I mGluR receptors could be therapeutically beneficial, specifically as neuroprotective agents, analgesics or anticonvulsants.

Recent advances in the elucidation of the neurophysiological roles of metabotropic glutamate receptors generally and Group I in particular, have established these receptors as promising drug targets in the therapy of acute and chronic neurological and psychiatric disorders and chronic and acute pain disorders.

### Gastrointestinal Disorders

The lower esophageal sphincter (LES) is prone to relaxing intermittently. As a consequence, fluid from the stomach can pass into the esophagus since the mechanical barrier is temporarily lost at such times, an event hereinafter referred to as “reflux”.

Gastro-esophageal reflux disease (GERD) is the most prevalent upper gastrointestinal tract disease. Current pharmacotherapy aims at reducing gastric acid secretion, or at neutralizing acid in the esophagus. The major mechanism behind reflux has been considered to depend on a hypotonic lower esophageal sphincter. However, e.g. Holloway & Dent (1990) Gastroenterol. Clin. N. Amer. 19, pp. 517-535, has shown that most reflux episodes occur during transient lower esophageal sphincter relaxations (TLESRs), i.e. relaxations not triggered by swallows. It has also been shown that gastric acid secretion usually is normal in patients with GERD.

The novel compounds according to the present invention are assumed to be useful for the inhibition of transient lower esophageal sphincter relaxations (TLESRs) and thus for treatment of gastro-esophageal reflux disorder (GERD).

It is well known that certain compounds may cause undesirable effects on cardiac repolarisation in man, observed as a prolongation of the QT interval on electrocardiograms (ECG). In extreme circumstances, this drug-induced prolongation of the QT interval can lead to a type of cardiac arrhythmia called Torsades de Pointes (TdP; Vandenberg et al. hERG K⁺ channels: friend and foe. Trends Pharmacol Sci 2001; 22: 240-246), leading ultimately to ventricular fibrillation and sudden death. The primary event in this syndrome is inhibition of the rapid component of the delayed rectifying potassium current (IKr) by these compounds. The compounds bind to the aperture-forming alpha sub-units of the channel protein carrying this current—sub-units that are encoded by the human ether-a-go-go-related gene (hERG). Since IKr plays a key role in repolarisation of the cardiac action potential, its inhibition slows repolarisation and this is manifested as a prolongation of the QT interval. Whilst QT interval prolongation is not a safety concern per se, it carries a risk of cardiovascular adverse effects and in a small percentage of people it can lead to TdP and degeneration into ventricular fibrillation.

Generally, compounds of the present invention have low activity against the hERG-encoded potassium channel. In this regard, low activity against hERG in vitro is indicative of low activity in vivo.

It is also desirable for drugs to possess good metabolic stability in order to enhance drug efficacy. Stability against human microsomal metabolism in vitro is indicative of stability towards metabolism in vivo.

Because of their physiological and pathophysiological significance, there is a need for new potent mGluR agonists and antagonists that display a high selectivity for mGluR subtypes, particularly the Group I receptor subtype, most particularly the mGluR5.

The object of the present invention is to provide compounds exhibiting an activity at metabotropic glutamate receptors (mGluRs), especially at the mGluR5 receptor. In particular, the compounds according to the present invention are predominantly peripherally acting, i.e. have a limited ability of passing the blood-brain barrier.

### DESCRIPTION OF THE INVENTION

The present invention relates to a compound of formula I:

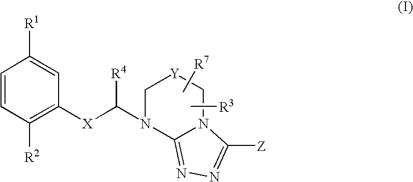

wherein

### R¹ is methyl, halogen or cyano;

### R² is hydrogen or fluoro;

### R³ is hydrogen, fluoro or C₁-C₃ alkyl;

### R⁴ is hydrogen or C₁-C₃ alkyl;

### Y is C₁-C₂ alkylene;

### X is

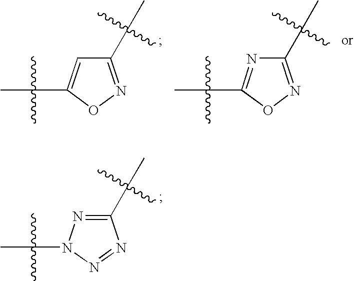

and Z is

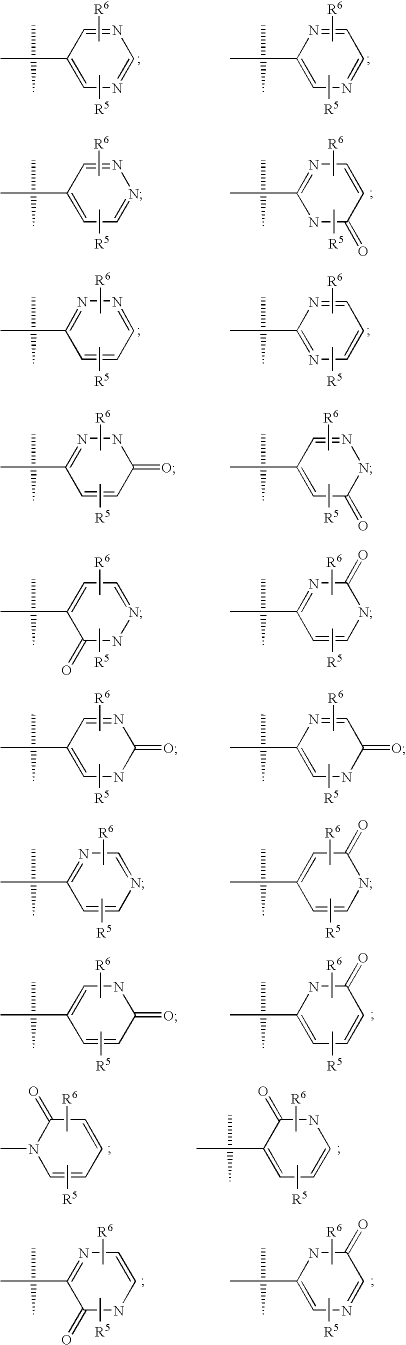

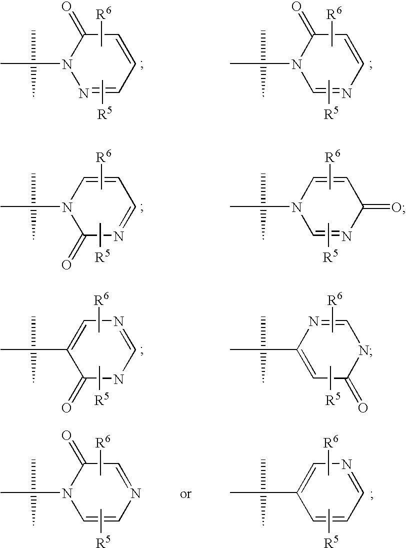

### R⁵ is hydrogen, C₁-C₃ alkyl, C₁-C₃ haloalkyl, C₁-C₃ alkoxy, C₁-C₃ haloalkoxy, C₁-C₃ amido alkyl, C₁-C₃ N′alkylamido alkyl, C₁-C₃ N′N-dialkylamido alkyl, cyano or C₁-C₃ cyanoalkyl;

### R⁶ is hydrogen, C₁-C₃ alkyl, C₁-C₃ haloalkyl, C₁-C₃ alkoxy, C₁-C₃ haloalkoxy, C₁-C₃ amido alkyl, C₁-C₃ N′-alkylamido alkyl, pyrazoyl, C₁-C₃ N′N-dialkylamido alkyl, cyano or C₁-C₃ cyanoalkyl;

### R⁷ is hydrogen, fluoro or C₁-C₃ alkyl;

as well as pharmaceutically acceptable salts, hydrates, isoforms, tautomers and/or enantiomers thereof;
with the proviso that the compound of formula I is not
3-{5-[3-(2,6-Dimethoxy-pyrimidin-4-yl)-6,7-dihydro-5H-[1,2,4]triazolo[4,3-a]pyrimidin-8-ylmethyl]-tetrazol-2-yl}-benzonitrile;
8-[2-(3-Chloro-phenyl)-2H-tetrazol-5-ylmethyl]-3-pyridin-3-yl-5,6,7,8-tetrahydro-4H-1,2,3a,8-tetraaza-azulene; or
8-{1-[2-(3-Chloro-phenyl)-2H-tetrazol-5-yl]-ethyl}-3-pyridin-3-yl-5,6,7,8-tetrahydro-[1,2,4]triazolo[4,3-a]pyrimidine.

In one embodiment R¹ is halogen or cyano.

In a further embodiment, R¹ is chloro. In a further embodiment, R¹ is fluoro. In a further embodiment, R¹ is cyano. In a further embodiment, R¹ is methyl.

In a further embodiment, R² is hydrogen.

In a further embodiment, R³ is hydrogen or fluoro.

In a further embodiment, R⁴ is hydrogen or methyl.

In a further embodiment, R⁵ is hydrogen, C₁-C₃ alkyl, C₁-C₃ haloalkyl, C₁-C₃ alkoxy, C₁-C₃ haloalkoxy, C₁-C₃ amido alkyl, C₁-C₃ N′alkylamido alkyl, C₁-C₃ N′N-dialkylamido alkyl or C₁-C₃ cyanoalkyl; and R⁶ is hydrogen, C₁-C₃ alkyl, C₁-C₃ haloalkyl, C₁-C₃ alkoxy, C₁-C₃ haloalkoxy, C₁-C₃ amido alkyl, C₁-C₃ N′-alkylamido alkyl, pyrazoyl, C₁-C₃ N′N-dialkylamido alkyl or C₁-C₃ cyanoalkyl;

In a further embodiment, R⁵ is hydrogen, C₁-C₂ alkyl or C₁-C₂ alkoxy.

In a further embodiment, R⁶ is hydrogen, C₁-C₂ alkyl or C₁-C₂ alkoxy.

In a further embodiment, R⁷ is C₁-C₂ alkyl or C₁-C₂ alkoxy.

In a further embodiment, wherein Y is methylene.

In a further embodiment, wherein Y is ethylene.

In a further embodiment, Z is

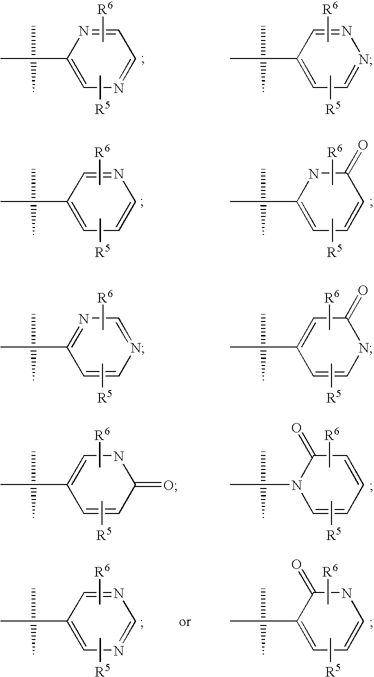

Another embodiment is a pharmaceutical composition comprising as active ingredient a therapeutically effective amount of the compound according to formula I, in association with one or more pharmaceutically acceptable diluents, excipients and/or inert carriers.

Other embodiments, as described in more detail below, relate to a compound according to formula I for use in therapy, in treatment of mGluR5 mediated disorders, in the manufacture of a medicament for the treatment of mGluR5 mediated disorders.

Still other embodiments relate to a method of treatment of mGluR5 mediated disorders, comprising administering to a mammal a therapeutically effective amount of the compound according to formula I.

In another embodiment, there is provided a method for inhibiting activation of mGluR5 receptors, comprising treating a cell containing said receptor with an effective amount of the compound according to formula I.

The compounds of the present invention are useful in therapy, in particular for the treatment of neurological, psychiatric, pain, and gastrointestinal disorders.

It will also be understood by those of skill in the art that certain compounds of the present invention may exist in solvated, for example hydrated, as well as unsolvated forms. It will further be understood that the present invention encompasses all such solvated forms of the compounds of formula I.

Within the scope of the invention are also salts of the compounds of formula I. Generally, pharmaceutically acceptable salts of compounds of the present invention are obtained using standard procedures well known in the art, for example, by reacting a sufficiently basic compound, for example an alkyl amine with a suitable acid, for example, HCl, acetic acid or a methanesulfonic acid, to afford a salt with a physiologically acceptable anion. It is also is possible to make a corresponding alkali metal (such as sodium, potassium, or lithium) or an alkaline earth metal (such as a calcium) salt by treating a compound of the present invention having a suitably acidic proton, such as a carboxylic acid or a phenol, with one equivalent of an alkali metal or alkaline earth metal hydroxide or alkoxide (such as the ethoxide or methoxide), or a suitably basic organic amine (such as choline or meglumine) in an aqueous medium, followed by conventional purification techniques. Additionally, quaternary ammonium salts can be prepared by the addition of alkylating agents, for example, to neutral amines.

In one embodiment of the present invention, the compound of formula I may be converted to a pharmaceutically acceptable salt or solvate thereof, particularly, an acid addition salt such as a hydrochloride, hydrobromide, phosphate, acetate, fumarate, maleate, tartrate, citrate, methanesulphonate or p-toluenesulphonate.

The general terms used in the definition of formula I have the following meanings:

Halogen as used herein is selected from chlorine, fluorine, bromine or iodine.

C₁-C₃ alkyl is a straight or branched alkyl group, having from 1 to 3 carbon atoms, for example methyl, ethyl, n-propyl or isopropyl.

C₁-C₃ alkoxy is an alkoxy group having 1 to 3 carbon atoms, for example methoxy, ethoxy, isopropoxy or n-propoxy.

C₁-C₃ haloalkoxy is an alkoxy group having 1 to 3 carbon atoms, for example methoxy, ethoxy or n-propoxy wherein at least one of the carbon atoms is substituted by a halogen atom.

C₁-C₃ amidoalkyl is an amido group having one a having 1 to 3 carbon atoms attached to the carbonyl of the amido function, for example NH₂CO attached via the carbon atom of the amide function to a methylene or ethylene group

C₁-C₃ N′alkylamido alkyl is an N-substituted amido group having 1 to 3 carbon atoms attached to the carbonyl of the amido function, for example RNHCO attached via the carbon atom of the amide function to a methylene or ethylene group

C₁-C₃ N′N-dialkylamido alkyl is an N,N-disubstituted amido group having 1 to 3 carbon atoms attached to the carbonyl of the amido function, for example R^{a}R^{b}NCO attached via the carbon atom of the amide function to a methylene or ethylene group

C₁-C₃ cyanoalkyl is a cyano group having 1 to 3 carbon atoms attached to the carbon of the cyano function, for example NCCH₂— or NCCH₂CH₂—.

Pyrazoyl is a monosubstituted pyrazol, attached through nitrogen.

All chemical names were generated using a software known as AutoNom accessed through ISIS draw.

In formula I above, X may be present in any of the two possible orientations.

### Pharmaceutical Composition

The compounds of the present invention may be formulated into conventional pharmaceutical compositions comprising a compound of formula I, or a pharmaceutically acceptable salt or solvate thereof, in association with a pharmaceutically acceptable carrier or excipient. The pharmaceutically acceptable carriers can be either solid or liquid. Solid form preparations include, but are not limited to, powders, tablets, dispersible granules, capsules, cachets, and suppositories.

A solid carrier can be one or more substances, which may also act as diluents, flavoring agents, solubilizers, lubricants, suspending agents, binders, or tablet disintegrating agents. A solid carrier can also be an encapsulating material.

In powders, the carrier is a finely divided solid, which is in a mixture with the finely divided compound of the invention, or the active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized moulds and allowed to cool and solidify.

Suitable carriers include, but are not limited to, magnesium carbonate, magnesium stearate, talc, lactose, sugar, pectin, dextrin, starch, tragacanth, methyl cellulose, sodium carboxymethyl cellulose, low-melting wax, cocoa butter, and the like.

The term composition is also intended to include the formulation of the active component with encapsulating material as a carrier providing a capsule in which the active component (with or without other carriers) is surrounded by a carrier which is thus in association with it. Similarly, cachets are included.

Tablets, powders, cachets, and capsules can be used as solid dosage forms suitable for oral administration.

Liquid form compositions include solutions, suspensions, and emulsions. For example, sterile water or water propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art. Exemplary compositions intended for oral use may contain one or more coloring, sweetening, flavoring and/or preservative agents.

Depending on the mode of administration, the pharmaceutical composition will include from about 0.05% w (percent by weight) to about 99% w, or from about 0.10% w to 50% w, of a compound of the invention, all percentages by weight being based on the total weight of the composition.

A therapeutically effective amount for the practice of the present invention can be determined by one of ordinary skill in the art using known criteria including the age, weight and response of the individual patient, and interpreted within the context of the disease which is being treated or which is being prevented.

### Medical Use

The compounds according to the present invention are useful in the treatment of conditions associated with excitatory activation of mGluR5 and for inhibiting neuronal damage caused by excitatory activation of mGluR5. The compounds may be used to produce an inhibitory effect of mGluR5 in mammals, including man.

The Group I mGluR receptors including mGluR5 are highly expressed in the central and peripheral nervous system and in other tissues. Thus, it is expected that the compounds of the invention are well suited for the treatment of mGluR5-mediated disorders such as acute and chronic neurological and psychiatric disorders, gastrointestinal disorders, and chronic and acute pain disorders.

The invention relates to compounds of formula I, as defined hereinbefore, for use in therapy.

The invention relates to compounds of formula I, as defined hereinbefore, for use in treatment of mGluR5-mediated disorders.

The invention relates to compounds of formula I, as defined hereinbefore, for use in treatment of Alzheimer's disease senile dementia, AIDS-induced dementia, Parkinson's disease, amylotropic lateral sclerosis, Huntington's Chorea, migraine, epilepsy, schizophrenia, depression, anxiety, acute anxiety, opthalmological disorders such as retinopathies, diabetic retinopathies, glaucoma, auditory neuropathic disorders such as tinnitus, chemotherapy induced neuropathies, post-herpetic neuralgia and trigeminal neuralgia, tolerance, dependency, Fragile X, autism, mental retardation, schizophrenia and Down's Syndrome.

The invention relates to compounds of formula I, as defined above, for use in treatment of pain related to migraine, inflammatory pain, neuropathic pain disorders such as diabetic neuropathies, arthritis and rheumatoid diseases, low back pain, post-operative pain and pain associated with various conditions including cancer, angina, renal or billiary colic, menstruation, migraine and gout.

The invention relates to compounds of formula I as defined hereinbefore, for use in treatment of stroke, head trauma, anoxic and ischemic injuries, hypoglycemia, cardiovascular diseases and epilepsy.

The present invention relates also to the use of a compound of formula I as defined hereinbefore, in the manufacture of a medicament for the treatment of mGluR Group I receptor-mediated disorders and any disorder listed above.

One embodiment of the invention relates to the use of a compound according to formula I in the treatment of gastrointestinal disorders.

Another embodiment of the invention relates to the use of a formula I compound for the manufacture of a medicament for inhibition of transient lower esophageal sphincter relaxations, for the treatment of GERD, for the prevention of gastroesophageal reflux, for the treatment regurgitation, for treatment of asthma, for treatment of laryngitis, for treatment of lung disease, for the management of failure to thrive, for the treatment of irritable bowel disease (IBS) and for the treatment of functional dyspepsia (FD).

Another embodiment of the present invention relates to the use of a compound of formula I for treatment of overactive bladder or urinary incontinence.

The wording “TLESR”, transient lower esophageal sphincter relaxations, is herein defined in accordance with Mittal, R. K., Holloway, R. H., Penagini, R., Blackshaw, L. A., Dent, J., 1995; Transient lower esophageal sphincter relaxation. Gastroenterology 109, pp. 601-610.

The wording “reflux” is herein defined as fluid from the stomach being able to pass into the esophagus, since the mechanical barrier is temporarily lost at such times.

The wording “GERD”, gastro-esophageal reflux disease, is herein defined in accordance with van Heerwarden, M. A., Smout A. J. P. M., 2000; Diagnosis of reflux disease. Baillière's Clin. Gastroenterol. 14, pp. 759-774.

The compounds of formula I above are useful for the treatment or prevention of obesity or overweight, (e.g., promotion of weight loss and maintenance of weight loss), prevention or reversal of weight gain (e.g., rebound, medication-induced or subsequent to cessation of smoking), for modulation of appetite and/or satiety, eating disorders (e.g. binge eating, anorexia, bulimia and compulsive) and cravings (for drugs, tobacco, alcohol, any appetizing macronutrients or non-essential food items).

The invention also provides a method of treatment of mGluR5-mediated disorders and any disorder listed above, in a patient suffering from, or at risk of, said condition, which comprises administering to the patient an effective amount of a compound of formula I, as hereinbefore defined.

The dose required for the therapeutic or preventive treatment of a particular disorder will necessarily be varied depending on the host treated, the route of administration and the severity of the illness being treated.

In the context of the present specification, the term “therapy” and “treatment” includes prevention or prophylaxis, unless there are specific indications to the contrary. The terms “therapeutic” and “therapeutically” should be construed accordingly.

In this specification, unless stated otherwise, the term “antagonist” and “inhibitor” shall mean a compound that by any means, partly or completely, blocks the transduction pathway leading to the production of a response by the ligand.

The term “disorder”, unless stated otherwise, means any condition and disease associated with metabotropic glutamate receptor activity.

One embodiment of the present invention is a combination of a compound of formula I and an acid secretion inhibiting agent. A “combination” according to the invention may be present as a “fix combination” or as a “kit of parts combination”. A “fix combination” is defined as a combination wherein the (i) at least one acid secretion inhibiting agent; and (ii) at least one compound of formula I are present in one unit. A “kit of parts combination” is defined as a combination wherein the (i) at least one acid secretion inhibiting agent; and (ii) at least one compound of formula I are present in more than one unit. The components of the “kit of parts combination” may be administered simultaneously, sequentially or separately. The molar ratio of the acid secretion inhibiting agent to the compound of formula I used according to the invention in within the range of from 1:100 to 100:1, such as from 1:50 to 50:1 or from 1:20 to 20:1 or from 1:10 to 10:1. The two drugs may be administered separately in the same ratio. Examples of acid secretion inhibiting agents are H2 blocking agents, such as cimetidine, ranitidine; as well as proton pump inhibitors such as pyridinylmethylsulfinyl benzimidazoles such as omeprazole, esomeprazole, lansoprazole, pantoprazole, rabeprazole or related substances such as leminoprazole.

### Non-Medical Use

In addition to their use in therapeutic medicine, the compounds of formula I, as well as salts and hydrates of such compounds, are useful as pharmacological tools in the development and standardisation of in vitro and in vivo test systems for the evaluation of the effects of inhibitors of mGluR related activity in laboratory animals such as cats, dogs, rabbits, monkeys, rats and mice, as part of the search for new therapeutic agents.

### Methods of Preparation

Another aspect of the present invention provides a process for preparing a compound of formula I or salt thereof.

Throughout the following description of such processes it is to be understood that, where appropriate, suitable protecting groups will be added to, and subsequently removed from, the various reactants and intermediates in a manner that will be readily understood by one skilled in the art of organic synthesis. Conventional procedures for using such protecting groups as well as examples of suitable protecting groups are described, for example, in “Protective Groups in Organic Synthesis”, T. W. Green, P. G. M. Wuts, Wiley-Interscience, New York, 1999. Throughout the following description of such processes it is to be understood that cross-couplings can be performed in a manner that will be readily understood by one skilled in the art of organic synthesis. Conventional procedures for cross-coupling are described, for example, in “Organometallics in Synthesis”, M. Schlosser (Ed.), John Wiley and Sons (2001).

### Abbreviations:

atm Atmosphere
aq. Aqueous

### BINAP 2,2′-bis(diphenylphosphino)-1,1′-binaphthyl

### Boc tert-butoxycarbonyl

### CDI N,N′-Carbonyldiimidazole

### DCC N,N-Dicyclohexylcarbodiimide

### DCM Dichloromethane

### DBU Diaza(1,3)bicyclo[5.4.0]undecane

### DEA N,N-Diisopropyl ethylamine

### DIBAL-H Diisobutylaluminium hydride

### DIC N,N′-Diisopropylcarbodiimide

### DMAP N,N-Dimethyl-4-aminopyridine

### DMF Dimethylformamide

### DMSO Dimethylsulfoxide

### DPPF Diphenylphosphinoferrocene

### EA Ethyl acetate

### EDCI N-[3-(dimethylamino)propyl]-N′-ethylcarbodiimide hydrochloride

### EDC 1-Ethyl-3-(3-dimethylaminopropyl)carbodiimide

### Et₂O Diethyl ether

### EtOAc Ethyl acetate

### EtOH Ethanol

### EtI Iodoethane

### Et Ethyl

### Fmoc 9-fluorenylmethyloxycarbonyl

h hour(s)

### HetAr Heteroaryl

### HOBt N-Hydroxybenzotriazole

### HBTU O-(Benzotriazol-1-yl)-N,N,N′,N′-tetramethyluronium hexafluorophosphate

### HPLC High performance liquid chromatography

### LAH Lithium aluminium hydride

### LCMS HPLC mass spec

### MCPBA m-Chlorbenzoic acid

### MeCN Acetonitrile

### MeOH Methanol

min Minutes

### MeI Iodomethane

### MeMgCl Methyl magnesium chloride

### Me Methyl

n-BuLi 1-Butyllithium

### NaOAc Sodium acetate

### NMR Nuclear magnetic resonance

### NMP N-Methyl pyrrolidinone

nBuLi 1-Butyl lithium
o.n. Over night
RT, rt, r.t. Room temperature

### TEA Triethylamine

### THF Tetrahydrofurane

nBu normal Butyl

### OMs Mesylate or methane sulfonate ester

### OTs Tosylate, toluene sulfonate or 4-methylbenzene sulfonate ester

### PCC Pyridinium chlorochromate

### PPTS Pyridinium p-toluenesulfonate

### TBAF Tetrabutylammonium fluoride

pTsOH p-Toluenesulfonic acid

### SPE Solid phase extraction (usually containing silica gel for mini-chromatography)

sat. Saturated

### General syntheses of 1,2,4-oxadiazole compounds of formula I

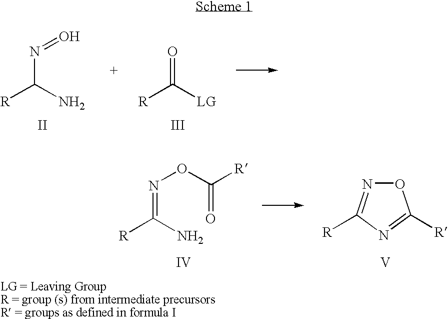

A compound of formula I, wherein X is a 1,2,4-oxadiazole (V) may be prepared through cyclization of a compound of formula IV, which in turn may be formed from a suitably activated compound of formula III with a compound of formula II.

Compounds of formula II may be prepared from a suitable nitrile, The compound of formula III may be activated in the following non-limiting ways: I) as the acid chloride formed from the acid using a suitable reagent such as oxalyl chloride or thionyl chloride; ii) as an anhydride or mixed anhydride formed from treatment with a reagent such as alkyl chloroformate; iii) using traditional methods to activate acids in amide coupling reactions such as EDCI with HOBt or uronium salts like HBTU; iv) as an alkyl ester when the hydroxyamidine is deprotonated using a strong base like sodium tert-butoxide or sodium hydride in a solvent such as ethanol or toluene at elevated temperatures (50-110° C.). This transformation of compounds II and III into compounds of type V may be performed as two consecutive steps via an isolated intermediate of type IV, as described above, or the cyclization of the intermediate formed in situ may occur spontaneously during the ester formation. The formation of ester IV may be accomplished using an appropriate aprotic solvent such as dichloromethane, tetrahydrofuran, N,N-dimethylformamide or toluene, with optionally an appropriate organic base such as triethylamine, diisopropylethylamine and the like or an inorganic base such sodium bicarbonate or potassium carbonate. The cyclization of compounds of formula IV to form an oxadiazole may be carried out on the crude ester with evaporation and replacement of the solvent with a higher boiling solvent such as DMF or with aqueous extraction to provide a semi-purified material or with material purified by standard chromatographic methods. The cyclization may be accomplished by heating conventionally or by microwave irradiation (100-180° C.), in a suitable solvent such as pyridine or N,N-dimethylformamide or using a lower temperature method employing reagents like tetrabutylammonium fluoride in tetrahydrofuran or by any other suitable known literature method.

Further examples of the above described reactions can be found in Poulain et al., Tetrahedron Lett., (2001), 42, 1495-98, Ganglott et al., Tetrahedron Lett., (2001), 42, 1441-43, and Mathvink et al., Bioorg. Med. Chem. Lett. (1999), 9, 1869-74, which are hereby included as references.

### Synthesis of Nitriles and Acids for Use in Preparation of Compounds of Formula I

Aryl nitrites are available by a variety of methods including cyanation of an aryl halide or triflate under palladium or nickel catalysis using an appropriate cyanide source such as zinc cyanide in an appropriate solvent such as N,N-dimethylformamide. The corresponding acid is available from the nitrile by hydrolysis under either acidic or basic conditions in an appropriate solvent such as aqueous alcohols. Aryl acids are also available from a variety of other sources, including iodo- or bromo-lithium exchange followed by trapping with CO₂ to give directly the acid.

Carboxylic acids may be converted to primary amides using any compatible method to activate the acid, including via the acid chloride or mixed anhydride, followed by trapping with any source of ammonia, including ammonium chloride in the presence of a suitable base, ammonium hydroxide, methanolic ammonia or ammonia in an aprotic solvent such as dioxane. This amide intermediate may be converted to the nitrile using a variety of dehydration reagents such as oxalyl chloride or thionyl chloride. This reaction sequence to convert an acid into a nitrile may also be applied to non-aromatic acids, including suitably protected amino acid derivatives. A suitable protecting group for an amine, in an amino acid or in a remote position of any other acid starting material, may be any group which removes the basicity and nucleophilicity of the amine functionality, including such carbamate protecting group as Boc.

Some acids are more easily prepared taking advantage of commercially available analogs. For example, 6-methylpyridine-4-carboxylic acid is prepared by dechlorination of 2-chloro-6-methylpyridine-4-carboxylic acid. Certain types of substituted fluoro-benzonitriles and benzoic acids are available from bromo-difluoro-benzene via displacement of one fluoro group with a suitable nucleophile such as imidazole in the presence of a base such as potassium carbonate in a compatible solvent such as N,N-dimethylformamide at elevated temperatures (80-120° C.) for extended periods of time. The bromo group may subsequently be elaborated into the acid or nitrile as above.

1,3-Disubstituted and 1,3,5-trisubstituted benzoic acids and benzonitriles may be prepared by taking advantage of readily available substituted isophthalic acid derivatives. Monohydrolysis of the diester allows selective reaction of the acid with a variety of reagents, most typically activating agents such as thionyl chloride, oxalyl chloride or isobutyl chloroformate and the like. From the activated acid, a number of products are available. In addition to the primary amide used to form the nitrite by dehydration as mentioned above, reduction to the hydroxymethyl analog may be carried out on the mixed anhydride or acid chloride using a variety of reducing agents such as sodium borohydride in a compatible solvent such as tetrahydrofuran. The hydroxymethyl derivative may be further reduced to the methyl analog using catalytic hydrogenation with an appropriate source of catalyst such as palladium on carbon in an appropriate solvent such as ethanol. The hydroxymethyl group may also be used in any reaction suitable for benzylic alcohols such as acylation, alkylation, transformation to halogen and the like. Halomethylbenzoic acids of this type may also be obtained from bromination of the methyl derivative when not commercially available. Ethers obtained by alkylation of the hydroxymethyl derivatives may also be obtained from the halomethylaryl benzoate derivatives by reaction with the appropriate alcohol using an appropriate base such as potassium carbonate or sodium hydroxide in an appropriate solvent such as tetrahydrofuran or the alcohol. When other substituents are present, these may also be employed in standard transformation reactions. Treatment of anilines with acid and sodium nitrite may yield a diazonium salt, which may be transformed into a halide such as fluoride using tetrafluoroboric acid. Phenols react in the presence of a suitable base such as potassium carbonate with alkylating agents to form aromatic ethers.

### Formation of Isoxazole Precursor of Compounds of Formula I

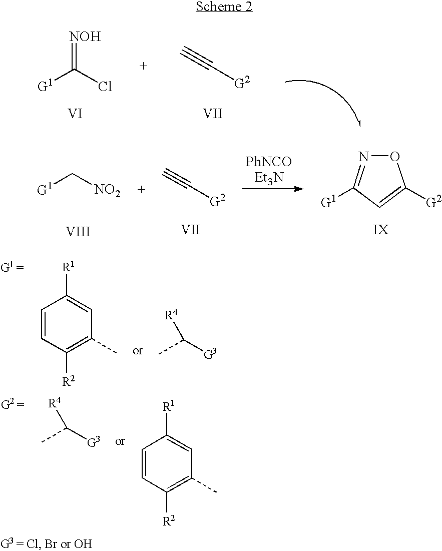

A compound of formula IX, wherein G1 and/or G2 is a moiety from an intermediate or group(s) as defined by formula I may be prepared by a 1,3-dipolar cycloaddition between compounds of formula VIII and VII under basic conditions using a suitable base such as sodium bicarbonate or triethylamine at suitable temperatures (0° C.-100° C.) in solvents such as toluene. Synthesis of compounds of type VI has previously been described in the literature, e.g. Kim, Jae Nyoung; Ryu, Eung K; J. Org. Chem. (1992), 57, 6649-50. 1,3-Dipolar cycloaddition with acetylenes of type VII can also be effected using substituted nitromethanes of type VIII via activation with an electrophilic reagent such as PhNCO in the presence of a base such as triethylamine at elevated temperatures (50-100° C.). Li, C-S.; Lacasse, E.; Tetrahedron Lett. (2002) 43; 3565-3568. Several compounds of type VII are commercially available, or may be synthesized by standard methods as known by one skilled in the art.

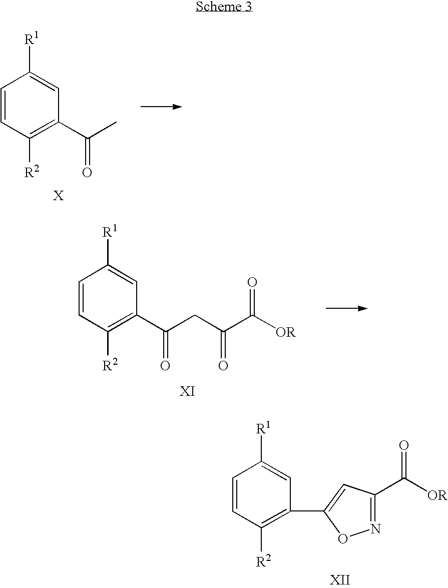

Alternatively, compounds of formula I, which are available from a Claisen condensation of a methyl ketone X and an ester using basic conditions (see Scheme 3) using such bases as sodium hydride or potassium tert-butoxide, may yield compounds of formula XI via condensation and subsequent cyclization using hydroxylamine, for example in the form of the hydrochloric acid salt, at elevated temperatures (60-120° C.) to afford intermediate XII. It is understood that for both methods, subsequent functional group transformations of intermediates such as IX and XII may be necessary. In the case of an ester group as in XII, these transformations may include, but is not limited to either of the following three procedures: a) Complete reduction using a suitable reducing agent such as LAH in solvents such as THF. b) Partial reduction using a suitable selective reducing agent such as DIBAL followed by addition of an alkylmetal reagent. c) Addition of an alkylmetal reagent such as an alkyl magnesium halide in solvents such as toluene or THF, followed by reduction with for example sodium borohydride in methanol.

### Formation of Tetrazole Precursors of Compounds of Formula I

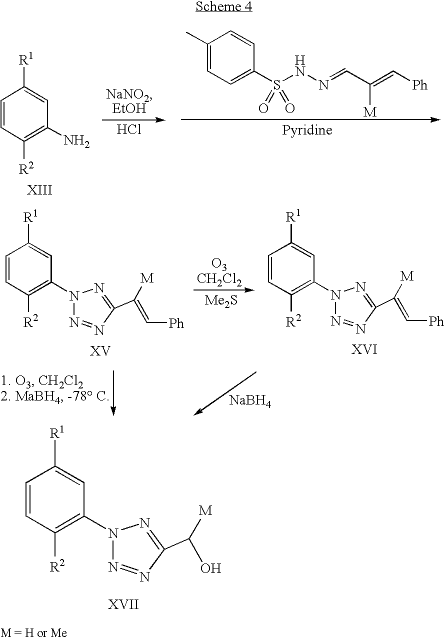

Compounds of formula I wherein X is tetrazole, as in intermediates XVI (M=H or Methyl) are prepared through condensation between arylsulphonylhydrazones XIV with diazonium salts derived from anilines XIII (Scheme 4). The tetrazole intermediate XV, obtained from the diazonium salt of XIII and the arylsulphonylhydrazones of cinnamaldehydes (M=H or Me) can be cleaved to provide an aldehyde (M=H) or ketone (M=Me) XV directly in a one-pot process using a reagent such as ozone or via the diol using a dihydroxylation reagent such as osmium tetroxide followed by subsequent cleavage using a reagent such as lead (IV) acetate. [J. Med. Chem. 2000, 43, 953-970]

The olefin can also be converted in one pot to the alcohol via ozonolysis followed by reduction with a reducing agent such as sodium borohydride. Aldehydes XV (M=H) may be reduced to primary alcohols of formula XVII (M=H) using well known reducing agents such as sodium or lithium borohydride, in a solvent such as methanol, THF or DMF at temperatures between 0-80° C. Secondary alcohols wherein M is not H may also be formed from aldehydes of formula XVI (M=H) via addition reactions of an organometallic reagent, for example Grignard reagents (e.g. MeMgX), in a solvent such as THF at temperatures between −78° C. to 80° C., and are typically performed between 0° C. and room temperature.

### Preparation of Amino[1,2,4]triazoles intermediates

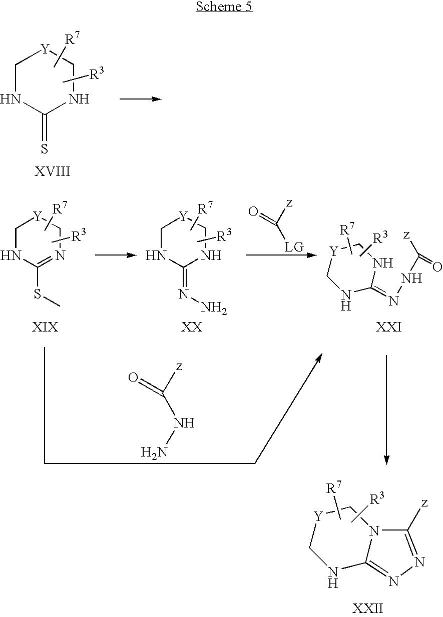

With reference to Scheme 5, amino[1,2,4]triazoles XXII are obtained by treating carbonohydrazonic diamides XX with a proper acylating agent carrying a leaving group (LG) in suitable solvent such as THF, pyridine or DMF at −20 to 100° C. The reaction initially leads to an open intermediate XXI that either forms a triazole ring spontaneously, or can be made to do so by heating at 50 to 200° C. in for example pyridine or DMF. The LG may be chloro or any other suitable LG as for example generated by in situ treatment of the corresponding acid (LG is OH) with standard activating reagents as described herein below. Carbonohydrazonic diamides XX may be generated from isothioureas XVIII, in which the S-alkyl (for example S-Me as shown in scheme 4) moiety acts as a leaving group upon treatment with hydrazine in solvents such as pyridine, methanol, ethanol, 2-propanol, THF, DMSO or the like at −20 to 180° C. The open intermediate XXI can also be directly generated by treatment of isothioureas with acylhydrazines under the same conditions as described for the reaction with hydrazine. Isothioureas are obtained by S-alkylation of the corresponding thioureas with for example MeI or EtI in acetone, EtOH, THF, DCM or the like at −100 to 100° C.

With reference to Scheme 6, alcohol intermediates may for example be converted by standard methods to the corresponding halides (e.g. LG=Cl, Br etc.) by the use of for example triphenylphosphine in combination with either iodine, N-bromosuccinimide or N-chloro-succinimide, or alternatively by treatment with phosphorous tribromide or thionyl chloride. In a similar fashion alcohols may be transformed to other LG such as mesylates or tosylates by employing the appropriate sulfonyl halide or sulfonyl anhydride in the presence of a non-nucleophilic base together with the alcohol to obtain the corresponding sulfonates. Alkyl chlorides or sulphonates can be converted to the corresponding bromides or iodides by treatment with bromide salts, for example LiBr, or iodide salts.

The subsequently described non-limiting methods of preparation of final compounds are illustrated and exemplified by drawings in which the generic groups, or other structural elements of the intermediates correspond to those of formula I. It is to be understood that an intermediate containing any other generic group or structural element than those of formula I can be used in the exemplified reactions, provided that this group or element does not hinder the reaction and that it can be chemically converted to the corresponding group or element of formula I at a later stage which is known to the one skilled in the art.

### By Connection to Nucleophilic Triazole Nitrogen

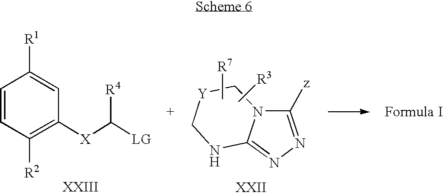

With reference to scheme 6, compounds of formula I can be prepared by bond formation through nucleophilic replacement of a leaving group (LG) in which the triazole NH moiety is acting as nucleophile. The nitrogen atom of the triazole in its anionic form, generated by treatment of the corresponding protonated neutral atom with bases in suitable solvents such as LDA or nBuLi in THF, diethyl ether or toluene, or NaH or NaOtBu in for example DMF, or K₂CO₃ in acetonitrile or ketones such as 2-butanone at a temperature from −100 to 150° C. The LG is preferably chloro, bromo, OMs and OTs. The nucleophilic reaction may also be undertaken in a stereoselective manner by employing enantiomerically pure or enriched starting materials in which the leaving group LG is attached to the stereocenter. Optionally, catalytic or stoichiometric amounts of an alkali metal iodide, such as LiI, can be present in the reaction to facilitate the same through in situ displacement of the leaving group to iodo.

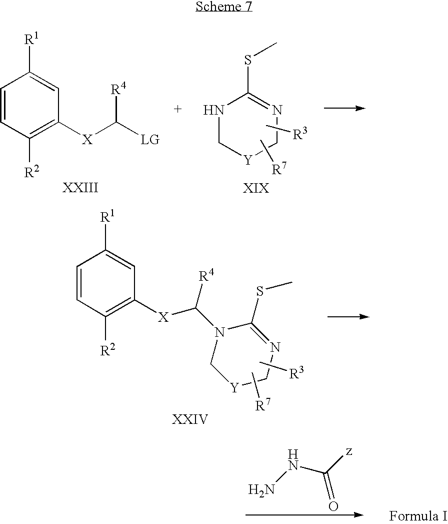

Compounds of formula I can also be prepared from intermediate XXIV by reaction with a hydrazide in a solvent like DMSO or an alcohol at a temperature from 50° C. to 150° C. according to Scheme 7. The intermediate XXIV can be formed from XXIII and XIX by treatment with a base like NaH or NaOtBu in DMF or NMP or K₂CO₃ in acetonitrile at a temperature from −100 to 150° C.

Embodiments of the present invention will now be illustrated by the following non-limiting examples.

### General Methods

All starting materials are commercially available or earlier described in the literature. The ¹H and ¹³C NMR spectra were recorded on one of a Bruker 300 at 300 MHz Bruker, DPX400 at 400 MHz or Varian +400 spectrometer at 100 MHz, using TMS or the residual solvent signal as reference. NMR measurements were made on the delta scale (δ). Mass spectra were recorded on a QTOF Global Micromass or a Waters LCMS consisting of an Alliance 2795 (LC) and a ZQ single quadropole mass spectrometer. The mass spectrometer was equipped with an electrospray ion source operated in a positive or negative ion mode. The ion spray voltage was ±3 kV and the mass spectrometer was scanned from m/z 100-700 with a scan time of 0.8 s. Column: X-Terra MS, Waters, C8, 2.1×50 mm, 3.5 μm and the column temperature was set to 40° C. A linear gradient was applied, run at 0% to 100% acetonitrile in 4 minutes, flow rate 0.3 mL/min. Mobile phase: acetonitrile/10 mM ammonium acetate in 5% acetonitrile in MilliQ Water. Preparative chromatography was run on a Gilson autopreparative HPLC with a diode array detector. Column: XTerra MS C8, 19×300 mm, 7 μm. Gradient with acetonitrile/0.1 M ammonium acetate in 5% acetonitrile in MilliQ Water, generally run from 20% to 60% acetonitrile, in 13 min. Flowrate: 20 mL/min. MS-triggered prep-LC was run on a Waters autopurification LC-MS system with a diode array detector and a ZQ mass detector. Column: XTerra MS C8, 19×100 mm, 5 μm. Gradient with acetonitrile/0.1 M ammonium acetate in 5% acetonitrile in MilliQ Water, run from 0% to 100% acetonitrile, in 10 min. Flowrate: 20 mL/min. In some cases purification by a chromatotron was performed on rotating silica gel/gypsum (Merck, 60 PF-254 with calcium sulphate) coated glass sheets, with coating layer of 2 mm using a TC Research 7924T chromatotron. Alternatively Chem Elut Extraction Column (Varian, cat #1219-8002) and Mega BE-SI (Bond Elut Silica) SPE Columns (Varian, cat #12256018; 12256026; 12256034) were used during purification of the products.

The microwave heating was performed in a Smith Synthesizer Single-mode microwave cavity producing continuous irradiation at 2450 MHz (Personal Chemistry AB, Uppsala, Sweden).

### EXAMPLES

The invention will now be illustrated by the following non-limiting examples.

### Example 1

### 2-Chloro-N-hydroxy-acetamidine

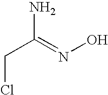

Using a modification of the procedure of Shine et al., J Heterocyclic Chem. (1989) 26:125-128, a solution of chloroacetonitrile (20 g, 265 mmol), hydroxylamine hydrochloride (18.4 g, 265 mmol) and water (66 mL) were cooled to 15° C. using a cold water bath. Sodium carbonate (14 g, 132 mmol) was added portion-wise to the reaction mixture, keeping the temperature below 30° C. The reaction mixture was stirred at 30° C. for 1 h using a warm water bath. Solid sodium chloride was added to the reaction mixture. The aqueous phase was extracted with diethyl ether (4 times 150 mL). Combined organic phase was dried (sodium sulfate), filtered and concentrated in vacuo. Crude residue was triturated with a mixture of diethyl ether in hexanes to isolate the title compound (13.5 g) as a lemon yellow solid.

¹H NMR (CDCl₃): δ (ppm) 4.71 (broad s, 2H), 4.04 (s, 2H).

### Example 2

### 3-Chloromethyl-5-m-tolyl-[1,2,4]oxadiazole

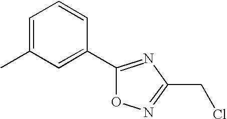

3-Methyl-benzoyl chloride (802 μL, 6.1 mmol) was added to a suspension of 2-chloro-N-hydroxy-acetamidine (440 mg, 4.1 mmol) in dichloromethane (10 mL) at room temperature. After stirring for 30 min., triethylamine (622 μL, 4.5 mmol) was added and stirred for an additional hour. The reaction mixture was diluted with dichloromethane, washed with water and brine, dried over anhydrous sodium sulfate, filtered and concentrated in vacuo. Flash column chromatography using 10-20% ethyl acetate in hexanes afforded 814 mg of the acyclic ester intermediate. DMF was added to this intermediate and then heated at 135° C. for 4 h to effect cyclization to oxadiazole. After cooling the reaction mixture washed with water (3 times) and brine, dried over anhydrous sodium sulfate, filtered, and concentrated. Purification by flash column chromatography on silica gel using 5% ethyl acetate in hexanes afforded the title compound 469 mg (54% over 2 steps) as a white solid.

¹H NMR (CDCl₃): δ (ppm) 7.99 (s, 1H), 7.97 (m, 1H), 7.43 (d, 2H), 4.68 (s, 2H), 2.45 (s, 3H).

### Example 3

### 3-(3-Chloromethyl-[1,2,4]oxadiazol-5-yl)-benzonitrile

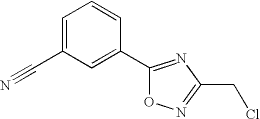

The title compound was prepared as described for Example 2 using the title compound of Example 1 (4.05 g, 37.4 mmol) and 3-cyanobenzoyl-chloride (6.2 g, 37.4 mmol) to give 3.57 g (43%).

¹H NMR (CDCl₃): δ (ppm) 8.47 (broad s, 1H), 8.41 (dd, 1H), 7.91 (dd, 1H), 7.72 (t, 1H), 4.70 (s, 2H); GC-MS (M+): 219.

### Example 4

### 3-Chloromethyl-5-(3-chloro-phenyl)-1,2,4-oxadiazole

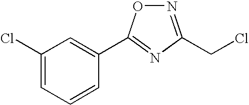

3-Chlorobenzoic acid (2.82 g, 18 mmol), EDCI (3.46 g, 18 mmol), HOBt (2.76 g, 18 mmol) and the title compound of Example 1 (1.75 g, 16.2 mmol) [Chem. Ber. 1907, 40, 1639] in DMF (40 mL). The resulting intermediate was heated at 135° C. in DMF (40 mL). Purification by SPE chromatography on silica gel using 2% acetone in hexanes yielded the title compound (1.46 g, 39% yield).

¹H NMR (300 MHz, CDCl₃): δ (ppm) 8.17 (m, 1H), 8.07 (dd, 1H), 7.60 (m, 1H), 7.55 (t, 1H), 4.69 (s, 2H).

### Example 5

### 1-[5-(3-chlorophenyl)-1,2,4-oxadiazol-3-yl]ethyl methanesulfonate

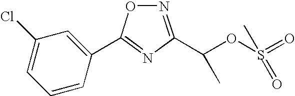

### Step A: N′,2-dihydroxypropanimidamide

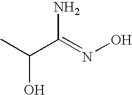

Hydroxylamine hydrochloride, 44.2 g (0.64 mol) and 25.5 g (0.64 mol) sodium hydroxide were dissolved in ethanol (500 mL) at r.t. and stirred for 3 h. After filtration, 8.11 g (0.11 mol) 2-hydroxypropanenitrile were added to the filtrate, followed by stirring for 4 h. After concentration to dryness the subtitle compound was obtained which was directly used in the next step.

¹H NMR (DMSO-d6): δ (ppm) 8.88 (s, 1H), 5.15 (s, 1H), 5.02 (s, 1H), 4.00 (q, 1H), 1.19 (d, 3H).

### Step B: 1-[5-(3-chlorophenyl)-1,2,4-oxadiazol-3-yl]ethanol

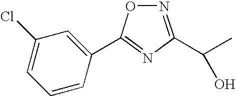

The crude material from Step A (6.45 g) was cooled on an ice-bath with 23.5 mL DEA in THF (200 mL). To this slurry 21.94 g 3-chlorobenzoyl chloride was added. The mixture was warmed to r.t. and stirred for 2 h. Addition of Et₂O (200 mL), washing with sat. aq. NH₄Cl and re-extraction of the aq. layer gave after combining and concentration of the organic layers followed by drying in vacuo 27.24 g, which was directly used in the next step. The material was dissolved in ethanol (250 mL) and refluxed for 1 h, followed by addition of 14.0 g (170 mmol) sodium acetate in water (40 mL). After refluxing over night, cooling to r.t. and addition of water (250 mL) the mixture was concentrated in vacuo to about ½ of its volume, resulting in a precipitate which was filtered off and recrystallized from EtOAc/heptane to yield 6.45 g (25%) of the subtitle compound.

¹H NMR (300 MHz, CDCl₃): δ (ppm) 8.14 (s, 1H), 8.02 (d, 1H), 7.57 (d, 1H), 7.47 (t, 1H), 5.04-5.14 (m, 1H), 2.51 (d, 1H), 1.67 (d, 3H).

### Step C: 1-[5-(3-chlorophenyl)-1,2,4-oxadiazol-3-yl]ethyl methanesulfonate

Methane sulfonyl chloride (40 μl, 0.49 mmol) was added to a mixture of TEA (95 μl, 0.67 mmol) and the subtitle compound of Step 5B (100 mg, 0.45 mmol) in DCM (5 mL). After stirring for 15 min the mixture washed with water and brine, dried and concentrated and the title compound was obtained in 135 mg yield.

¹H NMR (300 MHz, CDCl₃): δ (ppm) 8.1 (t, 1H), 8.0 (m, 1H), 7.6 (m, 1H), 7.5 (t, 1H), 5.9 (q, 1H), 3.1 (s, 3H), 1.9 (d, 3H).

### Example 6.1

### 4-(3-Chloro-phenyl)-2,4-dioxo-butyric acid ethyl ester

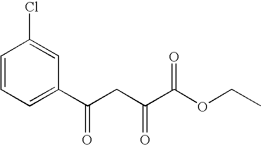

Sodium hydride (60% oil dispersion, 1.24 g, 31.1 mmol) was added in portions to a solution of 3-chloroacetophenone (4.0 g, 25.9 mmol) and diethyl oxalate (4.54 g, 31.1 mmol) in DMF (32 mL) at 0° C. The mixture stirred at room temperature for 1 hour and was then heated at 80° C. for a half an hour. After cooling, the mixture was treated with 3N HCl and then diluted with ethyl acetate. The organic layer washed with water (three times) and saturated brine, dried over anhydrous sodium sulfate, filtered and concentrated. The resulting residue was then purified by flash column chromatography on silica using 0-10% ethyl acetate in hexanes to afford of the title compound (4.43 g, 67%, yellow solid).

¹H NMR (CDCl₃): δ (ppm) 15.12 (broad s, 1H), 7.98 (s, 1H), 7.88 (d, 1H), 7.58 (d, 1H), 7.47 (t, 1H), 7.05 (s, 1H), 4.39 (m, 2H), 1.41 (m, 3H).

The example below was prepared according to the procedure for Example 6.1

| 6.2 | 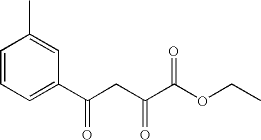 | 4-(3-Methyl-phenyl)-2,4-dioxo-butyric acid ethylester | 81%6.61 gYellowsolid |
|---|---|---|---|
| ¹H NMR | ¹H NMR (CDCl₃) δ (ppm): 15.12 (broad s, 1H), 7.81 (m, 2H), 7.43 (m, | | |
| | 2H), 7.15 (s, 1H), 3.91 (s, 3H), 2.46 (s, 3H) | | |
| 6.3 | 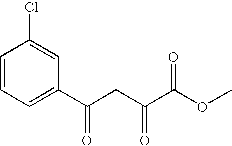 | 4-(3-Chloro-phenyl)-2,4-dioxo-butyric acid methylester | 85%33.3 gOff-whitesolid |

| | |
|---|---|
| ¹H NMR | ¹H NMR (CDCl₃) δ (ppm): 7.99 (m, 1H), 7.89 (dt, 1H), 7.60 (dt, 1H), |
| | 7.48 (t, 1H), 4.72 (broad s, 2H), 3.98 (s, 3H). |

### Example 7.1

### 5-(3-Chloro-phenyl)-isoxazole-3-carboxylic acid ethyl ester

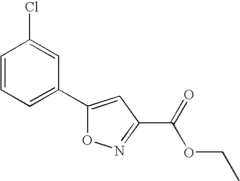

A solution of the title compound of Example 6.1 (3.0 g, 11.8 mmol) and hydroxylamine hydrochloride (2.46 g, 35.4 mmol) in methanol (60 mL) was heated at 80° C. for 4 hours. After cooling, the mixture was filtered and washed with cold methanol to afford the title compound in mixture with the methyl ester (2.0 g, 71%, white solid).

¹H NMR (300 MHz, CDCl₃): δ (ppm) 7.82 (s, 1H), 7.72 (m, 1H), 7.47 (m, 2H), 4.03 (s, 3H).

The example below was prepared according to the procedure for Example 7.1

| 7.2 | 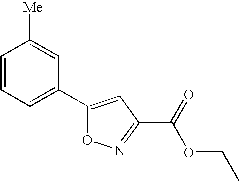 | 5-(3-Methyl-phenyl)-isoxazole-3-carboxylic acidethyl ester | 100%6.51 gWhite solid |
|---|---|---|---|
| ¹H NMR | (300 MHz, CDCl₃): δ (ppm) 7.64 (s, 1H), 7.60 (d, 1H), 7.38 (t, 1H), 7.29 | | |
| | (d, 1H), 6.93 (s, 1H), 4.01 (s, 3H), 2.43 (s, 3H) | | |
| 7.3 | 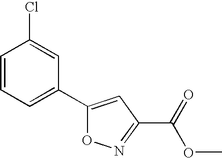 | 5-(3-Chloro-phenyl)-isoxazole-3-carboxylic acidmethyl ester | 61.3%20.0 gOff-whitesolid |

### Example 8.1

### [5-(3-Chloro-phenyl)-isoxazol-3-yl]-methanol

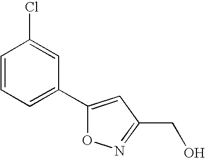

Lithium aluminum hydride (320 mg, 8.4 mmol) was slowly added to a solution of the mixture obtained in Example 7.1 (2.0 g, 8.4 mmol) in THF (100 mL) at room temperature. After 1 hour, the reaction mixture was quenched with water and then extracted with ethyl acetate. The organic layer washed with water and saturated brine, dried over anhydrous sodium sulfate, filtered, and concentrated. The resulting residue was then purified by flash column chromatography using 15-40% ethyl acetate in hexane to afford the title compound (1.32 g, 75%, yellow solid).

¹H NMR (300 MHz, CDCl₃): δ (ppm) 7.78 (s, 1H), 7.68 (m, 1H), 7.43 (m, 2H), 6.63 (s, 1H), 4.84 (d, 2H), 2.23 (t, 1H).

### Example 8.2

### [5-(3-Methyl-phenyl)-isoxazol-3-yl]-methanol

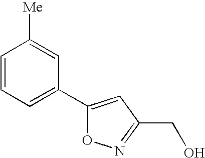

In a similar manner using DIBAL-H as the reducing agent and performing the reaction at −78° C. to 0° C., the title compound was obtained as a white solid (952 mg, 17% yield).

¹H NMR (300 MHz, CDCl₃): δ 7.62 (s, 1H), 7.60 (d, 1H), 7.37 (t, 1H), 7.26 (d, 1H), 6.59 (s, 1H), 4.84 (s, 2H)), 2.44 (s, 3H).

The example below was prepared according to the procedure for Example 8.2:

| | | | |
|---|---|---|---|
| 8.3 | 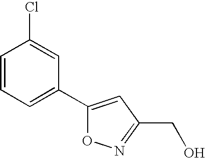 | [5-(3-Chloro-phenyl)-isoxazol-3-yl]-methanol | 100%15.8 g |

| | |
|---|---|
| ¹H NMR | (300 MHz, CDCl₃): δ (ppm) 7.78 (t, 1H), 7.66 (m, 1H), 7.43 (m, 2H), |
| | 6.63 (s, 1H), 4.84 (s, 2H), 2.11 (broad s, 1H). |

### Example 9.1

### Methanesulfonic acid 5-(3-chloro-phenyl)-isoxazol-3-ylmethyl ester

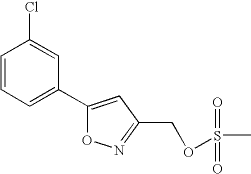

Triethyl amine (965 mg, 9.5 mmol) and methanesulfonyl chloride (820 mg, 7.2 mmol) were added to a solution of the title compound of Example 8.1 (1.0 g, 4.8 mmol) in DCM (50 mL) at 0° C. After 1 hour, the reaction mixture was quenched with cold saturated sodium bicarbonate and then the organic layer washed with saturated brine, dried over anhydrous sodium sulfate, filtered, and concentrated to afford the title compound as a light brown solid (1.4 g, 100%).

¹H NMR (300 MHz, CDCl₃): δ (ppm) 7.80 (s, 1H), 7.70 (m, 1H), 7.45 (m, 2H), 6.73 (s, 1H), 5.37 (s, 2H), 3.16 (s, 3H).

The example below was prepared according to the procedure for Example 9.1

| | | | |
|---|---|---|---|
| 9.2 | 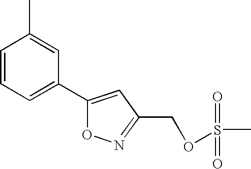 | Methanesulfonic acid 5-(3-Methyl-phenyl)-isoxazol-3-ylmethyl ester | 90%608 mgBrown oil |

| | |
|---|---|
| ¹H NMR | (300 MHz, CDCl₃): δ (ppm) 7.63 (s, 1H), 7.60 (d, 1H), 7.37 (t, 1H), 7.26 |
| | (d, 1H), 6.68 (s, 1H), 5.37 (s, 2H), 3.12 (s, 3H), 2.44 (s, 3H) |

### Example 10

### 1-[5-(3-Chloro-phenyl)-isoxazol-3-yl]-ethanone

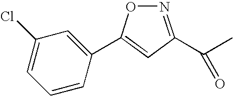

In a screw cap vial equipped with stir bar added methyl magnesium iodide (3 M in diethyl ether) (0.79 mL, 2.38 mmol), toluene (1 mL), tetrahydrofuran (0.39 mL, 4.77 mmol) and triethylamine (1 mL, 7.15 mmol). Cooled the solution down to 0° C. and to it added solution of the title compound of Example 7.1 (300 mg, 1.19 mmol) in toluene (5 mL). The resulting mixture was stirred at 0° C. for 5 h. The reaction mixture was quenched with 1 M hydrochloric acid (aqueous, 6.5 mL, 6.5 mmol), diluted with toluene (35 mL), sequentially washed with water (50 mL), saturated sodium bicarbonate (aqueous, 30 mL), water (50 mL) and brine (30 mL). The organic phase was concentrated, in vacuo. The isolated residue was dissolved in methanol (8 mL) and 20% potassium hydroxide (aqueous, 1 mL). The mixture was stirred at 45° C. for 30 minutes. At this point the mixture was concentrated, in-vacuo. The isolated residue was dissolved in toluene (60 mL), sequentially washed with water (50 mL), saturated sodium bicarbonate (aqueous, 50 mL) and water (50 mL). The organic phase was concentrated, in-vacuo. The crude residue was purified on silica gel using 2% ethyl acetate in hexanes to isolate the title compound as a white solid (156 mg, 60%).

¹H NMR (300 MHz, CDCl₃): δ (ppm) 7.77 (m, 1H), 7.66 (m, 1H), 7.42 (m, 2H), 6.90 (s, 1H), 2.69 (s, 3H).

### Example 11

### Methanesulfonic acid 1-[5-(3-chloro-phenyl)-isoxazol-3-yl]-ethyl ester

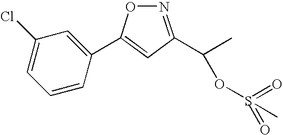

### Step A: 1-[5-(3-Chloro-phenyl)-isoxazol-3-yl]-ethanol

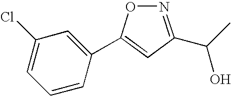

In a screw cap vial equipped with stir bar added the title compound of Example 10 (100 mg, 0.45 mmol), sodium borohydride (34 mg, 0.90 mmol) and methanol (3 mL). The resulting mixture was stirred at room temperature for 3 h. The reaction was quenched with water (30 mL) and brine (30 mL), extracted with dichloromethane (3 times 30 mL). The combined organic phase was dried (sodium sulfate), filtered and concentrated, in vacuo to isolate the subtitle compound as a white solid (110 mg).

¹H NMR (300 MHz, CDCl₃): δ (ppm) 7.69 (m, 1H), 7.59 (m, 1H), 7.37 (m, 2H), 6.59 (s, 1H), 5.07 (q, 1H), 3.45 (broad s, 1H), 1.58 (d, 3H).

### Step B

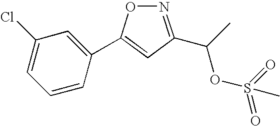

In a screw cap vial equipped with stir bar added the subtitle compound of Step 12A (110 mg, 0.49 mmol), dichloromethane (3 mL) and triethylamine (0.34 mL, 2.46 mmol). The mixture was cooled to 0° C. and to it added methane sulfonyl chloride (0.08 mL, 0.98 mmol). The reaction mixture was stirred at room temperature for 30 minutes. The reaction was quenched with saturated sodium bicarbonate (aqueous, 40 mL) and extracted with dichloromethane (3 times 30 mL). The combined organic phase washed with brine (40 mL), dried (sodium sulfate), filtered and concentrated, in vacuo to isolate the title compound as a brown oil.

¹H NMR 300 MHz, solvent): δ (ppm) 7.76 (d, 1H), 7.66 (m, 1H), 7.42 (m, 2H), 6.69 (s, 1H), 5.90 (q, 1H), 3.05 (s, 3H), 1.82 (d, 3H).

### Example 12

### 3-(3-Hydroxymethyl-isoxazol-5-yl)-benzonitrile

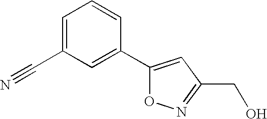

### Step A: Methyl 5-(3-iodophenyl)isoxazole-3-carboxylate

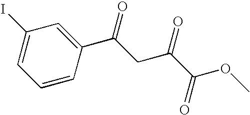

Sodium hydride (60% oil dispersion, 4.9 g, 123 mmol) was added in portions to a solution of 3-iodoacetophenone (25.18 g, 102.3 mmol) and dimethyl oxalate (14.5 g, 123 mmol) in DMF (125 mL) at 0° C. The mixture was stirred at room temperature for 1 hour and was then heated at 115° C. for 1 h. After cooling, the mixture was treated with 3 M HCl and then diluted with ethyl acetate. The organic layer washed three times with water and saturated brine, dried over anhydrous sodium sulfate, filtered and concentrated. Purification by chromatography (silica, 0-10% ethyl acetate in hexanes) afforded the title compound as a yellow solid (24.2 g, 71.3%).

¹H NMR 300 MHz, solvent): δ (ppm) 15.01 (broad s, 1H), 8.34 (d, 1H), 7.95 (m, 2H), 7.28 (s, 1H), 7.25 (m, 1H), 3.98 (s, 3H).

### Step B: 5-(3-Iodo-phenyl)-isoxazole-3-carboxylic acid methyl ester

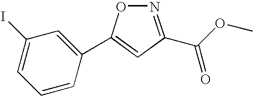

A solution of the subtitle compound of step 12A (33.9 g, 102 mmol) and hydroxylamine hydrochloride (21.3 g, 306 mmol) in methanol (450 mL) was heated at reflux for 4 hours. After cooling, the mixture was filtered and washed with cold methanol to afford the subtitle compound (24.1 g, 72%, brown solid).

¹H NMR (300 MHz, CDCl₃): δ (ppm) 8.18 (m, 1H), 7.82 (t, 2H), 7.26 (t, 1H), 6.97 (s, 1H), 4.03 (s, 3H).

### Step C: Methyl 5-(3-cyanophenyl)isoxazole-3-carboxylate

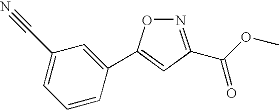

The product from Step 12B, zinc cyanide (1.0 g, 3.04 mmol), tetrakis(triphenyl-phosphine)palladium(0) (351 mg, 0.30 mmol) in DMF (10 mL) was stirred at 80° C. for 10 min. The mixture was diluted with ethyl acetate and filtered through celite, washed three times with water and saturated brine, dried over anhydrous sodium sulfate, filtered and concentrated. Purification by chromatography (silica, 5-70% ethyl acetate in hexanes) afforded the subtitle compound as a yellow solid (660 mg, 91%).

¹H NMR (300 MHz, CDCl₃): δ (ppm) 8.12 (m, 1H), 8.07 (dd, 1H), 7.81 (dd, 1H), 7.67 (dd, 1H), 7.06(s, 1H), 4.05 (s, 3H).

### Step D: [5-(3-cyanophenyl)isoxazole-3-carboxylic acid

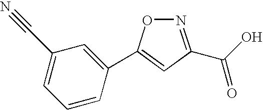

To the product from Step 12C (660 mg, 2.89 mmol) in THF (10 ml), was added LiOH (6.9 ml of a 0.5 M solution) and the mixture was stirred at 70° C. for 30 min. The mixture was cooled, diluted with water and acidified with 1N HCl to pH 2 and filtered to give 597 mg of the product as a white solid (96% yield).

¹H NMR (300 MHz, DMSO-d6): δ (ppm) 14.10 (broad s, 1H), 8.48 (s, 1H), 8.27 (d, 1H), 8.01(d, 1H), 7.78 (dd, 1H), 7.60(s, 1H).

### Step E: 3-(3-Hydroxymethyl-isoxazol-5-yl)-benzonitrile

To a suspension of product from Step 12D (497 mg, 2.3 mmol) in THF (10 mls) at 0° C. was added Et₃N (323 ul, 2.3 mmol), ethylchloroformate (222 ul, 2.3 mmol) and the reaction was stirred at 0° C. for 1 h. The mixture was filtered and NaBH₄ (219 mg, 5.8 mmol) in H₂O (5 ml) was added dropwise to the filtrate at 0° C. After the addition was complete, the reaction was stirred at 0° C. for 1.5 h and 1N HCl was added. The mixture was then diluted with ether, the organic layer washed three times with water and saturated brine, dried over anhydrous sodium sulfate, filtered and concentrated. Purification by chromatography (silica, 0-10% ethyl acetate in hexanes) afforded the title compound as a white solid (420 mg, 76%).

¹H NMR (300 MHz, CDCl₃): δ (ppm) 8.08 (d, 1H), 8.05 (dd, 1H), 7.75(dd, 1H), 7.41 (dd, 1H), 6.72(s, 1H), 4.86(d, 2H), 2.10(t, 1H).

### Example 13

### Methanesulfonic acid 5-(3-cyano-phenyl)-isoxazol-3-ylmethyl ester

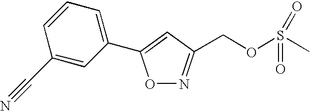

Methanesulfonyl chloride (111 ul, 1.43 mmol) and triethylamine (265 ul, 1.9 mmol) were added to a solution of 3-[3-(1-hydroxyethyl)isoxazol-5-yl]benzonitrile (200 mg, 0.95 mmol) in dichloromethane (10 mL) at 0° C. The reaction mixture was stirred at 0° C. for 30 minutes, then washed with cold saturated sodium bicarbonate. The organic layer washed with brine, dried with sodium sulfate and concentrated in vacuo to give the title compound which was used without further purification (237 mg of an off-white solid, 90%).

¹H NMR (300 MHz, CDCl₃): δ (ppm) 8.10 (d, 1H), 8.04 (dd, 1H), 7.77 (dd, 1H), 7.65 (t, 1H), 6.81 (s, 1H), 5.39 (s, 2H), 3.14 (s, 3H),

### Example 14.1

### Cinnamaldehyde tosyl hydrazone

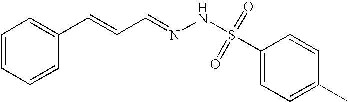

Cinnamaldehyde (8.80 g, 66.6 mmol) was added to p-toluene sulfonamide (12.44 g, 66.79 mmol) in ethanol (70 mL). The reaction immediately turned solid and ethanol (20 mL) was again added. The reaction was allowed to stir at room temperature for one hour and was then filtered. The solid washed with methanol and dried by reduced pressure to yield the title compound as a white solid (17.5 g, 87%).

¹H NMR (300 MHz, CDCl₃): δ (ppm) 8.23 (s, 1H), 7.88 (d, 2H), 7.60 (d, 1H), 7.34 (m, 6H), 6.83 (m, 2H), 2.43 (s, 3H).

### Example 14.2

### 2-Methyl Cinnamaldehyde tosyl hydrazone

2-Methyl-3-phenylacrylaldehyde (15.0 g, 102.6 mmol) was added to p-toluene sulfonamide (19.2 g, 102.9 mmol) in ethanol (70 mL). The reaction immediately turned solid and ethanol (20 mL) was again added. The reaction was allowed to stir at room temperature for 8 h and was then filtered. The solid washed with methanol and dried by reduced pressure to yield the title compound as a white solid (30.94 g, 96%).

¹H NMR (300 MHz, CD3OD): δ (ppm) 7.80 (d, 2H), 7.60 (s, 1H), 7.35 (m, 6H), 7.26 (m, 1H), 6.67 (s, 1H), 2.42 (s, 3H), 2.01 (s, 3H),

### Example 15

### 3-[5-((E)-Styryl)-tetrazol-2-yl]-benzonitrile

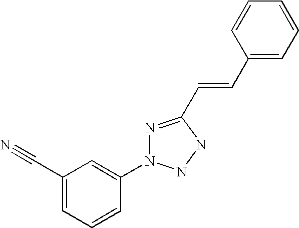

An aqueous (15 mL) solution of sodium nitrite (1.58, 22.8 mmol) was added to a solution of 3-aminobenzonitrile in water (15 mL), concentrated hydrochloric acid (10 mL) and ethanol (20 mL) via dropping funnel. The reaction was allowed to stir at 0° C. for ten minutes. This solution was poured into a dropping funnel and ice was added. This was added dropwise to a solution of cinnamaldehyde tosyl hydrazone (6.73 g, 22.4 mmol) in pyridine (60 mL). The mixture was allowed to stir overnight. An aqueous workup was done extracting with dichloromethane three times. The combined layers were washed with brine, dried over sodium sulfate, filtered and concentrated. The crude product was partially purified by column chromatography (20% EtOAc/hexanes to give 6.12 g (14% yield) of the title compound as a light purple solid that was used directly in the next step.

### Example 16.1

### 3-(3-chloro-phenyl)-5-styryl-2H-tetrazole

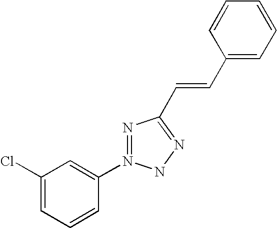

An aqueous (5 mL) solution of sodium nitrite (540.9 mg, 7.839 mmol) was added to a solution of 3-chloroaniline in water (7 mL), concentrated hydrochloric acid (3 mL) and ethanol (7 mL) via dropping funnel. The reaction was allowed to stir at 0° C. for ten minutes. This solution was poured into a dropping funnel and ice was added. This was added dropwise to a solution of cinnamaldehyde tosyl hydrazone (2.3 g, 7.7 mmol) in pyridine (20 mL). This was allowed to stir overnight. An aqueous workup was done extracting with DCM three times. The combined layers were washed with brine, dried over sodium sulfate, filtered and concentrated. The crude product was purified by column chromatography (20% EtOAc/hexanes) to yield the title compound as a light purple solid (433 mg, 19%).

¹H NMR (300 MHz, CDCl₃): [symbol] (ppm) 8.21 (m, 1H), 8.09 (dt, 1H), 7.89 (d, 1H), 7.61 (m, 2H), 7.49 (m, 5H), 7.24 (d, 1H).

### Example 16.2

### 2-(3-chlorophenyl)-5-[(E)-1-methyl-2-phenylvinyl]-2H-tetrazole

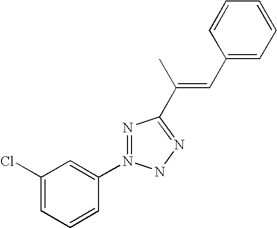

An aqueous (5 mL) solution of sodium nitrite (654 mg, 9.5 mmol) was added to a solution of 3-chloroaniline (0.92 ml, 8.7 mmol) in water (10 mL), concentrated hydrochloric acid (11.9 mL) and ethanol (7 mL) via dropping funnel. The reaction was allowed to stir at 0° C. for ten minutes. This solution was poured into a dropping funnel and ice was added. This was added dropwise to a solution of 2-methylcinnamaldehyde tosyl hydrazone (2.5 g, 7.9 mmol) in pyridine (10 mL). This was allowed to stir at 0° C. for 1.5 h. The mixture was extracted with dichloromethane three times. The combined layers were washed with brine, dried over sodium sulfate, filtered and concentrated. The crude product was purified by column chromatography (20% EtOAc/hexanes) to yield the title compound as a red solid (736 mg, 28%).

¹H NMR (CDCl₃) δ (ppm) 8.23 (s, 1H), 8.11 (dd, 1H), 7.94 (s, 1H), 7.55-7.30 (m, 7H), 2.50 (d, 3H).

### Example 17

### 5-Styryl-2-m-tolyl-2H-tetrazole

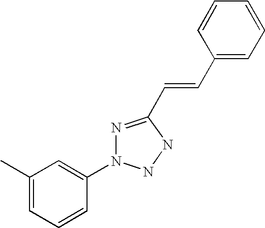

The title compound (320 mg, 30%, dark yellow solid) was obtained by adding the diazonium salt prepared from m-tolylamine (0.44 mL, 4.1 mmol) with aqueous sodium nitrite (286 mg, 4.1 mmol in 3 mL water), hydrochloric acid (5.5 mL, 17.8 mmol) in ethanol (4 mL), to a solution of cinnamaldehyde tosyl hydrazone (1.21 g, 4.1 mmol) in pyridine (30 mL). The crude product was purified by column chromatography (3-6% EtOAc/hexanes).

¹H NMR (300 MHz, CDCl₃): δ (ppm) 8.00 (s, 1H), 7.98 (d, 1H), 7.88(d, 1H), 7.63 (m, 2H), 7.38-7.47 (m, 4H), 7.33 (d, 1H), 7.26 (d, 1H), 2.55 (s, 3H).

### Example 18

### General Procedure for Ozonolysis of the Phenyl Tetrazole Intermediates Followed by Aldehyde/Ketone Reduction with Sodium Borohydride

The phenyl tetrazoles were dissolved in dichloromethane and cooled to −78° C. Ozone was bubbled through the solution for a period of 10-30 minutes. The progress of the reaction was checked using a 10% EtOAc:Hexane TLC solvent system. Once the reaction appeared complete, sodium borohydride (70 mg/mmol tetrazole) and MeOH (˜5 mL/mmol) were added to the solution. The solution was allowed to equilibrate back to room temperature and left overnight. Water (5 mL) and saturated ammonium chloride (5 mL) were added to the solution. The mixture was concentrated under low pressure and an aqueous workup was performed using DCM, water and brine. Anhydrous sodium sulfate was used to dry the solution. A standard flash column was run using a 10%-35% EtOAc:hexanes solvent system. The samples were subjected to NMR analysis. The following table represents all the reactions performed.

The examples below were prepared according to the generic procedure for Example 18.

### Example 19

### 1-[2-(3-Chloro-phenyl)-2H-tetrazol-5-yl]-ethanone

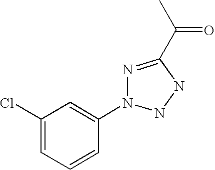

| Phenyl | | | |
|---|---|---|---|
| Tetrazole | Product | Name | Yield |
| 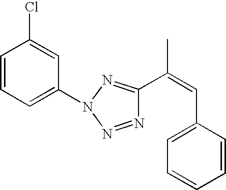 | 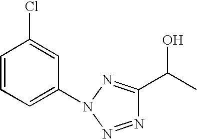 18.1 | 1-[2-(3-Chloro-phenyl)-2H-tetrazol-5-yl]-ethanol | 60%1.01 gOrange Powder |

| | |
|---|---|
| ¹H-NMR | (300 MHz, CDCl₃): δ (ppm) 8.18 (s, 1H), 8.06 (d, 1H), 7.51 (broad s, 2H), 5.32 (broad s, 1H), 2.70 |
| | (broad s, 1H), 1.78 (d, 3H) |

| | | | |
|---|---|---|---|
| 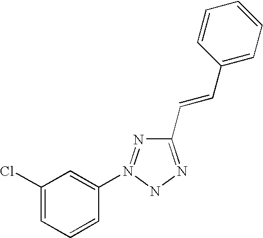 | 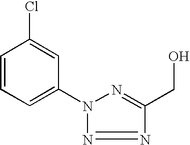 18.2 | 2-(3-Chloro-phenyl)-2H-tetrazol-5-yl]-methanol | 31%460 mgOrange Solid |

| | |
|---|---|
| ¹H-NMR | (300 MHz, CDCl₃): δ (ppm) 8.19 (s, 1H), 8.06 (m, 1H), 7.52 (m, 2H), 5.08 (d, 2H), 2.37 (t, 1H) |

The title compound of Example 16.3 (1.50 g, 5.06 mmol) was dissolved in dichloromethane (79 mL) and ozone was bubbled through the solution for a period of 15 minutes. The solution turned from orange to a darker orange colour. The reaction completeness was checked using a 10% EtOAc:hexanes TLC solvent system. Oxygen was bubbled through the solution for an additional 5 minutes to remove any excess ozone remaining. Dimethyl sulfide (5 mL) was added to the solution and the mixture was allowed to equilibrate to room temperature. The solvent was removed under vacuum and an oily brown substance remained. A 3 cm flash column was prepared containing ˜15 cm silica and ˜3 cm sand. The column was run using a 5% EtOAc:hexanes solvent system. The eluted fractions containing the product where collected and concentrated under low pressure. The product was subject to nuclear magnetic analysis. Flash column chromatography (silica, 5% EtOAc: hexanes) yielded 893 mg (79.4% yield) of the title compound.

¹H NMR (300 MHz, CDCl₃): δ (ppm) 8.22 (s, 1H), 8.11 (m, 1H), 7.54 (d, 1H), 2.85 (s, 3H).

### Example 20

### 1-[2-(3-Chloro-phenyl)-2H-tetrazol-5-yl]-2-phenyl-ethane-1,2-diol

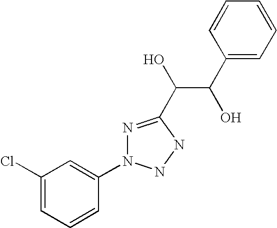

The title compound of Example 16 (127.0 mg, 0.446 mmol) was weighed into a vial and citric acid (171 mg, 0.892 mmol) was added followed by a 1:1 mixture of t-butanol and water (3 mL). Potassium osmate oxide hydrate (0.3 mg) was added followed by 4-methyl morpholine N-oxide (in 1.5 mL of water) and the reaction was allowed to stir overnight. The reaction was filtered and washed with water and 1 M hydrochloric acid to yield the title compound as a beige solid (95.4 mg, 68%).

¹H NMR (300 MHz, CD₃OD): [symbol] (ppm) 8.09 (s, 1H), 8.012 (dt, 1H), 7.58 (m, 2H), 7.25 (m, 5H), 5.15 (s, 2H).

### Example 21

### 1-Phenyl-2-(2-m-tolyl-2H-tetrazol-5-yl)-ethane-1,2-diol

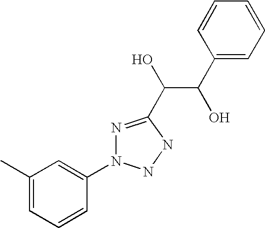

The title compound (2.26 g, used crude, yield determined after next step) was obtained from the title compound of Example 17 (1.44 g, 5.5 mmol) using citric acid (2.1 g, 10.9 mmol), potassium osmate oxide hydrate (small scoop), 4-methyl morpholine N-oxide (710 mg, 6.1 mmol) in 1:1 mixture of t-butanol and water (52 mL). The crude product from extraction was not further purified but used directly in the next step.

### Example 22

### 2-(3-Chloro-phenyl)-2H-tetrazole-5-carbaldehyde

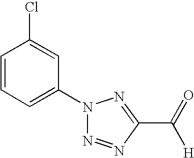

The crude product of the title compound from Example 21 (50.0 mg, 0.158 mmol) was weighed into a vial and toluene (3 mL) was added. Potassium carbonate (47.0 mg, 0.340 mmol) and lead (IV) acetate (70.0 mg, 0.158 mmol) were added with stirring. The reaction was allowed to stir for 2.5 hours. The reaction was filtered and ethyl acetate was added to the filtrate and an aqueous workup was done. The organic layer washed with brine, dried over sodium sulfate, filtered and concentrated. The crude product was purified by column chromatography (40% EtOAc/hexanes) to yield the pure product as a white solid (22.3 mg, 68%).

¹H NMR (300 MHz, CDCl₃): δ (ppm) 10.34 (s, 1H), 8.27 (s, 1H), 8.14 (m, 1H), 7.58 (d, 2H).

### Example 23

### 3-(5-Formyl-tetrazol-2-yl)-benzonitrile

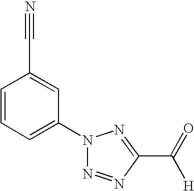

The title compound of Example 15 (400 mg, 1.46 mmol) was dissolved in dichloromethane (20 mL) and ozone was bubbled through the solution for a period of 15 minutes. The solution turned from red to a yellow colour. The reaction completeness was then checked using a 20% EtOAc:hexanes TLC solvent system. Dimethyl sulfide (1.5 mL) was then added to the solution and the mixture was allowed to equilibrate to room temperature over night. The solvent was then removed under vacuum. Flash column chromatography (silica, 20-30% EtOAc:hexanes) yielded 270 mg (91.7% yield) of product.

¹H NMR (300 MHz, CDCl₃): δ (ppm) 10.36 (s, 1H), 8.57 (s, 1H), 8.54 (d, 1H).

### Example 24

### 2-m-Tolyl-2H-tetrazole-5-carbaldehyde

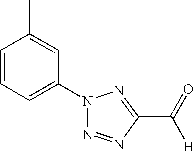

The title compound (870 mg, 84% over 2 steps) was obtained from the crude product of the title compound of Example 23 (crude from 5.5 mmol reaction above) using potassium carbonate (2.02 g, 14.6 mmol) and lead (IV) acetate (2.52 g, 5.7 mmol) in toluene (35 mL) and dichloromethane (20 mL). The crude product was purified by column chromatography (10% EtOAc/hexanes).

¹H NMR (300 MHz, CDCl₃): δ (ppm) 10.34 (s, 1H), 8.06 (s, 1H), 8.03 (d, 1H), 7.50 (t, 1H), 7.40 (d, 1H), 2.50 (s, 3H).

### Example 25

### 3-(5-Hydroxymethyl-tetrazol-2-yl)-benzonitrile

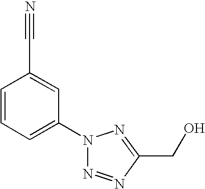

Dimethyl formamide (7 mL) was added to the title compound of Example 24 (237 mg, 1.19 mmol) and the mixture was cooled to 0° C. Et₂O (5 mL) and sodium borohydride (952 mg, 23.8 mmol) where then added to the reaction and the reaction was allowed to proceed for 15 minutes. After this period of time, the reaction was transferred to a separatory funnel and 3 M HCl (10 mL) was added drop wise to the reaction. An aqueous workup was then performed using dichloromethane, water and brine. The organic layer was dried over anhydrous sodium sulfate, filtered, and concentrated. Flash column chromatography (silica, 35% EtOAc: hexanes) gave the title compound as a white solid (201 mg, 85%)

¹H NMR (300 MHz, CDCl₃): δ (ppm) 8.47 (s, 1H), 8.45 (d, 1H), 7.81 (d, 1H).

### Example 26.1

### 1-[2-(3-chloro-phenyl)-2H-tetrazol-5-yl-ethanol

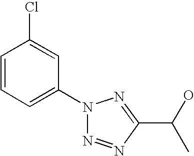

The title compound of Example 22 (75.6 mg, 0.362 mmol) was dissolved in THF (2 mL) under Argon and the flask was immersed in ice. Methyl magnesium bromide (1 M solution/butyl ether 0.51 mL, 0.507 mmol) was added dropwise while the reaction was cooled in ice. After fifteen minutes at 0° C., the ice bath was removed and the reaction was allowed to stir at room temperature for two hours. Hydrochloric acid (1 M) was added to quench the reaction and an aqueous workup was done extracting with ethyl acetate three times. The combined organic layers were washed with brine, dried over sodium sulfate, filtered and concentrated. The crude product was purified by column chromatography (3% MeOH/DCM) to yield the title compound as a clear oil (62.4 mg, 77%).

¹H NMR (300 MHz, CDCl₃): δ (ppm) 8.18 (s, 1H), 8.06 (m, 1H), 7.50 (m, 2H), 5.32 (m, 1H), 2.69 (d, 1H), 1.76 (d, 3H).

The example below was prepared according to the procedure for Example 26

| | | | |
|---|---|---|---|
| 26.2 | 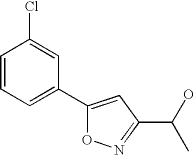 | 1-[5-(3-Chloro-phenyl)-isoxazol-3-yl]-ethanol | 80%8.84 g |

### Example 27

### (2-m-Tolyl-2H-tetrazol-5-yl)-methanol

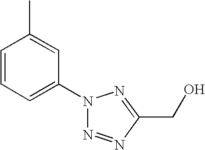

The title compound (221 mg, 96%, beige solid) was obtained from 2-m-tolyl-2H-tetrazole-5-carbaldehyde (229 mg, 1.22 mmol) using lithium borohydride (3.5 mL, 7 mmol) in THF (10 mL). The crude product was purified by column chromatography (20-30% EtOAc/hexanes).

¹H NMR (300 MHz, CDCl₃): δ (ppm) 7.97 (s, 1H), 7.94 (d, 1H), 7.46 (t, 1H), 7.33 (d, 1H), 5.08 (d, 2H), 2.50 (s, 3H), 2.40 (t, 1H).

### Example 28

### General Procedure For Tetrazole Mesylate Formation

1-[2-(3-Substituted-phenyl)-2H-tetrazol-5-yl]-(eth/meth)anol was dissolved in dichloromethane (10 mL/mmol) and cooled to 0° C. Triethylamine (2 equivalents) and mesyl chloride (1.5 equivalents) were added to the reaction and the mixture was stirred for 1 hour. Cold sodium bicarbonate was added to the solution and an aqueous workup was performed using dichloromethane and Brine. The organic layer was then dried over anhydrous sodium sulfate, filtered, and concentrated. The following table depicts the mesylations, which were performed.

| Tetrazole | Product | Name | Yield |
|---|---|---|---|
| 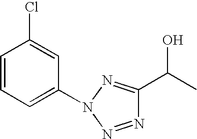 | 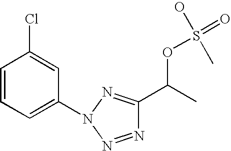 Example 28.1 | Methanesulfonic acid 1-[2-(3-chloro-phenyl)-2H-tetrazol-5-yl]-ethylester | 99.4%670 mg |

| | |
|---|---|
| ¹H NMR | (300 MHz, CDCl₃): δ (ppm) 8.18 (t, 1H), 8.08 (m, 1H), 7.54 (m, 2H), 6.15 (q, 1H), 3.16 (s, 3H), 1.99 (d, 3H) |

| | | | |
|---|---|---|---|
| 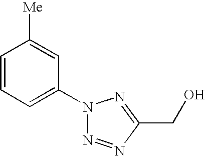 | 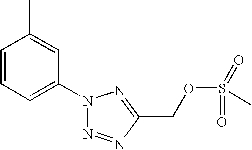 Example 28.2 | Methanesulfonic acid 2-(3-chloro-phenyl)-2H-tetrazol-5-ylmethylester | Quantitative |

| | |
|---|---|
| ¹H NMR | (300 MHz, CDCl₃): δ (ppm) 7.95 (m, 2H), 7.47 (t, 1H), 7.35 (d, 1H), 5.61 (s, 2H), 3.19 (s, 3H), 2.5 (s, 3H) |

| | | | |
|---|---|---|---|
| 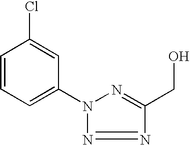 | 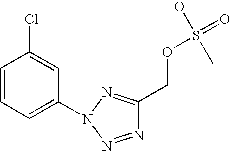 Example 28.3 | Methanesulfonic acid 2-(3-chloro-phenyl)-2H-tetrazol-5-ylmethylester | Quantitative |

| | |
|---|---|
| ¹H NMR | (300 MHz, CDCl₃): δ (ppm) 8.17 (dd, 1H), 8.06 (m, 1H), 7.53 (m, 2H), 5.61 (s, 2H), 3.19 (s, 3H) |

| | | | |
|---|---|---|---|
| 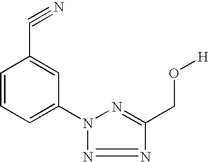 | 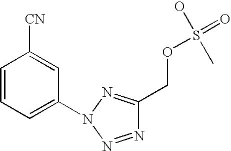 Example 28.4 | Methanesulfonic acid 2-(3-cyano-phenyl)-2H-tetrazol-5-ylmethylester | Quantitative |

| | |
|---|---|
| ¹H NMR | (300 MHz, CDCl₃): δ (ppm) 8.44 (m, 2H), 7.8 (m, 2H), 5.62 (s, 2H), 3.2 (s, 3H) |

### Example 29.1

### Amino-Triazole syntheses: 2-(methylthio)-4,5,6,7-tetrahydro-1H-1,3-diazepine

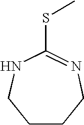

Methyl iodide (0.55 mL, 1.15 mmol) was added to a solution of 1,3-diazepane-2-thione (J. Med. Chem. 1981, 24, 1089) (1.00 g, 7.68 mmol) in acetone (8 mL). The reaction mixture was refluxed for 15 min. EtOH was added to the hot solution to dissolve the solids. After cooling to r.t. hexane was added and the precipitate was collected by filtration, washed with hexane and dried to give 1.79 g (86%) of the crude title compound which was used directly in the next step.

### Example 29.2

### 2-Methylsulfanyl-1,4,5,6-tetrahydro-pyrimidine

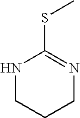

Tetrahydro-pyrimidine-2-thione (45 g, 387 mmol) and iodomethane (48 mL, 774 mmol) were stirred in methanol (100 mL) in a sealed flask at 70° C. overnight. The reaction was diluted with diethyl ether and a precipitate formed which was filtered. The solid was dissolved in sodium hydroxide (30 g) in water (400 mL) and extracted with portions of chloroform. The organic extracts were dried over sodium sulfate, filtered and concentrated to give the title compound (68 g, 98%).

### Example 30

### 1,3-diazepan-2-one hydrazone hydroiodide

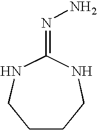

Hydrazine hydrate (0.44 mL, 7.23 mmol) was added to a solution of 2-(methylthio)-4,5,6,7-tetrahydro-1H-1,3-diazepine hydroiodide (1.79 d, 6.58 mmol) in EtOH (12 mL). The reaction mixture was refluxed for 5 h and cooled to r.t. Et₂O was added and the product was collected by filtration, washed with Et₂O and dried under vacuum to give 1.46 g (100%) of the crude title compound which was used directly in the next step.

### Example 31

### 3-pyridin-3-yl-6,7,8,9-tetrahydro-5H-[1,2,4]triazolo[4,3-a][1,3]diazepine

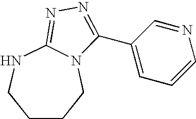

A mixture of 1,3-diazepan-2-one hydrazone hydroiodide (1.00 g, 3.9 mmol) and nicotinoyl chloride hydrochloride (695 mg, 3.9 mmol) was heated in a microwave reactor at 160° C. for 10 min. The reaction mixture was pured into Na₂CO₃ solution, sat., and extracted with DCM. The organic phase was dried and concentrated. Flash chromatography (DCM/MeOH 20:1) gave 1.74 g of the crude title compound which was used directly in the next step.

¹H NMR (300 MHz, CDCl₃): δ (ppm) 8.66 (d, 2H), 7.44 (d, 2H), 3.15 (m, 2H), 3.86 (m, 2H), 1.89 (s, 4H).

### Example 31

### Alternative Synthesis

Nicotinoyl hydrazide (5 g, 36 mmol) was added to a solution of 2-(methylthio)-4,5,6,7-tetrahydro-1H-1,3-diazepine (2.32 g, 30 mmol) in n-BuOH (20 mL). The reaction mixture was heated at 180° C. for 20 min and cooled to r.t. Mixture was the directly subjected to silica gel flash chromatography (EtOAc and 5% MeOH/NH₃) to give 4.95 g of the title compound.

### Example 32

### General Procedure for Formation of Cyclic Triazole Intermediates

The acid chloride was added to a vial followed by pyridine (0.5 mL/mmol). The hydrazine (1 equivalent) was then added to the solution and refluxed at 130° C. over night. The solution was basified using potassium carbonate and aqueous workup was then performed using EtOAc, water, and brine. The organic layer was dried over anhydrous sodium sulfate, filtered and concentrated. An SPE/Flash column was run using a 10-20% MeOH:EtOAc solvent system. The eluting fractions were collected and concentrated. The following table depicts the aminotriazoles formed.

In a similar manner the following compounds were synthesized:

| Example | Structure | Name | Yield |
|---|---|---|---|
| 32.1 | 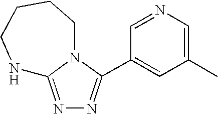 | 3-(5-Methyl-pyridin-3-yl)-5,6,7,8-tetrahydro-4H-1,2,3a,8-tetraaza-azulene | 39% |
| ¹H NMR | (300 MHz, CDCl₃): δ (ppm) 8.48 (m, 2H), 7.73 (m, 1H), 5.87 (NH, 1H), 3.84 (m, 2H), 3.17 | | |
| | (m, 2H), 2.37 (s, 3H), 1.87 (m, 4H) | | |
| 32.2 | 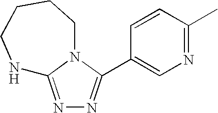 | 3-(6-Methyl-pyridin-3-yl)-5,6,7,8-tetrahydro-4H-1,2,3a,8-tetraaza-azulene | 64% |
| ¹H NMR | (300 MHz, CDCl₃): δ (ppm) 8.60 (m, 1H), 7.79 (m, 1H), 7.25 (d, 1H), 5.42 (NH, 1H), 3.84 | | |
| | (m, 2H), 3.18 (m, 2H), 2.59 (s, 3H), 1.88 (m, 4H) | | |
| 32.3 | 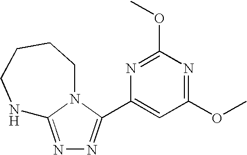 | 3-(2,6-Dimethoxy-pyrimidin-4-yl)-5,6,7,8-tetrahydro-4H-1,2,3a,8-tetraaza-azulene | 16% |
| ¹H NMR | (300 MHz, CDCl₃): δ 7.00 (s, 1H), 4.51 (m, 2H), 4.00 (s, 3H), 3.98 (s, 3H), 3.14 (m, | | |
| | 2H), 1.90 (m, 4H) | | |
| 32.4 | 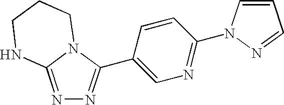 | 3-(6-Pyrazol-1-yl-pyridin-3-yl)-5,6,7,8-tetrahydro-[1,2,4]triazolo[4,3-a]pyrimidine | 27% |
| ¹H NMR | (300 MHz, CDCl₃): δ 8.68 (m, 1H), 8.56 (m, 1H), 8.12 (m, 1H), 8.06 (m, 1H), 7.75 (s, | | |
| | 1H), 6.47 (m, 1H), 6.40 (NH, 1H), 4.06 (t, 2H), 3.49 (t, 2H), 2.10 (m, 2H) | | |
| 32.5 | 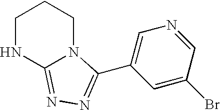 | 3-(5-Bromo-pyridin-3-yl)-5,6,7,8-tetrahydro-[1,2,4]triazolo[4,3-a]pyrimidine | 61% |
| ¹H NMR | (500 MHz, CD₃OD): δ (ppm) 8.84 (d, 1H), 8.76 (d, 1H), 8.33 (t, 1H), 4.13 (t, 2H), 3.41 | | |
| | (dd, 2H), 2.06 (p, 2H) | | |
| 32.6 | 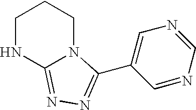 | 3-Pyrimidin-5-yl-5,6,7,8-tetrahydro-[1,2,4]triazolo[4,3-a]pyrimidine | 20% |
| ¹H NMR | (500 MHz, CD₃OD): δ (ppm) 9.23 (s, 1H), 9.11 (s, 2H), 4.42 (m, 2H), 4.16 (t, 2H), | | |
| | 2.08 (p, 2H) | | |
| 32.7 | 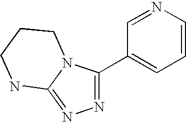 | 3-Pyridin-3-yl-5,6,7,8-tetrahydro-[1,2,4]triazolo[4,3-a]pyrimidine | 52% |
| ¹H NMR | (400 MHz, CD₃OD): 8.85 (m, 1H), 8.63 (m, 1H), 8.12 (m, 1H), 7.57 (m, 1H), 4.10 (t, 2H), | | |
| | 3.40 (t, 2H), 2.05 (m, 2H) | | |
| 32.8 | 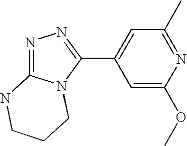 | 3-(2-Methoxy-6-methyl-pyridin-4-yl)-5,6,7,8-tetrahydro-[1,2,4]triazolo[4,3-a]pyrimidine | 80% |
| ¹H NMR | (400 MHz, CDCl₃): δ (ppm) 7.11 (s, 1H), 6.71 (s, 1H), 6.63 (broad s, 1H), 4.06 (t, 2H), | | |
| | 3.93 (s, 3H), 3.47 (m, 2H), 2.46 (s, 3H), 2.06 (m, 2H) | | |
| 32.9 | 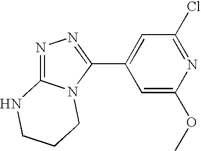 | 3-(2-Chloro-6-methoxy-pyridin-4-yl)-5,6,7,8-tetrahydro-[1,2,4]triazolo[4,3-a]pyrimidine | 36% |

| | |
|---|---|
| ¹H NMR | (300 MHz, CDCl₃): δ (ppm) 7.34 (s, 1H), 6.93 (s, 1H), 5.60 (broad s, 1H), 4.112 (t, 2H), |
| | 3.98 (s, 3H), 3.52 (m, 2H), 2.15 (m, 2H) |

### Example 33

### 3-(2-Methoxy-pyridin-4-yl)-5,6,7,8-tetrahydro-[1,2,4]triazolo[4,3-a]pyrimidine

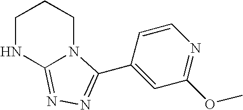

The title compound of Example 32.9 (200 mg) and the palladium on carbon catalyst 10% (100 mg) were combined. The reaction was the flushed with hydrogen gas. EtOH (3.2 mL) and triethylamine (0.6 mL) were also added to the vial. The solution was stirred over night at room temperature. The solution was then filtered through celite. A 10% 1M NH₃ MeOH in DCM silica flash column was run in order to remove any traces of salt. The solution was concentrated and NMR was taken. The solution was concentrated to give a white solid powder (163 mg, 75% yield).

¹H NMR (CDCl₃), δ (ppm): 8.27 (d, 1H), 7.28 (m, 1H), 6.99 (s, 1H), 6.05 (broad s, 1H), 4.14 (t, 2H), 4.1 (s, 3H), 3.6 (t, 2H), 2.1 (m, 2H)

### Example 34

### 5-(5,6,7,8-tetrahydro-[1,2,4]triazolo[4,3-a]pyrimidin-3-yl)-nicotinonitrile

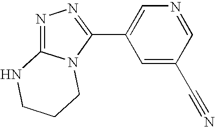

A suspension of the title compound of Example 32.5 (395 mg, 1.4 mmol), NaCN (138 mg, 2.8 mmol) and NiBr₂ (308 mg, 1.4 mmol) in NMP (3 mL) was heated at 200° C. by single-node microwave irradiation for 45 min. After cooling the reaction was diluted with dichloromethane (50 mL) and 13% aqueous ammonia (50 mL) and the layers were separated. The aqueous layer was extracted with six portions of dichloromethane (a total volume of 400 mL). The combined organic layers were dried (sodium sulfate), filtered and concentrated. The residue was purified by reversed phase HPLC eluted with a gradient of acetonitrile in 0.1 M ammonium acetate containing 5% acetonitrile at pH 6.5 to give the title compound (65 mg, 20%) as a solid after freeze-drying.

¹H NMR (400 MHz, CD₃OD): [symbol] (ppm) 9.13 (d, 1H), 8.99 (d, 1H), 8.48 (t, 1H), 4.15 (t, 2H), 3.42 (t, 2H), 2.07 (m, 2H).

### Example 35.1

### 3-Pyridin-3-yl-8-(2-m-tolyl-2H-tetrazol-5-ylmethyl)-5,6,7,8-tetrahydro-[1,2,4]triazolo[4,3-a]pyrimidine

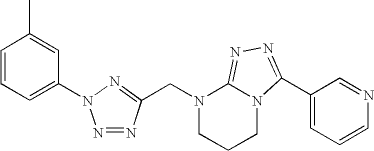

To a screw-cap vial added the title compound of Example 32.7 (60 mg, 0.3 mmol), sodium tert-butoxide (58 mg, 0.6 mmol), N,N-dimethyl formamide (2 mL) and tetrahydrofuran (3 mL). The reaction mixture was heated at 55° C. for 20 min, the solution of the title compound of Example 28.2 in N,N-dimethylformamide (1 mL) was added drop wise to the reaction mixture. The mixture was stirred at 55° C. for 1 hr, and concentrated in vacuo. The residue was diluted in DCM (10 mL), water (10 mL) was added. The aqueous phase was extracted twice with DCM (10 mL), the combined organic phase washed twice with brine (20 mL) dried over anhydrous sodium sulfate and concentrated in vacuo. The crude residue was purified on silica gel using 2 M ammonia in methanol: dichloromethane=5:95, yellow oil was given as product (20.7 mg, 25%).

¹H NMR (300 MHz, CDCl₃): δ (ppm) 8.88 (d, 1H), 8.66 (dd, 1H), 8.04 (dd, 1H), 7.91 (m, 2H), 7.42 (m, 2H), 7.29 (dd, 1H), 5.17 (s, 2H), 4.09 (t, 2H), 3.6 (t, 2H), 2.46 (s, 3H), 2.23 (m, 2H).

In a similar manner the following compounds were synthesized:

| 35.2 | 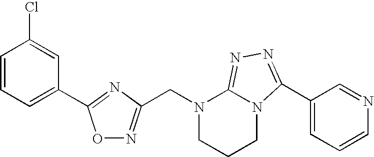 | 8-{[5-(3-chlorophenyl)-1,2,4-oxadiazol-3-yl]methyl}-3-pyridin-3-yl-5,6,7,8-tetrahydro[1,2,4]triazolo[4,3-a]pyrimidine | 22.8 mgYellow oil |
|---|---|---|---|
| ¹H NMR | (300 MHz, CDCl₃): δ (ppm) 8.90 (s, 1H), 8.80 (m, 1H), 8.12 (m, 1H), 8.07 (m, 1H), 8.00 (m, 1H), 7.57 (m, 1H), | | |
| | 7.51 (m, 1H), 7.46 (m, 1H), 5.02 (s, 2H), 4.11 (m, 2H), 3.58 (m, 2H), 2.24 (m, 2H) | | |
| 35.3 | 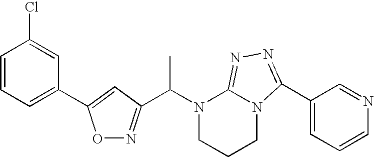 | 8-{1-[5-(3-chlorophenyl)isoxazol-3-yl]ethyl}-3-pyridin-3-yl-5,6,7,8-tetrahydro[1,2,4]triazolo[4,3-a]pyrimidine | 34%Yellow oil |

| | |
|---|---|
| ¹H NMR | (300 MHz, CDCl₃): δ (ppm) 8.90 (m, 1H), 8.68 (m, 1H), 8.08 (m, 1h), 7.74 (m, 1H), 7.62 (m, 1H), 7.40 (m, 3H), |
| | 6.63 (s, 1H), 5.82 (q, 1H), 4.05 (m, 2H), 3.25 (m, 2H), 2.15 (m, 2H), 1.75 (d, 3H) |

| | | | |
|---|---|---|---|
| 35.4 | 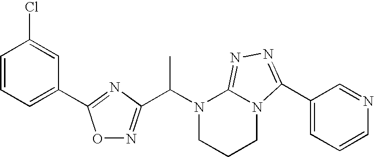 | 8-{1-[5-(3-chlorophenyl)-1,2,4-oxadiazol-3-yl]ethyl}-3-pyridin-3-yl-5,6,7,8-tetrahydro[1,2,4]triazolo[4,3-a]pyrimidine | 20%White solid |

| | |
|---|---|
| ¹H NMR | (300 MHz, CDCl₃): δ (ppm) 8.90 (s, 1H), 8.80 (m, 1H), 8.13 (m, 1H), 8.05 (m, 2H), 7.57 (m, 1H), 7.45 (m, 2H), |
| | 6.00 (q, 1H), 4.08 (m, 2H), 3.50 (m, 2H), 2.20 (m, 2H), 1.75 (d, 3H) |

| | | | |
|---|---|---|---|
| 35.5 | 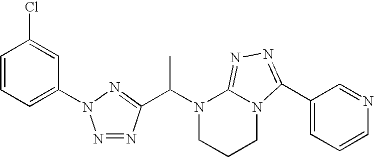 | 8-{1-[2-(3-chlorophenyl)-2H-tetrazol-5-yl]ethyl}-3-pyridin-3-yl-5,6,7,8-tetrahydro[1,2,4]triazolo[4,3-a]pyrimidine | 17%Yellow oil23.1 mg |

| | |
|---|---|
| ¹H NMR | (300 MHz, CDCl₃): δ (ppm) 8.88 (d, 1H), 8.66 (dd, 1H), 8.14 (dd, 1H), 8.04 (m, 2H), 7.42 (m, 3H), 6.16 (q, 1H), |
| | 4.07 (t, 2H), 3.48 (m, 2H), 2.21 (broad s, 2H), 1.83 (d, 3H) |

| | | | |
|---|---|---|---|
| 35.6 | 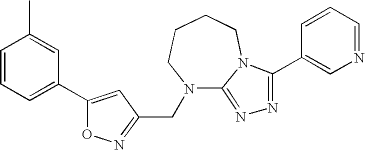 | 3-Pyridin-3-yl-8-(5-m-tolyl-isoxazol-3-ylmethyl)-5,6,7,8-tetrahydro-4H-1,2,3a,8-tetraaza-azulene | 30% |

| | |
|---|---|
| ¹H NMR | (400 MHz, CDCl₃): δ (ppm) 8.75 (m, 1H), 8.67 (m, 1H), 7.94 (m, 1H), 7.55 (m, 2H), 7.42 (m, 1H), 7.29 (t, 1H), |
| | 7.19 (m, 1H), 6.85 (s, 1H), 4.72 (s, 2H), 3.86 (m, 2H), 3.16 (m, 2H), 2.36 (s, 3H), 1.87 (m, 4H) |

| | | | |
|---|---|---|---|
| 35.7 | 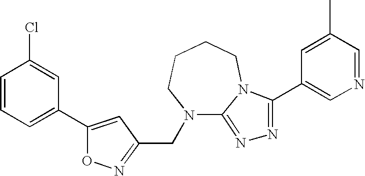 | 8-[5-(3-Chloro-phenyl)-isoxazol-3-ylmethyl]-3-(5-methyl-pyridin-3-yl)-5,6,7,8-tetrahydro-4H-1,2,3a,8-tetraaza-azulene | 73% |

| | |
|---|---|
| ¹H NMR | (400 MHz, CDCl₃): δ (ppm) 8.55 (broad s, 2H), 7.89 (m, 1H), 7.75 (m, 1H), 7.64 (m, 1H), 7.37 (m, 2H), 6.95 (s, |
| | 1H), 4.80 (s, 2H), 3.92 (m, 2H), 3.26 (m, 2H), 2.43 (s, 3H), 1.93 (m, 4H) |

| | | | |
|---|---|---|---|
| 35.8 | 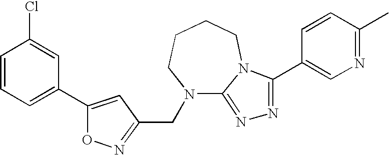 | 8-[5-(3-Chloro-phenyl)-isoxazol-3-ylmethyl]-3-(6-methyl-pyridin-3-yl)-5,6,7,8-tetrahydro-4H-1,2,3a,8-tetraaza-azulene | 78% |

| | |
|---|---|
| ¹H NMR | (400 MHz, CDCl₃): δ (ppm) 8.63 (m, 1H), 7.89 (m, 1H), 7.74 (m, 1H), 7.64 (m, 1H), 7.36 (m, 2H), 7.31 (m, 1H), 6.94 |
| | (s, 1H), 4.75 (s, 2H), 3.87 (m, 2H), 3.18 (m, 2H), 2.63 (s, 3H), 1.88 (m, 4H) |

| | | | |
|---|---|---|---|
| 35.9 | 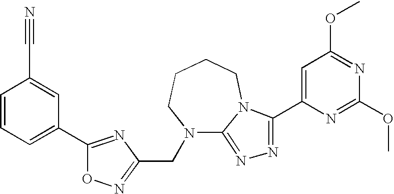 | 3-{3-[3-(2,6-Dimethoxy-pyrimidin-4-yl)-4,5,6,7-tetrahydro-1,2,3a,8-tetraaza-azulen-8-ylmethyl]-[1,2,4]oxadiazol-5-yl}-benzonitrile | 20% |

| | |
|---|---|
| ¹H NMR | (400 MHz, CDCl₃): δ (ppm) 8.40 (m, 1H), 8.34 (m, 1H), 7.86 (m, 1H), 7.66 (t, 1H), 7.24 (s, 1H), 4.94 (s, 2H), 4.64 |
| | (m, 2H), 3.99 (m, 6H), 3.38 (m, 2H), 1.95 (m, 4H) |

| | | | |
|---|---|---|---|
| 35.10 | 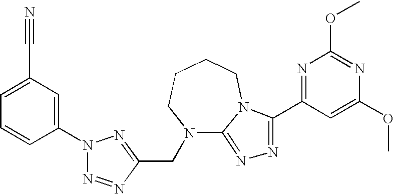 | 3-{5-[3-(2,6-Dimethoxy-pyrimidin-4-yl)-4,5,6,7-tetrahydro-1,2,3a,8-tetraaza-azulen-8-ylmethyl]-tetrazol-2-yl}-benzonitrile | 28% |

| | |
|---|---|
| ¹H NMR | (400 MHz, CDCl₃): δ (ppm) 8.44-8.37 (m, 2H), 7.76 (m, 1H), 7.68 (m, 1H), 7.23 (s, 1H), 5.11 (s, 2H), 4.65 (m, 2H), |
| | 3.99 (m, 6H), 3.41 (m, 2H), 1.94 (m, 4H) |

| | | | |
|---|---|---|---|
| 35.11 | 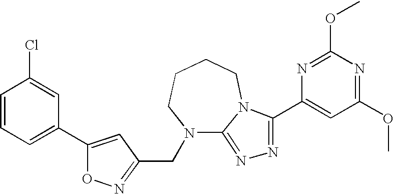 | 8-[5-(3-Chloro-phenyl)-isoxazol-3-ylmethyl]-3-(2,6-dimethoxy-pyrimidin-4-yl)-5,6,7,8-tetrahydro-4H-1,2,3a,8-tetraaza-azulene | 79% |

| | |
|---|---|
| ¹H NMR | (400 MHz, CDCl₃): δ (ppm) 7.75 (m, 1H), 7.64 (m, 1H), 7.37 (m, 2H), 6.95 (s, 1H), 4.77 (s, 2H), 4.59 (m, 2H), 4.00 |
| | (s, 3H), 3.99 (s, 3H), 3.20 (m, 2H), 1.90 (m, 4H) |

| | | | |
|---|---|---|---|
| 35.12 | 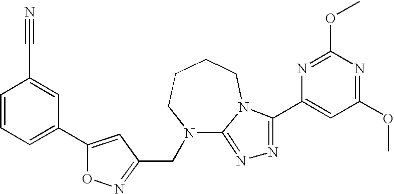 | 3-{3-[3-(2,6-Dimethoxy-pyrimidin-4-yl)-4,5,6,7-tetrahydro-1,2,3a,8-tetraaza-azulen-8-ylmethyl]-isoxazol-5-yl}-benzonitrile | 88% |

| | |
|---|---|
| ¹H NMR | (400 MHz, CDCl₃): δ (ppm) 8.04 (m, 1H), 7.97 (m, 1H), 7.67 (m, 1H), 7.56 (m, 1H), 7.23 (s, 1H), 7.05 (s, 1H), 4.76 |
| | (s, 2H), 4.59 (m, 2H), 3.99 (s, 3H), 3.98 (s, 3H), 3.19 (m, 2H), 1.90 (m, 4H) |

| | | | |
|---|---|---|---|
| 35.13 | 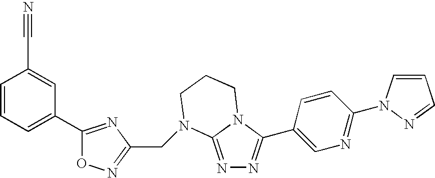 | 3-{3-[3-(6-Pyrazol-1-yl-pyridin-3-yl)-6,7-dihydro-5H-[1,2,4]triazolo[4,3-a]pyrimidin-8-ylmethyl]-[1,2,4]oxadiazol-5-yl}-benzonitrile | 25% |

| | |
|---|---|
| ¹H NMR | (400 MHz, CDCl₃): δ (ppm) 8.68 (d, 1H), 8.55 (d, 1H), 8.39 (s, 1H), 8.32 (d, 1H), 8.15-7.97 (m, 2H), 7.85 (d, 1H), |
| | 7.74 (s, 1H), 7.65 (t, 1H), 6.47 (t, 1H), 4.99 (s, 2H), 4.07 (t, 2H), 3.56 (t, 2H), 2.24 (m, 2H). |

| | | | |
|---|---|---|---|
| 35.14 | 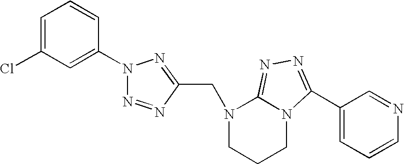 | 8-{[2-(3-chlorophenyl)-2H-tetrazol-5-yl]methyl}-3-pyridin-3-yl-5,6,7,8-tetrahydro[1,2,4]triazolo[4,3-a]pyrimidine | 50%Lightyellow oil |

| | |
|---|---|
| ¹H NMR | (300 MHz, CDCl₃): δ (ppm) 8.88 (s, 1H), 8.66 (d, 1H), 8.15 (s, 1H), 8.04 (m, 2H), 7.42 (m, 3H), 5.18 (s, 2H), 4.1 |
| | (t, 2H), 3.62 (t, 2H), 2.23 (m, 2H) |

| | | | |
|---|---|---|---|
| 35.15 | 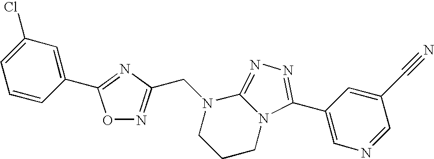 | 5-{8-[5-(3-Chloro-phenyl)-[1,2,4]oxadiazol-3-ylmethyl]-5,6,7,8-tetrahydro-[1,2,4]triazolo[4,3-a]pyrimidin-3-yl}-nicotinonitrile | 45% |

| | |
|---|---|
| ¹H NMR | (500 MHz, CDCl₃): δ (ppm) 9.13 (d, 1H), 8.91 (d, 1H), 8.34 (t, 1H), 8.11 (t, 1H), 8.00 (dt, 1H), 7.57 (ddd, 1H), |
| | 7.47 (t, 1H), 5.02 (s, 2H), 4.14 (t, 2H), 3.60 (dd, 2H), 2.29 (p, 2H) |

| | | | |
|---|---|---|---|
| 35.16 | 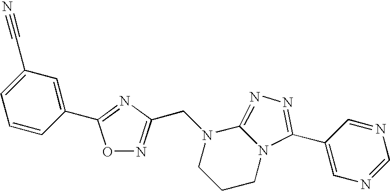 | 3-[3-(3-Pyrimidin-5-yl-6,7-dihydro-5H-[1,2,4]triazolo[4,3-a]pyrimidin-8-ylmethyl)-[1,2,4]oxadiazol-5-yl]-benzonitrile | 75% |

| | |
|---|---|
| ¹H NMR | (500 MHz, CDCl₃): δ (ppm) 9.27 (s, 1H), 9.09 (s, 2H), 8.41 (m, 1H), 8.34 (dt, 1H), 7.88 (dt, 1H), 7.77 (t, 1H), |
| | 5.03 (s, 2H), 4.13 (t, 2H), 3.61 (dd, 2H), 2.29 (m, 2H) |

| | | | |
|---|---|---|---|
| 35.17 | 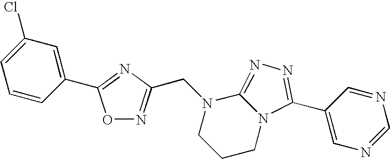 | 8-[5-(3-Chloro-phenyl)-[1,2,4]oxadiazol-3-ylmethyl]-3-pyrimidin-5-yl-5,6,7,8-tetrahydro-[1,2,4]triazolo[4,3-a]pyrimidine | 69% |

| | |
|---|---|
| ¹H NMR | (500 MHz, CDCl₃): δ (ppm) 9.26 (s, 1H), 9.09 (s, 2H), 8.11 (t, 1H), 8.00 (dt, 1H), 7.57 (dddd, 1H), 7.47 (t, 1H), |
| | 5.02 (s, 2H), 4.12 (t, 2H), 3.60 (dd, 2H), 2.28 (m, 2H) |

| | | | |
|---|---|---|---|
| 35.18 | 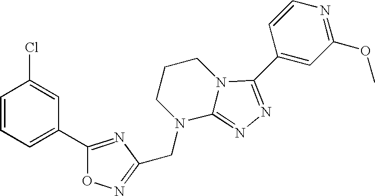 | 8-[5-(3-Chloro-phenyl)-[1,2,4]oxadiazol-3-ylmethyl]-3-(2-methoxy-pyridin-4-yl)-5,6,7,8-tetrahydro-[1,2,4]triazolo[4,3-a]pyrimidine | 85% |

| | |
|---|---|
| ¹H NMR | (400 MHz, CDCl₃): δ (ppm) 8.21 (d, 1H), 8.08 (s, 1H), 7.97 (d, 1H), 7.54 (m, 1H), 7.44 (m, 1H), 7.23 (m, 1H), |
| | 6.95 (s, 1H), 4.98 (s, 2H), 4.09 (t, 2H), 3.94 (s, 3H), 3.53 (t, 2H), 2.20 (m, 2H) |

| | | | |
|---|---|---|---|
| 35.19 | 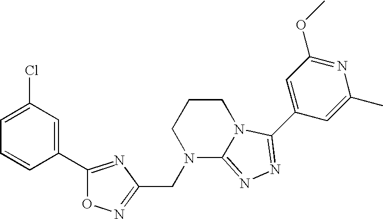 | 8-[5-(3-Chloro-phenyl)-[1,2,4]oxadiazol-3-ylmethyl]-3-(2-methoxy-6-methyl-pyridin-4-yl)-5,6,7,8-tetrahydro-[1,2,4]triazolo[4,3-a]pyrimidine | 82% |

| | |
|---|---|
| ¹H NMR | (400 MHz, CDCl₃): δ (ppm) 8.08 (s, 1H), 7.97 (d, 1H), 7.53 (d, 1H), 7.44 (t, 1H), 7.11 (s, 1H), 6.71 (s, 1H), |
| | 4.98 (s, 2H), 4.08 (t, 2H), 3.92 (s, 3H), 3.52 (t, 2H), 2.47 (s, 3H), 2.19 (m, 2H) |

| | | | |
|---|---|---|---|
| 35.20 | 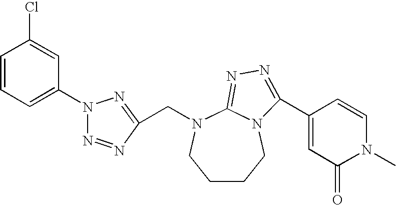 | 40{8-[2-(3-Chloro-phenyl)-2H-tetrazol-5-ylmethyl]-5,6,7,8-tetrahydro-4H-1,2,3a,8-tetraaza-azulen-3-yl}-1-methyl-1H-pyridin-2-one | 27% |

| | |
|---|---|
| ¹H NMR | (400 MHz, CDCl₃): δ (ppm) 8.14 (m, 1H), 8.01 (m, 1H), 7.46 (m, 2H), 7.36 (d, 1H), 6.65 (dd, 1H), 6.57 (d, 1H), |
| | 5.03 (s, 2H), 3.98 (m, 2H), 3.57 (s, 3H), 3.36 (m, 2H), 2.06 (s, 2H), 1.91 (m, 2H) |

| | | | |
|---|---|---|---|
| 35.21 | 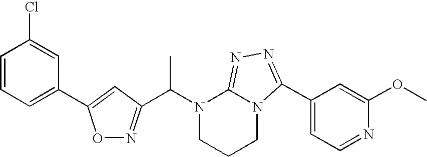 | 8-{1-[5-(3-Chloro-phenyl)-isoxazol-3-yl]-ethyl}-3-(2-methoxy-pyridin-4-yl)-,6,7,8-tetrahydro-[1,2,4]triazolo[4,3-a]pyrimidine | 76%1.33 gOff-whitesolid |

| | |
|---|---|
| ¹H NMR | (300 MHz, CDCl₃): δ (ppm) 8.27 (d, 1H), 7.75 (m, 1H), 7.65 (m, 1H), 7.41 (m, 2H), 7.30 (m, 1H), 6.99 (m, 1H), |
| | 6.62 (s, 1H), 5.87 (q, 1H), 4.09 (m, 2H), 3.99 (s, 3H), 3.43 (m, 1H), 3.27 (m, 1H), 2.10 (m, 2H), 1.75 (m, 3H) |

### Example 36.1

### 4-{8-[5-(3-Chloro-phenyl)-[1,2,4]oxadiazol-3-ylmethyl]-5,6,7,8-tetra hydro-[1,2,4]triazolo[4,3-a]pyrimidin-3-yl-1H-pyridin-2-one

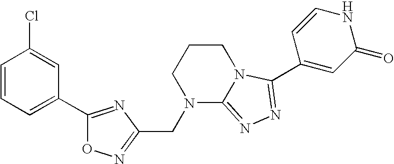

The title compound of Example 35.18 (45 mg, 0.11 mmol) and pyridine hydrochloride (1.0 g, 8.7 mmol) were mixed as solids and heated at 145° C. in an oil bath for 10 min. The reaction mixture was dissolved in water (50 mL) and extracted with DCM (4 times 10 mL). The combined organic layers were concentrated and purified with preparative reversed phase HPLC using a gradient of MeCN in 0.15% TFA in water:MeCN 95:5 to give the title compound (32%).

¹H NMR (400 MHz, CD₃OD): δ (ppm) 8.11 (s, 1H), 8.04 (d, 1H), 7.67 (m, 1H), 7.57 (m, 2H), 6.82 (s, 1H), 6.73 (d, 1H), 4.96 (s, 2H), 4.22 (t, 2H), 3.69 (t, 2H), 2.25 (m, 2H).

In a similar manner the following compound was synthesized:

| Example | Structure | Name | Yield |
|---|---|---|---|
| 36.2 | 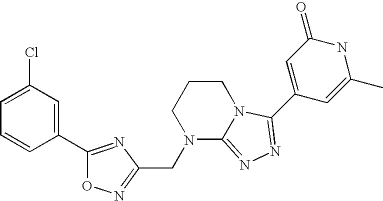 | 4-{8-[5-(3-Chloro-phenyl)-[1,2,4]oxadiazol-3-ylmethyl]-5,6,7,8-tetrahydro-[1,2,4]-triazolo[4,3-a]pyrimidin-3-yl}-6-methyl-1H-pyridin-2-one | 37% |

| | |
|---|---|
| ¹H NMR | (400 MHz, CDCl₃): δ (ppm) 8.08 (m, 2H), 7.80 (d, 1H), 7.68 (m, 1H), 6.40 (s, 1H), 6.36 (s, 1H), 4.91 (s, 2H), |
| | 4.13 (m, 2H), 3.50 (m, 2H), 2.21 (s, 3H), 2.09 (m, 2H) |

### Example 37

### 5-Methyl-2H-pyridazin-3-one

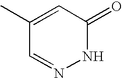

5-Hydroxy-4-methyl-5H-furan-2-one (10.0 g, 87.6 mmol) and hydrazine hydrate (4.38 g, 87.6 mmol) were stirred vigorously at room temperature for 1.5 hours in tetrahydrofuran. A solid began to precipitate and the reaction was heated at 60° C. overnight. The crude reaction mixture was concentrated onto silica gel and purified by column chromatography (0 to 10% methanol in 1:1 EtOAc/dichloromethane) to give 7.7 g (80%) of the title compound.

¹H NMR (300 MHz, CDCl₃): δ (ppm) 11.38 (broad s, 1H), 7.66 (s, 1H), 6.74 (s, 1H), 2.25 (s, 3H).

### Example 38

### 6-Oxo-1,6-dihydro-pyridazine-4-carboxylic acid

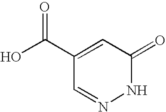

The title compound from Example 37 (0.90 g, 8.2 mmol) was stirred in concentrated sulfuric acid (13 mL) and heated to 45° C. Potassium permanganate (3.6 g, 12 mmol) was added portion wise over 30 min to avoid letting the temperature rise. The reaction was allowed to stir for a further 30 min at 45° C. The reaction was then cooled to room temperature and ice was added to the reaction mixture. The resulting precipitate was collected by vacuum filtration, washing with cold water and diethyl ether to give 0.98 g (87%) of the title compound as the a pale green solid.

¹H NMR (300 MHz, CDCl₃): δ (ppm) 13.39 (broad s, 1H), 8.12 (s, 1H), 7.22 (s, 1H).

### Example 39.1

### 6-Oxo-1,6-dihydro-pyridazine-4-carboxylic acid ethyl ester

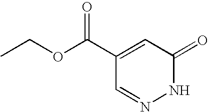

The title compound from Example 38 (1.0 g, 7.13 mmol) was added to a solution of ethanol (16 mL) and acetyl chloride (4 mL) and the resulting suspension was heated to 75° C. and stirred overnight. The reaction mixture was concentrated, diluted with water and extracted with dichloromethane. The organic phase was dried over sodium sulfate, filtered and concentrated to give the title compound.

¹H NMR (300 MHz, CDCl₃): δ (ppm) 10.91 (broad s, 1H), 8.26 (s, 1H), 7.53 (s, 1H), 4.43 (q, 2H), 1.40 (t, 3H).

In a similar manner the following compound was synthesized:

| Example | Structure | Name | Yield |
|---|---|---|---|
| 39.2 | 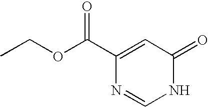 | 6-Oxo-1,6-dihydro-pyrimidine-4-carboxylic acidethyl ester | 52%1.0 g |

| | |
|---|---|
| ¹H NMR | (300 MHz, CDCl₃): δ (ppm) 12.89 (broad s, 1H), 8.36 (s, lH), 7.27 (2H), 4.48 (q, |
| | 2H), 1.41 (t, 3H). |

### Example 40

### 6-Oxo-1,6-dihydro-pyrimidine-4-carboxylic acid

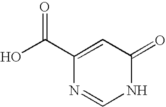

To a solution of sodium hydroxide (1.92 g, 48.1 mmol) in water (100 mL) was added sodium diethyloxalacetate (10.6 g, 50.4 mmol) and formamidine acetate (5.0 g, 48 mmol) and the reaction was allowed to stir overnight at room temperature. The reaction mixture was acidified to pH 2 with hydrochloric acid and then cooled to 0° C. A precipitate formed which was collected by vacuum filtration. The product obtained was the title compound (1.12 g) and was used crude in the next step.

¹H NMR (300 MHz, CDCl₃): δ (ppm) 8.24 (s, 1H), 6.84 (s, 1H).

### Example 41.1

### 6-Oxo-1-(2-trimethylsilanyl-ethoxymethyl)-1,6-dihydro-pyridazine-4-carboxylic acid ethyl ester

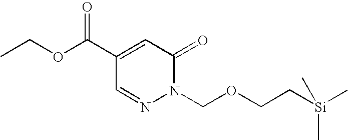

The title compound from Example 39.1 (0.90 g, 5.35 mmol) was stirred in dimethylformamide (20 mL) and diisopropyl ethylamine (1.39 mL, 8.02 mmol) at 0° C. and (2-chloromethoxy-ethyl)-trimethyl-silane (1.88 mL, 10.7 mmol) was added and the reaction was allowed to continue to stir at 0° C. for 2 hours and then overnight at r.t. The reaction mixture was diluted with EtOAc and washed with water and brine. The organic phase was dried over sodium sulfate, filtered and concentrated onto silica gel. The product was purified by column chromatography (0-20% EtOAc/hexanes) to afford the title compound as a clear oil (0.85 g, 53%).

¹H NMR (300 MHz, CDCl₃): δ (ppm) 8.23 (d, 1H), 7.51 (s, 1H), 5.50 (s, 2H), 4.41 (q, 2H), 3.71 (m, 2H), 1.41 (t, 3H), 0.97 (m, 2H), 0.00 (s, 9H).

In a similar manner the following compound was synthesized:

| Example | Structure | Name | Yield |
|---|---|---|---|
| 41.2 | 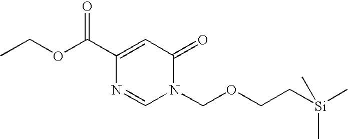 | 6-Oxo-1-(2-trimethylsilanyl-ethoxymethyl)-1,6-dihydro-pyrimidine-4-carboxylicacid ethyl ester | Quant.,0.691 g |

| | |
|---|---|
| ¹H NMR | (300 MHz, CDCl₃): δ (ppm) 8.31 (s, 1H), 7.21 (s, 1H), 5.39 (s, 2H), 4.43 (q, 2H), 3.68 (t, 2H), |
| | 1.41 (t, 3H), 0.95 (m, 2H), 0.00 (s, 9H). |

### Example 42.1

### 6-Oxo-1-(2-trimethylsilanyl-ethoxymethyl)-1,6-dihydro-pyridazine-4-carboxylic acid hydrazide

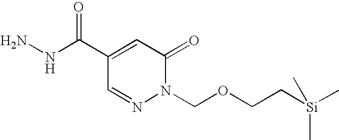

The title compound from Example 41.2 (0.85 g, 2.85 mmol) was stirred in ethanol. Hydrazine hydrate (0.720 g, 14.2 mmol) was added to the solution and the reaction was stirred at 50° C. for 1 hour. The reaction was concentrated and triturated with methanol and diethyl ether to produce a precipitate which was collected by vacuum filtration as the title compound (0.56 g, 57%).

¹H NMR (300 MHz, (CD₃)₂SO): δ (ppm) 10.18 (broad s, 1H), 8.16 (d, 1H), 7.22 (d, 1H), 5.33 (s, 2H), 4.68 (s, 2H), 3.62 (t, 2H), 0.85 (t, 2H), 0.05 (s, 9H).

In a similar manner the following compound was synthesized:

| Example | Structure | Name | Yield |
|---|---|---|---|
| 42.2 | 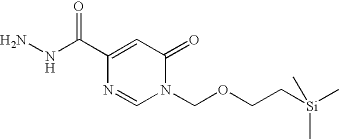 | 6-Oxo-1-(2-trimethylsilanyl-ethoxymethyl)-1,6-dihydro-pyrimidine-4-carboxylic acidhydrazide | 1.42 g |
| ¹H NMR | (300 MHz, CDCl₃): δ (ppm) 8.75 (broad s, 1H), 8.20 (s, 1H), 7.25 (s, 1H), 5.37 (s, 2H), 4.09 (broad s, | | |
| | 2H), 3.68 (m, 2H), 0.97 (m, 2H), 0.01 (s, 9H) | | |
| 42.3 | 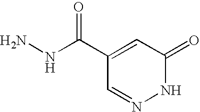 | 6-Oxo-1,6-dihydro-pyridazine-4-carboxylic acidhydrazide | 99% |

| | |
|---|---|
| ¹H NMR | (400 MHz, (CD₃)₂SO): δ 8.05 (d, 1H), 7.09 (d, 1H), 6.40 (broad s, 4H) |

### Example 43.1

### 5-(5,6,7,8-Tetrahydro-[1,2,4]triazolo[4,3-a]pyrimidin-3-yl)-2-(2-trimethylsilanyl-ethoxymethyl)-2H-pyridazin-3-one

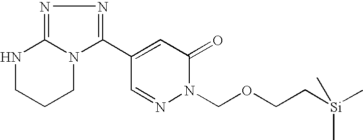

The title compound from Example 29 (0.10 g, 0.768 mmol) and the title compound from Example 42.1 (0.24 g, 0.844 mmol) were combined in a microwave reactor with isopropanol (2 mL) and triethylamine (321 μL, 2.30 mmol) and reacted at 180° C. for 20 min. After cooling to r.t., the reaction mixture was filtered to collect a precipitate and the solid was dissolved in methanol and dichloromethane and concentrated onto silica gel and purified by column chromatography (0-20% methanol in 1:1 EtOAc/dichloromethane) to yield the title compound (0.21 g, 79%).

¹H NMR (300 MHz, DMSO): δ (ppm) 8.38 (s, 1H), 7.38 (s, 1H), 7.02 (s, 1H), 5.34 (s, 2H), 4.16 (t, 2H), 3.65 (t, 2H), 1.91 (m, 3H), 0.87 (3H), −0.04 (s, 9H).

In a similar manner the following compound was synthesized:

| Example | Structure | Name | Yield |
|---|---|---|---|
| 43.2 | 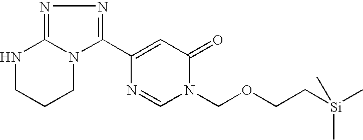 | 6-(5,6,7,8-Tetrahydro-[1,2,4]triazolo[4,3-a]pyrimidin-3-yl)-3-(2-trimethylsilanyl-ethoxymethyl)-3H-pyrimidin-4-one | 0.21 g |

| | |
|---|---|
| ¹H NMR | (300 MHz, CDCl₃): δ (ppm) 8.22 (s, 1H), 7.21 (s, 1H), 6.94 (broad s, 1H), 5.37 (s, 2H), 4.41 (t, |
| | 2H), 3.69 (t, 2H), 3.47 (broad t, 2H), 2.08 (broad quint, 2H), 0.96 (t, 2H), 0.00 (s, 9H). |

### Example 44

### 8-{(R)-1-[5-(3-Chloro-phenyl)-isoxazol-3-yl]-ethyl}-3-(2-methoxy-pyridin-4-yl)-5,6,7,8-tetrahydro-1,2,4]triazolo[4,3-a]pyrimidine

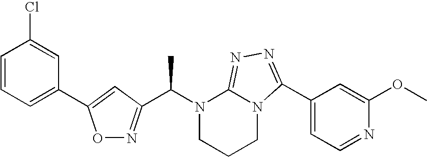

The title compound from Example 35.21 was separated by chiral HPLC using a Chiralpak AS column, eluting with methanol (100%) to give the title compound as a white solid (0.551 g).

¹H NMR (300 MHz, CDCl₃): δ (ppm) 8.27 (d, 1H), 7.75 (m, 1H), 7.65 (m, 1H), 7.41 (m, 2H), 7.30 (m, 1H), 6.99 (m, 1H), 6.62 (s, 1H), 5.87 (q, 1H), 4.09 (m, 2H), 3.99 (s, 3H), 3.43 (m, 1H), 3.27 (m, 1H), 2.10 (m, 2H), 1.75 (m, 3H).

### Example 45

### 2-tert-Butoxycarbonylamino-propionic acid methyl ester

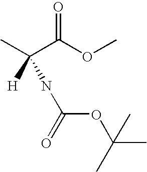

Boc-D-Ala-OH (4.0 g, 21 mmol) and potassium carbonate (11.7 g, 84.6 mmol) was dissolved in dimethylformamide (90 mL) and iodomethane (1.6 mL, 25 mmol) was added to the reaction mixture. The reaction was allowed to stir at room temperature. overnight. The reaction mixture was partitioned between ethyl acetate and water. The organic layer was washed with portions of water and brine, dried over anhydrous sodium sulfate, filtered and concentrated to give the title compound as a colorless oil (3.53 g, 82%).

¹H NMR (300 MHz, CDCl₃): δ (ppm) 5.14 (broad s, 1 h), 4.33 (broad s, 1H), 3.51 (s, 3H), 1.49 (s, 9H).

### Example 46

### (1-Methyl-2-oxo-ethyl)-carbamic acid tert-butyl ester

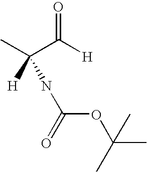

The title compound from Example 45 (3.53 g, 17.4 mmol) was dissolved in toluene (35 mL) at −78° C. and DIBAL-H (26.6 mL, 39.9 mmol) was added dropwise over 1 hour. Methanol (70 mL) was added to the reaction over 10 min. at −78° C. The reaction was moved to an ice bath and 10% w/v citric acid in water (250 mL) was added and the reaction was allowed to stir for 1 hour. The reaction was extracted with portions of ethyl acetate and the organic extracts were washed with water, brine, dried over anhydrous sodium sulfate, filtered and concentrated to give the title compound (2.57 g, 85%) as a white semi-solid.

¹H NMR (300 MHz, CDCl₃): δ (ppm) 9.51 (s, 1H), 5.21 (broad s, 1H), 4.24 (broad s, 1H), 1.53 (s, 9H), 1.35 (d, 3H).

### Example 47

### (2-Hydroxyimino-1-methyl-ethyl)-carbamic acid tert-butyl ester

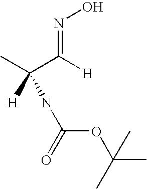

The title compound from Example 46 (2.57 g, 14.8 mmol) was dissolved in methanol (38 mL) and water (38 mL) at 0° C. and sodium carbonate (0.94 g, 8.9 mmol) and hydroxylamine hydrochloride (1.24 g, 17.8 mmol) were added and the reaction was allowed to stir at 0° C. for 30 min. The reaction was then allowed to warm up to r.t. for 4 hours. The reaction mixture was concentrated to half volume and extracted with portions of ethyl acetate. The organic extracts were washed with brine, dried over anhydrous sodium sulfate, filtered and concentrated to give the title compound (2.6 g, 94%) as a white semi-solid which was used further.

### Example 48

### tert-Butyl [(1R,2Z)-2-chloro-2-(hydroxyimino)-1-methylethyl]carbamate

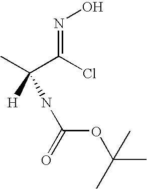

The title compound from Example 47 (2.61 g, 13.9 mmol) was dissolved in dimethylformamide (32 mL) at 40° C. and N-chlorosuccinimide (2.04 g, 15.3 mmol) was added to the reaction in 3 portions. The reaction was heated at 40° C. for 1 hour. The reaction mixture was partitioned between ethyl acetate and water. The organic layer was washed with brine, dried over anhydrous sodium sulfate, filtered and concentrated to give the title compound (2.97 g, 96%) as a colorless oil.

¹H NMR (300 MHz, CDCl₃): δ (ppm) 8.42 (s, 1H), 4.91 (broad s, 1H), 4.69 (broad s, 1H), 1.46 (s, 9H), 1.41 (d, 3H).

### Example 49

### 1-[5-(3-Chloro-phenyl)-isoxazol-3-yl]-ethyl}-carbamic acid tert-butyl Ester

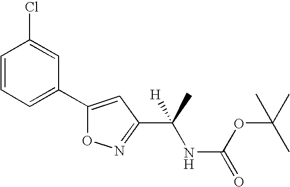

To the title compound from Example 48 (2.97 g, 13.3 mmol) was in dichloromethane (54 mL) was added chlorophenyl acetylene (4.9 mL, 40 mmol)) and triethylamine (3.7 mL, 26.7 mmol) at 0° C. The reaction was allowed to stir at 0° C. for 30 min. before warming up to r.t. overnight. The reaction mixture was concentrated, and then diluted with ethyl acetate. The organic layer washed with 0.1 M hydrochloric acid, sat. sodium bicarbonate solution, water and brine, dried over anhydrous sodium sulfate, filtered and concentrated. The product was purified by column chromatography (20% EtOAc/hexanes) to give the title compound.

¹H NMR (300 MHz, CDCl₃): δ (ppm) 7.81 (s, 1H), 7.76 (m, 1H), 7.65 (m, 2H), 6.51 (s, 1H), 4.98 (broad s, 2H), 1.52 (d, 3H), 1.48 (s, 9H).

### Example 50

### {(1R)-1-[5-(3-Chlorophenyl)isoxazol-3-yl]ethyl}amine

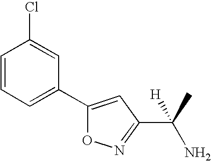

Trifluoroacetic acid (49 mL) was added to a solution of Example 49 (7.93 g, 24.6 mmol) in dichloromethane (94 mL) at 0° C. The resulting mixture was stirred at this temperature for 90 min., and then added to cold saturated NaHCO₃ and the resulting neutralized mixture was extracted with dichloromethane (30 mL). The organic extract washed with brine and dried over magnesium sulfate (anhydrous) and the solvent was removed in vacuo. The residue was then purified by flash column silica gel chromatography with 5% (2 M ammonia methanol) in dichloromethane as eluant giving 4.65 g (85%) of the title compound as a light yellow solid.

¹H NMR (CDCl₃): δ (ppm) 7.71 (s, 1H), 7.66 (m, 1H), 7.43 (m, 2H), 6.56 (s, 1H), 4.31 (q, 1H), 1.65 (broad s, 2H), 1.50 (d,3H).

### Example 51.1

### Acetic acid 1-[2-(3-chloro-phenyl)-2H-tetrazol-5-yl]-ethyl ester

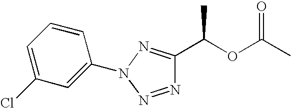

The title compound from Example 18.1 (3.71 g, 16.50 mmol) was dissolved in toluene (90 mL) and Novozyme 435 (0.65 g) was added followed by vinyl acetate (2.3 mL, 24.74 mmol). The reaction was allowed to stir overnight at room temperature. The reaction mixture was filtered, washing with ethyl acetate. The organic phase was concentrated and purified by column chromatography (20-40% EtOAc/hexanes) to give the title compound as an colorless oil (2.13 g).

¹H NMR (300 MHz, CDCl₃): δ (ppm) 8.17 (s, 1H), 8.05 (m, 1 h), 7.50 (m, 2H), 6.29 (q, 1H), 2.16 (s, 3H), 1.79 (d, 3H).

From the same reaction, the following compound was obtained:

| Example | Structure | Name | Yield |
|---|---|---|---|
| 51.2 | 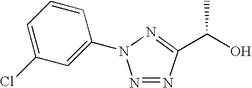 | 1-[2-(3-Chloro-phenyl)-2H-tetrazol-5-yl]-ethanol | 1.68 g,white solid |

| | |
|---|---|
| ¹H NMR | (300 MHz, CDCl₃): δ (ppm) 8.17 (s, 1H), 8.04 (m, 1H), 7.48 (m, 2H), 5.31 (quint, 1H), |
| | 2.71 (d, 1H), 1.76 (d, 3H). |

### Example 52

### 2-{1-[2-(3-Chloro-phenyl)-2H-tetrazol-5-yl]-ethyl}-isoindole-1,3-dione

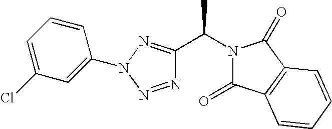

The title compound from Example 51.2 (1.62 g, 7.21 mmol) was combined with phthalimide (2.12 g, 14.4 mmol), triphenyl phosphine (3.80 g, 14.5 mmol) and tetrahydrofuran (50 mL) at room temperature. Diethyl azodicarboxylate (2.28 mL, 14.5 mmol) was added and the reaction was stirred at r.t. overnight. The reaction mixture was partitioned between ethyl acetate and water. The aqueous layer was extracted with ethyl acetate and the combined organics were washed with brine, dried over magnesium sulfate, filtered and concentrated. The product was purified by column chromatography (30% EtOAc/hexanes) to give the title compound as a white solid (2.46 g, 96%).

¹H NMR (300 MHz, CDCl₃): δ (ppm) 8.12 (s, 1H), 7.89 (m, 1H), 7.76 (m, 2H), 7.45 (m, 2H0, 5.87 (q, 1H), 2.06 (d, 3H).

### Example 53

### 1-[2-(3-Chloro-phenyl)-2H-tetrazol-5-yl]-ethylamine

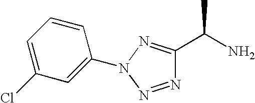

The title compound from Example 52 (2.46 g, 6.95 mmol) was stirred in methanol (50 mL) at 0° C. and hydrazine hydrate (2.0 mL, 41.70 mmol) was added to the solution. The reaction was stirred at 0° C. for 2 hours. Hydrochloric acid (2M, 50 mL) was added to the reaction and it was allowed to stir at room temperature overnight. A white precipitate formed and was filtered and washed with water. The aqueous washings were washed with dichloromethane and basified with aq. potassium carbonate to pH 14 and then extracted with portions of ethyl acetate. The organics were combined and washed with brine, dried over magnesium sulfate, filtered and concentrated to give the title compound as an oil (1.54 g, 99%).

¹H NMR (300 MHz, CDCl₃): δ (ppm) 8.16 (s, 1H), 8.05 (m, 1H), 7.47 (m, 2H), 4.50 (q, 1H), 1.77 (broad s, 2H), 1.64 (d, 3H).

### Example 54

### (3-Oxo-propyl)-carbamic acid tert-butyl ester

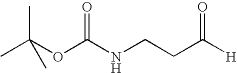

Tert-butyl N-(3-hydroxypropyl)-carbamate (15.38 g, 87.74 mmol) and pyridinium chlorochromate (41.61 g, 193.0 mmol) were stirred in dichloromethane (350 mL) at r.t. overnight. The resulting solution was filtered through a plug of silica, washing with 20% EtOAc/hexanes. The organic was concentrated onto silica gel and purified by column chromatography (40% EtOAc/hexanes) to give the title compound as a colorless oil 6.11 g, 40%).

¹H NMR (300 MHz, CDCl₃): δ (ppm) 3.42 (m, 2H), 2.71 (m, 2H), 1.42 (s, 9H).

### Example 55.1

### (3-{1-[2-(3-Chloro-phenyl)-2H-tetrazol-5-yl]-ethylamino}-propyl)-carbamic acid tert-butyl ester

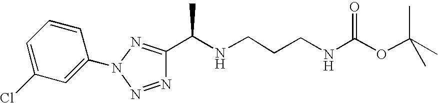

The title compound from Example 53 (2.59 g, 11.60 mmol) and the title compound from Example 54 (3.01 g, 17.4 mmol) were stirred together in dichloromethane (50 mL) at room temperature. To this was slowly added Na(OAc)₃BH (3.69 g, 17.4 mmol) and the reaction was stirred for 2 hours. The reaction was diluted with saturated sodium bicarbonate solution, extracted with portions of dichloromethane, dried over sodium sulfate, filtered and concentrated. The product was purified by column chromatography (5% 2M NH₃ in MeOH/EtOAc) to give the title compound as a colorless oil (3.89 g, 88%).

¹H NMR (300 MHz, CDCl₃): δ (ppm) 8.17 (s, 1H), 8.06 (m, 1H), 7.47 (m, 2H), 5.00 (broad s, 1H), 4.26 (q, 1H), 3.21 (broad s, 2H), 2.65 (t, 2H), 1.68 (m, 3H), 1.59 (d, 3H), 1.42 (s, 9H).

In a similar manner the following compound was synthesized:

| Example | Structure | Name | Yield |
|---|---|---|---|
| 55.2 | 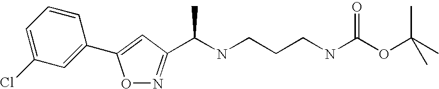 | (3-{1-[5-(3-Chloro-phenyl)-isoxazol-3-yl]-ethylamino}-propyl)-carbamic acid tert-butylester-4-one | 73%,2.89 g |

| | |
|---|---|
| ¹H NMR | (300 MHz, CDCl₃): δ (ppm) 7.77 (s, 1H), 7.68 (m, 1H), 7.41 (m, 2H), 6.55 (s, 1H), 4.97 (broad s, 1H), 4.02 (q, 1H), |
| | 3.21 (broad q, 2H), 2.62 (m, 2H), 1.65 (quint, 2H), 1.44 (m, 12H) |

### Example 56.1

### N*1*-{1-[2-(3-Chloro-phenyl)-2H-tetrazol-5-yl]-ethyl}-propane-1,3-diamine

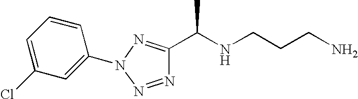

The title compound of Example 55.2 (3.89 g, 10.2 mmol) was dissolved in dichloromethane (50 mL) at 0° C. and trifluoroacetic acid (20 mL) was added dropwise to the reaction. It was allowed to stir at 0° C. for 3 hours before being concentrated and diluted with chloroform (100 mL). The reaction was basified with saturated sodium bicarbonate solution (100 mL) and the aqueous layer was extracted with portions of chloroform. The combined organic extracts were dried over sodium sulfate, filtered and concentrated to give the title compound without further purification (2.87 g, assume 100% yield).

In a similar manner the following compound was synthesized:

| Example | Structure | Name | Yield |
|---|---|---|---|
| 56.2 | 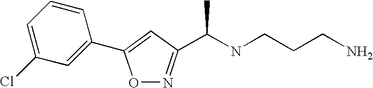 | N*1*-{1-[5-(3-Chloro-phenyl)-isoxazol-3-yl]-ethyl}-propane-1,3-diamine | 100%,2.53 g |

| | |
|---|---|
| ¹H NMR | (300 MHz, CDCl₃): δ (ppm) 7.77 (s, 1H), 7.68 (m, 1H), 7.41 (d, 2H), 6.54 (s, 1H), 4.04 (q, 1H), |
| | 2.82 (t, 2H), 2.66 (m, 2H), 1.65 (quint, 2H), 1.46 (d, 3H). |

### Example 57.1

### 1-{1-[2-(3-Chloro-phenyl)-2H-tetrazol-5-yl]-ethyl}-tetrahydro-pyrimidine-2-thione

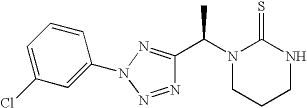

The title compound of Example 56.1 (2.87 g, 10.2 mmol) was dissolved in dichloromethane (50 mL) at −78° C. and thiocarbonyl diimidazole (3.0 g, 15.3 mmol) in dichloromethane (50 mL) was added dropwise. The reaction was allowed to stir at −78° C. for 30 min. and then heated to reflux overnight. The reaction mixture was cooled, washed with water, dried over sodium sulfate, filtered and concentrated onto silica gel. It was purified by column chromatography (40-60% EtOAc/Hexanes) to give the title compound as a white solid (2.26 g, 69%).

¹H NMR (300 MHz, CDCl₃): δ (ppm) 8.15 (s, 1H), 8.05 (m, 1H), 7.48 (m, 2H), 7.29 (q, 1H), 6.77 (s, 1H), 3.35 (m, 4H), 2.09 (m, 2H), 1.77 (d, 3H).

In a similar manner the following compound was synthesized:

| Example | Structure | Name | Yield |
|---|---|---|---|
| 57.2 | 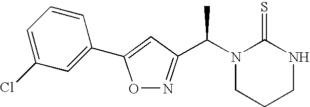 | 1-{1-[5-(3-Chloro-phenyl)-isoxazol-3-yl]-ethyl}-tetrahydro-pyrimidine-2-thione | 82%1.99 g |

| | |
|---|---|
| ¹H NMR | (300 MHz, CDCl₃): δ (ppm) 7.77 (s, 1H), 7.66 (m, 1H), 7.41 (m, 2H), 7.04 (q, 1H), 6.66 (s, |
| | 1H), 6.43 (s, 1H), 3.33 (m, 2H), 3.20 (m, 1H), 1.94 (broad m, 3H), 1.68 (d, 3H). |

### Example 58.1

### 1-{1-[2-(3-Chloro-phenyl)-2H-tetrazol-5-yl]-ethyl}-2-methylsulfanyl-1,4,5,6-tetrahydro-pyrimidine

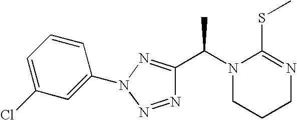

The title compound from Example 57.1 (2.26 g, 7.00 mmol), sodium tert-butoxide (0.672 g, 7.00 mmol) and iodomethane (0.66 mL, 10.50 mmol) in tetrahydrofuran (30 mL) were stirred together at room temperature for 2 hours. The reaction mixture was concentrated and partitioned between ethyl acetate and water. The organic phase washed with brine, dried over sodium sulfate, filtered and concentrated to give the title compound as a yellow oil (2.35 g, quant.).

¹H NMR (300 MHz, CDCl₃): δ (ppm) 8.16 (s, 1H), 8.05 (m, 1H), 7.48 (m, 2H), 5.72 (q, 1H0, 3.51 (m, 2H), 3.30 (m, 1H), 3.12 (m, 1H), 2.38 (s, 3H), 1.85 (m, 2H), 1.74 (s, 3H).

In a similar manner the following compound was synthesized:

| Example | Structure | Name | Yield |
|---|---|---|---|
| 58.2 | 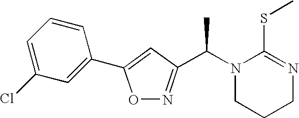 | 1-{1-[5-(3-Chloro-phenyl)-isoxazol-3-yl]-ethyl}-2-methylsulfanyl-1,4,5,6-tetrahydro-pyrimidine | 100%,2.14 g |

| | |
|---|---|
| ¹H NMR | (300 MHz, CDCl₃): δ (ppm) 7.77 (s, 1H), 7.68 (m, 1H), 7.42 (m, 2H), 6.54 (s, 1H), 5.38 (q, |
| | 1H), 3.50 (m, 2H), 3.23 (m, 1H), 2.98 (m, 1H), 2.40 (s, 3H), 1.83 (m, 2H), 1.66 (d, 3H) |

### Example 59.1

### 5-(8-{1-[2-(3-Chloro-phenyl)-2H-tetrazol-5-yl]-ethyl}-5,6,7,8-tetrahydro-[1,2,4]triazolo[4,3-a]pyrimidin-3-yl)-2H-pyridazin-3-one

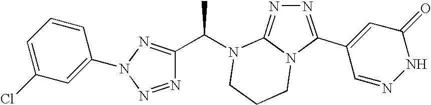

The title compound from Example 58.1 (0.094 g, 0.28 mmol) and the title compound from Example 42.3 (0.077 g, 0.56 mmol) were stirred together in DMSO at 120° C. for 24 hours. The reaction mixture was concentrated and diluted with ethyl acetate and washed with portions of water. The organic layer washed with brine, dried over sodium sulfate, filtered and concentrated onto silica gel. The product was purified by column chromatography (0-8% 2M NH₃ in MeOH/EtOAc) to give the title compound as a pale yellow solid (0.036 g, 41%).

¹H NMR (300 MHz, CDCl₃): δ (ppm) 8.58 (s, 1H), 8.14 (s, 1H), 8.03 (m, 1H), 7.49 (m, 2H), 7.26 (s, 1H), 6.18 (q, 1H), 4.15 (m, 2H), 2.23 9t, 2H), 1.85 (d, 4H).

In a similar manner the following compounds were synthesized:

| Example | Structure | Name | Yield |
|---|---|---|---|
| 59.2 | 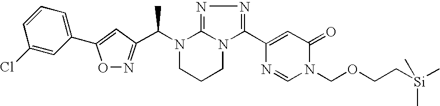 | 6-(8-{1-[5-(3-Chloro-phenyl)-isoxazol-3-yl]-ethyl}-5,6,7,8-tetrahydro-[1,2,4]triazolo[4,3-a]pyrimidin-3-yl)-3-(2-trimethylsilanyl-ethoxymethyl)-3H-pyrimidin-4-one | 100% |
| 59.3 | 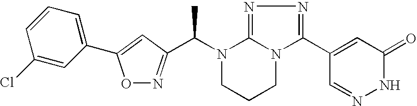 | 5-(8-{1-[5-(3-Chloro-phenyl)-isoxazol-3-yl]-ethyl}-5,6,7,8-tetrahydro-[1,2,4]triazolo[4,3-a]pyrimidin-3-yl)-2H-pyridazin-3-one | 13%0.03 gLightyellowsolid |
| ¹H NMR | (300 MHz, CDCl₃): δ (ppm) 10.79 (s, 1H), 8.59 (s, 1H), 7.74 (s, 1H), 7.63 (m, 1H), 7.41 (m, 2H), 6.94 (s, 1H), | | |
| | 6.57 (s, 1H), 5.89 (q, 1H), 4.13 (m, 2H), 3.48 (m, 1H), 3.34 (m, 1H), 2.17 (m, 2H), 1.77 (d, 3H) | | |
| 59.4 | 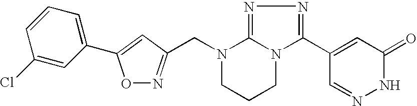 | 5-{8-[5-(3-Chloro-phenyl)-isoxazol-3-ylmethyl]-5,6,7,8-tetrahydro-[1,2,4]triazolo[4,3-a]pyrimidin-3-yl}-2H-pyridazin-3-one | 46%35 mgWhitesolid |
| ¹H NMR | (400 MHz, (CD₃)₂SO): δ (ppm) 13.08 (s, 1H), 8.25 (d, 1H), 7.91 (s, 1H), 7.78 (m, m, 1H), 7.52 (m, 2H), | | |
| | 7.08 (s, 1H), 6.96 (d, 1H), 4.76 (s, 2H), 4.15 (t, 2H), 3.35 (t, t, 2H), 2.04 (m, 2H) | | |

### Example 60

### 6-(8-{(R)-[5-(3-Chloro-phenyl)-isoxazol-3-yl]-ethyl}-5,6,7,8-tetrahydro-[1,2,4]triazolo[4,3-a]pyrimidin-3-yl)-3H-pyrimidin-4-one

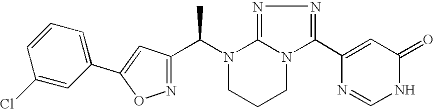

The title compound from Example 59.2 (0.16 g, 0.48 mmol) was dissolved in dichloromethane (2.5 mL) and cooled to 0° C. Dimethyl aluminum chloride (1.0M in hexanes, 1.5 mL) was added and the reaction was stirred at 0° C. for 30 min. and warmed to r.t. for 1 hour. The reaction was quenched with methanol (0.5 mL) citric acid (0.5 g) in water (3 mL). The reaction mixture was extracted with portions of chloroform and the organic extracts were dried over sodium sulfate, filtered and concentrated. The product was purified by column chromatography (2-15% 2M NH₃ in MeOH/dichloromethane) to give the title compound (0.021 g, 10%) as a light yellow solid.

¹H NMR (300 MHz, CDCl₃): δ (ppm) 8.44 (s, 1H), 7.74 (s, 1H), 7.64 (m, 1H), 7.39 (m, 3H), 6.61 (s, 1H), 5.87 (q, 1H), 4.48 (m, 1H), 4.36 (m, 1H), 3.40 (m, 1H), 3.22 (m, 1H), 2.11 (broad s, 2H), 1.75 (d, 3H).

### Example 61.1

### 4-(8-{(R)-1-[5-(3-Chloro-phenyl)-isoxazol-3-yl]-ethyl}-5,6,7,8-tetrahydro-[1,2,4]-triazolo[4,3-a]pyrimidin-3-yl)-1H-pyridin-2-one

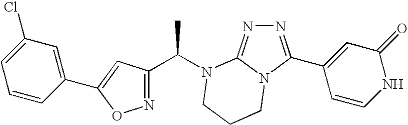

The title compound from Example 44 (0.05 g, 0.114 mmol) was dissolved in acetic acid (1 mL) and hydrogen bromide in ethanol (1 mL) was added. The reaction was heated at 80° C. overnight. The reaction was diluted with water and quenched with aq. sodium carbonate. The aqueous phase was extracted with portions of dichloromethane and the organic extracts were dried over sodium sulfate, filtered and concentrated to give the title compound as a pale solid (0.049 g, 100%).

¹H NMR (300 MHz, CDCl₃): δ (ppm) 7.98 (s, 1H), 7.61 (m, 1H), 7.44 (d, 1H), 7.37 (m, 2H), 6.94 (dt, 1H), 6.68 (s, 1H), 6.59 (s, 1H), 5.85 (q, 1H), 4.09 (m, 3H), 3.42 (m, 1H), 3.26 (m,1H), 2.10 (m, 2H), 1.73 (d, 3H).

In a similar manner the following compound was synthesized:

| Example | Structure | Name | Yield |
|---|---|---|---|
| 61.2 | 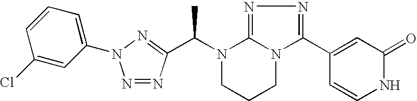 | 4-(8-{(R)-1-[2-(3-Chloro-phenyl)-2H-tetrazol-5-yl]-ethyl}-5,6,7,8-tetrahydro-[1,2,4]triazolo[4,3-a]pyrimidin-3-yl)-1H-pyridin-2-one | 0.067 g,73% |

| | |
|---|---|
| ¹H NMR | (300 MHz, CDCl₃): δ (ppm) 8.14 (d, 1H), 8.03 (m, 1H), 7.47 (m, 3H), 6.97 (dd, 1H), 6.69 (s, 1H), 6.18 (q, |
| | 1H), 4.15 (m, 2H), 3.47 (m, 2H), 2.19 (t, 2H), 1.84 (d, 4H) |

### Example 62

### 4-(8-{(R)-1-[5-(3-Chloro-phenyl)-isoxazol-3-yl]-ethyl}-5,6,7,8-tetrahydro-[1,2,4]triazolo[4,3-a]pyrimidin-3-yl)-1-methyl-1H-pyridin-2-one

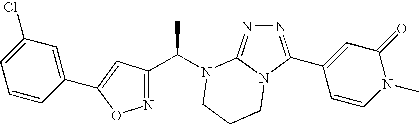

The title compound from Example 61.2 (0.040 g, 0.094 mmol) was dissolved in dimethylformamide (0.5 mL) with sodium hydride (0.005 g, 0.113 mmol) and heated to 50° C. for 1.5 hours. Iodomethane (0.2 g, 0.14 mmol) was then added and the reaction was allowed to stir overnight at 50° C. The reaction was diluted with dichloromethane and washed with portions of water. The organic phase was dried over sodium sulfate, filtered and concentrated and purified by column chromatography (0-10% 2M NH₃ in MeOH/dichloromethane) to give the title compound (0.022 g).

¹H NMR (300 MHz, CDCl₃): δ (ppm) 7.73 (m, 1H), 7.62 (m, 1H), 7.38 (m, 2H), 6.88 (dt, 1H), 6.67 (m, 1H), 6.59 (s, 1H), 5.85 (q, 1H), 4.10 (m, 3H), 3.58 (s, 3H), 3.39 (m, 1H), 3.28 (m, 1H), 2.09 (m, 2H), 1.75 (d, 3H).

### Example 62

### 1-[5-(3-Chloro-phenyl)-isoxazol-3-ylmethyl]-2-methylsulfanyl-1,4,5,6-tetrahydro-pyrimidine

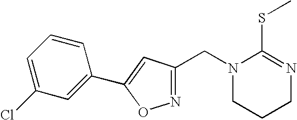

The title compound of Example 9.1 (90 mg, 0.35 mmol) was taken in 2 mL DMF and cooled to 0° C. Sodium hydride (55% in mineral oil) (30 mg, 0.7 mmol) was added to it. The slurry was stirred for 1 h. The title compound of example 29.2 (100 mg, 0.35 mmol) was added to the above slurry in one portion. The mixture was stirred for 1 h at 0° C. Water (15 mL) was added and the product precipitated and was dried under vacuum to yield 45 mg (40%) white solid product.

¹H NMR (400 MHz, CDCl₃): δ (ppm) 7.70 (m, 1H), 7.60 (m, 1H), 7.35 (m, 2H), 6.55 (s, 1H), 4.55 (s, 2H), 3.47 (t, 2H), 3.25 (t, 2H), 2.47 (s, 3H), 1.84 (m, 2H).

### Biological Evaluation

### Functional Assessment of mGluR5 Antagonism in Cell Lines Expressing mGluR5D

The properties of the compounds of the invention can be analyzed using standard assays for pharmacological activity. Examples of glutamate receptor assays are well known in the art as described in for example Aramori et al., Neuron 8:757 (1992), Tanabe et al., Neuron 8:169 (1992), Miller et al., J. Neuroscience 15: 6103 (1995), Balazs, et al., J. Neurochemistry 69:151 (1997). The methodology described in these publications is incorporated herein by reference. Conveniently, the compounds of the invention can be studied by means of an assay (FLIPR) that measures the mobilization of intracellular calcium, [Ca²⁺]ᵢ in cells expressing mGluR5 or another assay (IP3) that measures inositol phosphate turnover.

### FLIPR Assay

Cells expressing human mGluR5d as described in WO97/05252 are seeded at a density of 100,000 cells per well on collagen coated clear bottom 96-well plates with black sides and experiments are done 24 h following seeding. All assays are done in a buffer containing 127 mM NaCl, 5 mM KCl, 2 mM MgCl₂, 0.7 mM NaH₂PO₄, 2 mM CaCl₂, 0.422 mg/ml NaHCO₃, 2.4 mg/ml HEPES, 1.8 mg/ml glucose and 1 mg/ml BSA Fraction IV (pH 7.4). Cell cultures in the 96-well plates are loaded for 60 minutes in the above mentioned buffer containing 4 μM of the acetoxymethyl ester form of the fluorescent calcium indicator fluo-3 (Molecular Probes, Eugene, Oreg.) in 0.01% pluronic acid (a proprietary, non-ionic surfactant polyol—CAS Number 9003-11-6). Following the loading period the fluo-3 buffer is removed and replaced with fresh assay buffer. FLIPR experiments are done using a laser setting of 0.800 W and a 0.4 second CCD camera shutter speed with excitation and emission wavelengths of 488 nm and 562 nm, respectively. Each experiment is initiated with 160 μl of buffer present in each well of the cell plate. A 40 μl addition from the antagonist plate was followed by a 50 μL addition from the agonist plate. A 90 second interval separates the antagonist and agonist additions. The fluorescence signal is sampled 50 times at 1 second intervals followed by 3 samples at 5 second intervals immediately after each of the two additions. Responses are measured as the difference between the peak height of the response to agonist, less the background fluorescence within the sample period. IC₅₀ determinations are made using a linear least squares fitting program.

### IP3 Assay

An additional functional assay for mGluR5d is described in WO97/05252 and is based on phosphatidylinositol turnover. Receptor activation stimulates phospholipase C activity and leads to increased formation of inositol 1,4,5,triphosphate (IP₃).

GHEK stably expressing the human mGluR5d are seeded onto 24 well poly-L-lysine coated plates at 40×10⁴ cells/well in media containing 1 μCi/well [3H] myo-inositol. Cells were incubated overnight (16 h), then washed three times and incubated for 1 h at 37° C. in HEPES buffered saline (146 mM NaCl, 4.2 mM KCl, 0.5 mM MgCl₂, 0.1% glucose, 20 mM HEPES, pH 7.4) supplemented with 1 unit/ml glutamate pyruvate transaminase and 2 mM pyruvate. Cells are washed once in HEPES buffered saline and pre-incubated for 10 min in HEPES buffered saline containing 10 mM LiCl. Compounds are incubated in duplicate at 37° C. for 15 min, then either glutamate (80 μM) or DHPG (30 μM) is added and incubated for an additional 30 min. The reaction is terminated by the addition of 0.5 ml perchloric acid (5%) on ice, with incubation at 4° C. for at least 30 min. Samples are collected in 15 ml polypropylene tubes and inositol phosphates are separated using ion-exchange resin (Dowex AG1-X8 formate form, 200-400 mesh, BIORAD) columns. Inositol phosphate separation was done by first eluting glycero phosphatidyl inositol with 8 ml 30 mM ammonium formate. Next, total inositol phosphates is eluted with 8 ml 700 mM ammonium formate/100 mM formic acid and collected in scintillation vials. This eluate is then mixed with 8 ml of scintillant and [3H] inositol incorporation is determined by scintillation counting. The dpm counts from the duplicate samples are plotted and IC₅₀ determinations are generated using a linear least squares fitting program.

### Abbreviations

### BSA Bovine Serum Albumin

### CCD Charge Coupled Device

### CRC Concentration Response Curve

### DHPG 3,5-dihydroxyphenylglycine

### DPM Disintegrations per Minute

### EDTA Ethylene Diamine Tetraacetic Acid

### FLIPR Fluorometric Imaging Plate reader

### GHEK GLAST-containing Human Embryonic Kidney

### GLAST glutamate/aspartate transporter

### HEPES 4-(2-hydroxyethyl)-1-piperazineethanesulfonic acid (buffer)

### IP₃ inositol triphosphate

Generally, the compounds were active in the assay above with IC₅₀ values less than 10 000 nM. In one aspect of the invention, the IC₅₀ value is less than 1000 nM. In a further aspect of the invention, the IC₅₀ value is less than 100 nM.

### Determination of Brain to Plasma Ratio in Rat

Brain to plasma ratios are estimated in female Sprague Dawley rats. The compound is dissolved in water or another appropriate vehicle. For determination of brain to plasma ratio the compound is administrated as a subcutaneous, or an intravenous bolus injection, or an intravenous infusion, or an oral administration. At a predetermined time point after the administration a blood sample is taken with cardiac puncture. The rat is terminated by cutting the heart open, and the brain is immediately retained. The blood samples are collected in heparinized tubes and centrifuged within 30 minutes, in order to separate the plasma from the blood cells. The plasma is transferred to 96-well plates and stored at −20° C. until analysis. The brains are divided in half, and each half is placed in a pre-tarred tube and stored at −20° C. until analysis. Prior to the analysis, the brain samples are thawed and 3 ml/g brain tissue of distilled water is added to the tubes. The brain samples are sonicated in an ice bath until the samples are homogenized. Both brain and plasma samples are precipitated with acetonitrile. After centrifugation, the supernatant is diluted with 0.2% formic acid. Analysis is performed on a short reversed-phase HPLC column with rapid gradient elution and MSMS detection using a triple quadrupole instrument with electrospray ionisation and Selected Reaction Monitoring (SRM) acquisition. Liquid-liquid extraction may be used as an alternative sample clean-up. The samples are extracted, by shaking, to an organic solvent after addition of a suitable buffer. An aliquot of the organic layer is transferred to a new vial and evaporated to dryness under a stream of nitrogen. After reconstitution of the residuals the samples are ready for injection onto the HPLC column.

Generally, the compounds according to the present invention are peripherally restricted with a drug in brain over drug in plasma ratio in the rat of <0.5. In one embodiment, the ratio is less than 0.15.

### Determination of In Vitro Stability

Rat liver microsomes are prepared from Sprague-Dawley rats liver samples. Human liver microsomes are either prepared from human liver samples or acquired from BD Gentest. The compounds are incubated at 37° C. at a total microsome protein concentration of 0.5 mg/mL in a 0.1 mol/L potassium phosphate buffer at pH 7.4, in the presence of the cofactor, NADPH (1.0 mmol/L). The initial concentration of compound is 1.0 μmol/L. Samples are taken for analysis at 5 time points, 0, 7, 15, 20 and 30 minutes after the start of the incubation. The enzymatic activity in the collected sample is immediately stopped by adding a 3.5 times volume of acetonitrile. The concentration of compound remaining in each of the collected samples is determined by means of LC-MS. The elimination rate constant (k) of the mGluR5 inhibitor is calculated as the slope of the plot of In[mGluR5 inhibitor] against incubation time (minutes). The elimination rate constant is then used to calculate the half-life (T ½) of the mGluR5 inhibitor, which is subsequently used to calculate the intrinsic clearance (CLint) of the mGluR5 inhibitor in liver microsomes as:

CLint.=(ln2×incubation volume)/(T½×protein concentration)=μl/min/mg

### Screening for Compounds Active Against TLESR

Adult Labrador retrievers of both genders, trained to stand in a Pavlov sling, are used. Mucosa-to-skin esophagostomies are formed and the dogs are allowed to recover completely before any experiments are done.

### Motility Measurement

In brief, after fasting for approximately 17 h with free supply of water, a multilumen sleeve/sidehole assembly (Dentsleeve, Adelaide, South Australia) is introduced through the esophagostomy to measure gastric, lower esophageal sphincter (LES) and esophageal pressures. The assembly is perfused with water using a low-compliance manometric perfusion pump (Dentsleeve, Adelaide, South Australia). An air-perfused tube is passed in the oral direction to measure swallows, and an antimony electrode monitored pH, 3 cm above the LES. All signals are amplified and acquired on a personal computer at 10 Hz.

When a baseline measurement free from fasting gastric/LES phase III motor activity has been obtained, placebo (0.9% NaCl) or test compound is administered intravenously (i.v., 0.5 ml/kg) in a foreleg vein. Ten min after i.v. administration, a nutrient meal (10% peptone, 5% D-glucose, 5% Intralipid, pH 3.0) is infused into the stomach through the central lumen of the assembly at 100 ml/min to a final volume of 30 ml/kg. The infusion of the nutrient meal is followed by air infusion at a rate of 500 ml/min until an intragastric pressure of 10±1 mmHg is obtained. The pressure is then maintained at this level throughout the experiment using the infusion pump for further air infusion or for venting air from the stomach. The experimental time from start of nutrient infusion to end of air insufflation is 45 min. The procedure has been validated as a reliable means of triggering TLESRs.

TLESRs is defined as a decrease in lower esophageal sphincter pressure (with reference to intragastric pressure) at a rate of >1 mmHg/s. The relaxation should not be preceded by a pharyngeal signal ≦2s before its onset in which case the relaxation is classified as swallow-induced. The pressure difference between the LES and the stomach should be less than 2 mmHg, and the duration of the complete relaxation longer than 1 s.

### Specimen Results are Shown in the Following Table:

| | FLIPR | Brain/Plasma | hERG | Clint |
|---|---|---|---|---|
| | hmGluR5d | Ratio of compound | IonWorks | (human) |
| Example | (nM) | in Rat | (μM) | (μL/min/mg) |
| 35.1 | 37 | 0.005 | 26 | <12 |
| 35.2 | 52 | 0.05 | | |
| 35.17 | 44 | 0.11 | 22 | 14 |
| 59.3 | 34 | 0.00 | 15 | <12 |

1. A compound of formula I: 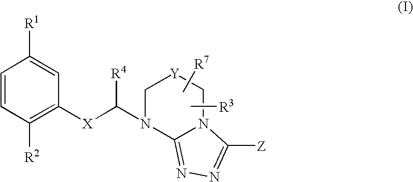
wherein
R¹ is methyl, halogen or cyano;
R² is hydrogen or fluoro;
R³ is hydrogen, fluoro or C₁-C₃ alkyl;
R⁴ is hydrogen or C₁-C₃ alkyl;
Y is C₁-C₂ alkylene;
X is 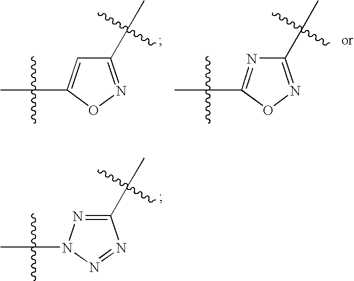
and Z is 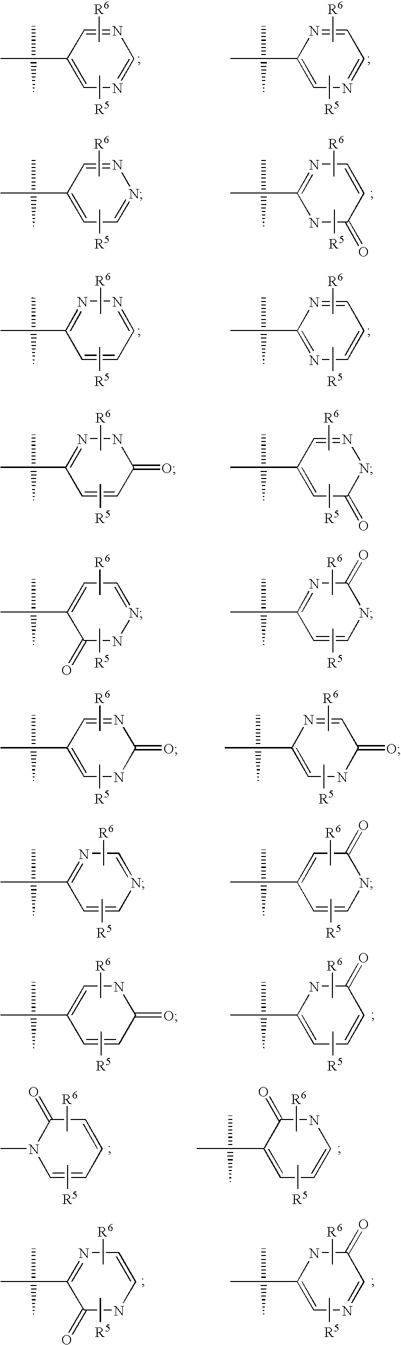 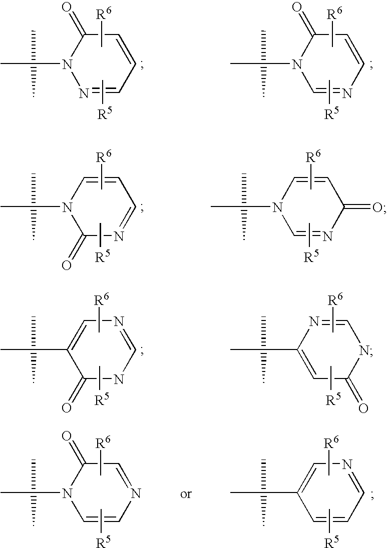
R⁵ is hydrogen, C₁-C₃ alkyl, C₁-C₃ haloalkyl, C₁-C₃ alkoxy, C₁-C₃ haloalkoxy, C₁-C₃ amido alkyl, C₁-C₃ N′alkylamido alkyl, pyrazoyl, C₁-C₃ N′N-dialkylamido alkyl, cyano or C₁-C₃ cyanoalkyl;
R⁶ is hydrogen, C₁-C₃ alkyl, C₁-C₃ haloalkyl, C₁-C₃ alkoxy, C₁-C₃ haloalkoxy, C₁-C₃ amido alkyl, C₁-C₃ N′alkylamido alkyl, C₁-C₃ N′N-dialkylamido alkyl, cyano or C₁-C₃ cyanoalkyl;
R⁷ is hydrogen, fluoro or C₁-C₃ alkyl;
as well as pharmaceutically acceptable salts, hydrates, isoforms, tautomers and/or enantiomers thereof;
with the proviso that the compound of formula I is not
3-{5-[3-(2,6-Dimethoxy-pyrimidin-4-yl)-6,7-dihydro-5H-[1,2,4]triazolo[4,3-a]pyrimidin-8-ylmethyl]-tetrazol-2-yl}-benzonitrile;
8-[2-(3-Chloro-phenyl)-2H-tetrazol-5-ylmethyl]-3-pyridin-3-yl-5,6,7,8-tetrahydro-4H-1,2,3a,8-tetraaza-azulene; or
8-{1-[2-(3-Chloro-phenyl)-2H-tetrazol-5-yl]-ethyl}-3-pyridin-3-yl-5,6,7,8-tetrahydro-[1,2,4]triazolo[4,3-a]pyrimidine.

2. A compound according to claim 1, wherein R⁵ is hydrogen, C₁-C₃ alkyl, C₁-C₃ haloalkyl, C₁-C₃ alkoxy, C₁-C₃ haloalkoxy, C₁-C₃ amido alkyl, C₁-C₃. N′alkylamido alkyl, pyrazoyl, C₁-C₃ N′N-dialkylamido alkyl or C₁-C₃ cyanoalkyl; and R⁶ is hydrogen, C₁-C₃ alkyl, C₁-C₃ haloalkyl, C₁-C₃ alkoxy, C₁-C₃ haloalkoxy, C₁-C₃ amido alkyl, C₁-C₃ N′alkylamido alkyl, C₁-C₃ N′N-dialkylamido alkyl or C₁-C₃ cyanoalkyl.

3. A compound according to claim 1, wherein R¹ is halogen or cyano.

4. A compound according to claim 3, wherein R¹ is chloro.

5. A compound according to claim 3, wherein R¹ is fluoro.

6. A compound according to claim 3, wherein R¹ is methyl.

7. A compound according to claim 3, wherein R¹ is cyano.

8. A compound according to claim 1, wherein R² is hydrogen.

9. A compound according to claim 1, wherein R³ is hydrogen or fluoro.

10. A compound according to claim 1, wherein R⁴ is hydrogen or methyl.

11. A compound according to claim 1, wherein R⁵ is hydrogen, C₁-C₂ alkyl or C₁-C₂ alkoxy.

12. A compound according to claim 1, wherein R⁶ is hydrogen, C₁-C₂ alkyl or C₁-C₂ alkoxy.

13. A compound according to claim 1, wherein R⁷ is C₁-C₂ alkyl or C₁-C₂ alkoxy.

14. A compound according to claim 1, wherein Y is methylene.

15. A compound according to claim 1, wherein Y is ethylene.

16. A compound according to claim 1, wherein Z is

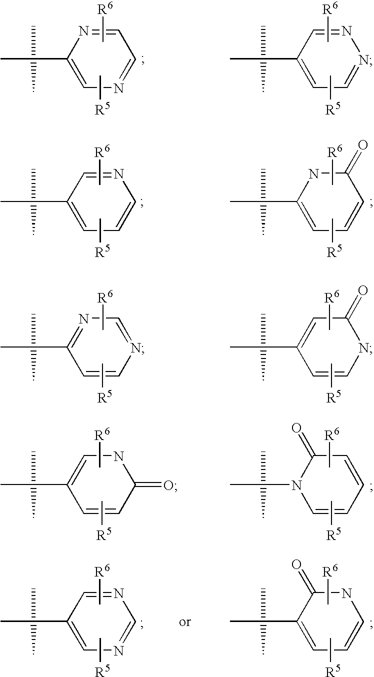

17. A compound according to claim 16, wherein Z is

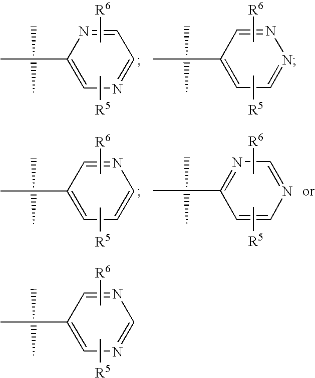

18. A compound according to claim 16, wherein Z is

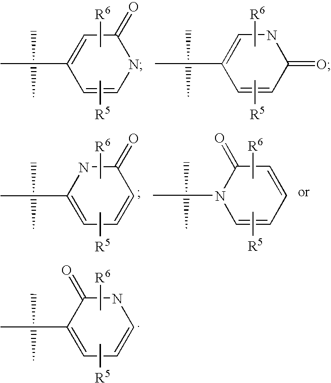

19. A compound selected from
3-Pyridin-3-yl-8-(2-m-tolyl-2H-tetrazol-5-ylmethyl)-5,6,7,8-tetrahydro-[1,2,4]triazolo[4,3a]pyrimidine;
8-{[5-(3-chlorophenyl)-1,2,4-oxadiazol-3-yl]methyl}-3-pyridin-3-yl-5,6,7,8-tetrahydro[1,2,4]triazolo[4,3-a]pyrimidine;
8-{1-[5-(3-chlorophenyl) isoxazol-3-yl]ethyl}-3-pyridin-3-yl-5,6,7,8-tetrahydro[1,2,4]triazolo[4,3-a]pyrimidine;
8-{1-[5-(3-chlorophenyl)-1,2,4-oxadiazol-3-yl]ethyl}-3-pyridin-3-yl-5,6,7,8-tetrahydro[1,2,4]triazolo[4,3-a]pyrimidine;
8-{1-[2-(3-chlorophenyl)-2H-tetrazol-5-yl]ethyl}-3-pyridin-3-yl-5,6,7,8-tetrahydro[1,2,4]triazolo[4,3-a]pyrimidine;
3-Pyridin-3-yl-8-(5-m-tolyl-isoxazol-3-ylmethyl)-5,6,7,8-tetrahydro-4H-1,2,3a,8-tetraaza-azulene;
8-[5-(3-Chloro-phenyl)-isoxazol-3-ylmethyl]-3-(5-methyl-pyridin-3-yl)-5,6,7,8-tetrahydro-4H-1,2,3a,8-tetraaza-azulene;
8-[5-(3-Chloro-phenyl)-isoxazol-3-ylmethyl]-3-(6-methyl-pyridin-3-yl)-5,6,7,8-tetrahydro-4H-1,2,3a,8-tetraaza-azulene;
3-{3-[3-(2,6-Dimethoxy-pyrimidin-4-yl)-4,5,6,7-tetrahydro-1,2,3a,8-tetraaza-azulen-8-ylmethyl]-[1,2,4]oxadiazol-5-yl}-benzonitrile;
3-{5-[3-(2,6-Dimethoxy-pyrimidin-4-yl)-4,5,6,7-tetrahydro-1,2,3a,8-tetraaza-azulen-8-ylmethyl]-tetrazol-2-yl}-benzonitrile;
8-[5-(3-Chloro-phenyl)-isoxazol-3-ylmethyl]-3-(2,6-dimethoxy-pyrimidin-4-yl)-5,6,7,8-tetrahydro-4H-1,2,3a,8-tetraaza-azulene;
3-{3-[3-(2,6-Dimethoxy-pyrimidin-4-yl)-4,5,6,7-tetrahydro-1,2,3a,8-tetraaza-azulen-8-ylmethyl]-isoxazol-5-yl}-benzonitrile;
3-{3-[3-(6-Pyrazol-1-yl-pyridin-3-yl)-6,7-dihydro-5H-[1,2,4]triazolo[4,3-a]pyrimidin-8-ylmethyl]-[1,2,4]oxadiazol-5-yl}-benzonitrile;
8-{[2-(3-chlorophenyl)-2H-tetrazol-5-yl]methyl}-3-pyridin-3-yl-5,6,7,8-tetrahydro[1,2,4]triazolo[4,3-a]pyrimidine;
5-{8-[5-(3-Chloro-phenyl)-[1,2,4]oxadiazol-3-ylmethyl]-5,6,7,8-tetrahydro-[1,2,4]triazolo[4,3-a]pyrimidin-3-yl}-nicotinonitrile;
3-[3-(3-Pyrimidin-5-yl-6,7-dihydro-5H-[1,2,4]triazolo[4,3-a]pyrimidin-8-ylmethyl)-[1,2,4]oxadiazol-5-yl]-benzonitrile;
8-[5-(3-Chloro-phenyl)-[1,2,4]oxadiazol-3-ylmethyl]-3-pyrimidin-5-yl-5,6,7,8-tetrahydro-[1,2,4]triazolo[4,3-a]pyrimidine;
4-{8-[2-(3-Chloro-phenyl)-2H-tetrazol-5-ylmethyl]-5,6,7,8-tetrahydro-4H-1,2,3a,8-tetraaza-azulen-3-yl}-1-methyl-1H-pyridin-2-one;
4-{8-[5-(3-Chloro-phenyl)-[1,2,4]oxadiazol-3-ylmethyl]-5,6,7,8-tetra hydro-[1,2,4]triazolo[4,3-a]pyrimidin-3-yl}-1H-pyridin-2-one;
4-{8-[5-(3-Chloro-phenyl)-[1,2,4]oxadiazol-3-ylmethyl]-5,6,7,8-tetrahydro-[1,2,4]-triazolo[4,3-a]pyrimidin-3-yl}-6-methyl-1H-pyridin-2-one;
5-(8-{1-[2-(3-Chloro-phenyl)-2H-tetrazol-5-yl]-ethyl}-5,6,7,8-tetrahydro-[1,2,4]triazolo[4,3-a]pyrimidin-3-yl)-2H-pyridazin-3-one;
5-(8-{1-[5-(3-Chloro-phenyl)-isoxazol-3-yl]-ethyl}-5,6,7,8-tetrahydro-[1,2,4]triazolo[4,3-a]pyrimidin-3-yl)-2H-pyridazin-3-one;
5-{8-[5-(3-Chloro-phenyl)-isoxazol-3-ylmethyl]-5,6,7,8-tetrahydro-[1,2,4]triazolo[4,3-a]pyrimidin-3-yl}-2H-pyridazin-3-one;
6-(8-{(R)-1-[5-(3-Chloro-phenyl)-isoxazol-3-yl]-ethyl}-5,6,7,8-tetrahydro-[1,2,4]triazolo[4,3-a]pyrimidin-3-yl)-3H-pyrimidin-4-one;
4-(8-{(R)-1-[2-(3-Chloro-phenyl)-2H-tetrazol-5-yl]-ethyl}-5,6,7,8-tetrahydro-[1,2,4]triazolo[4,3-a]pyrimidin-3-yl)-1H-pyridin-2-one;
4-(8-{(R)-1-[5-(3-Chloro-phenyl)-isoxazol-3-yl]-ethyl}-5,6,7,8-tetrahydro-[1,2,4]triazolo[4,3-a]pyrimidin-3-yl)-1-methyl-1H-pyridin-2-one; and
4-(8-{(R)-1-[5-(3-Chloro-phenyl)-isoxazol-3-yl]-ethyl}-5,6,7,8-tetrahydro-[1,2,4]triazolo[4,3-a]pyrimidin-3-yl)-1H-pyridin-2-one
as well as pharmaceutically acceptable salts, hydrates, isoforms, tautomers and/or enantiomers thereof.

20. A compound according to claim 1 for use in therapy.

21. A pharmaceutical composition comprising a compound according to claim 1 as an active ingredient, together with a pharmacologically and pharmaceutically acceptable carrier.

22. Use of a compound according to claim 1, or a pharmaceutically acceptable salt or an optical isomer thereof, for the manufacture of a medicament for the inhibition of transient lower esophageal sphincter relaxations.

23. Use of a compound according to claim 1, or a pharmaceutically acceptable salt or an optical isomer thereof, for the manufacture of a medicament for treatment or prevention of gastroesophageal reflux disease.

24. Use of a compound according to claim 1, or a pharmaceutically acceptable salt or an optical isomer thereof, for the manufacture of a medicament for treatment or prevention of pain.

25. Use of a compound according to claim 1, or a pharmaceutically acceptable salt or an optical isomer thereof, for the manufacture of a medicament for treatment or prevention of anxiety.

26. Use of a compound according to claim 1, or a pharmaceutically acceptable salt or an optical isomer thereof, for the manufacture of a medicament for treatment or prevention of irritable bowel syndrome (IBS).

27. A method for the inhibition of transient lower esophageal sphincter relaxations whereby an effective amount of a compound according to claim 1 is administered to a subject in need of such inhibition.

28. A method for the treatment or prevention of gastroesophageal reflux disease, whereby an effective amount of a compound according to claim 1 is administered to a subject in need of such treatment or prevention.

29. A method for the treatment or prevention of pain, whereby an effective amount of a compound according to claim 1 is administered to a subject in need of such treatment or prevention.

30. A method for the treatment or prevention of anxiety, whereby an effective amount of a compound according to claim 1 is administered to a subject in need of such treatment or prevention.

31. A method for the treatment or prevention of irritable bowel syndrome (IBS), whereby an effective amount of a compound according to claim 1 is administered to a subject in need of such treatment or prevention.

32. A combination comprising (i) at least one compound according to claim 1 and (ii) at least one acid secretion inhibiting agent.

33. A combination according to claim 32 wherein the acid secretion inhibiting agent is selected from cimetidine, ranitidine, omeprazole, esomeprazole, lansoprazole, pantoprazole, rabeprazole or leminoprazole.

34. A compound selected from
5-(3-Methyl-phenyl)-isoxazole-3-carboxylic acid ethyl ester;
[5-(3-Methyl-phenyl)-isoxazol-3-yl]-methanol;
Methanesulfonic acid 5-(3-Methyl-phenyl)-isoxazol-3-ylmethyl ester;
3-(3-Hydroxymethyl-isoxazol-5-yl)-benzonitrile;
Methanesulfonic acid 5-(3-cyano-phenyl)-isoxazol-3-ylmethyl ester;
3-(5-Methyl-pyridin-3-yl)-5,6,7,8-tetrahydro-4H-1,2,3a,8-tetraaza-azulene;
3-(6-Methyl-pyridin-3-yl)-5,6,7,8-tetrahydro-4H-1,2,3a,8-tetraaza-azulene;
3-(2,6-Dimethoxy-pyrimidin-4-yl)-5,6,7,8-tetrahydro-4H-1,2,3a,8-tetraaza-azulene;
3-(6-Pyrazol-1-yl-pyridin-3-yl)-5,6,7,8-tetrahydro-[1,2,4]triazolo[4,3-a]pyrimidine;
3-(5-Bromo-pyridin-3-yl)-5,6,7,8-tetrahydro-[1,2,4]triazolo[4,3-a]pyrimidine;
3-Pyrimidin-5-yl-5,6,7,8-tetrahydro-[1,2,4]triazolo[4,3-a]pyrimidine;
3-(2-Methoxy-6-methyl-pyridin-4-yl)-5,6,7,8-tetrahydro-[1,2,4]triazolo[4,3-a]pyrimidine;
1-[2-(3-Chloro-phenyl)-2H-tetrazol-5-yl]-ethanone;
5-(5,6,7,8-Tetrahydro-[1,2,4]triazolo[4,3-a]pyrimidin-3-yl)-nicotinonitrile;
8-[5-(3-Chloro-phenyl)-[1,2,4]oxadiazol-3-ylmethyl]-3-(2-methoxy-pyridin-4-yl)-5,6,7,8-tetrahydro-[1,2,4]triazolo[4,3-a]pyrimidine;
8-[5-(3-Chloro-phenyl)-[1,2,4]oxadiazol-3-ylmethyl]-3-(2-methoxy-6-methyl-pyridin-4-yl)-5,6,7,8-tetrahydro-[1,2,4]triazolo[4,3-a]pyrimidine;
6-Oxo-1-(2-trimethylsilanyl-ethoxymethyl)-1,6-dihydro-pyridazine-4-carboxylic acid ethyl ester;
6-Oxo-1-(2-trimethylsilanyl-ethoxymethyl)-1,6-dihydro-pyrimidine-4-carboxylic acid ethyl ester;
6-Oxo-1-(2-trimethylsilanyl-ethoxymethyl)-1,6-dihydro-pyridazine-4-carboxylic acid hydrazide;
6-Oxo-1-(2-trimethylsilanyl-ethoxymethyl)-1,6-dihydro-pyrimidine-4-carboxylic acid hydrazide;
5-(5,6,7,8-Tetrahydro-[1,2,4]triazolo[4,3-a]pyrimidin-3-yl)-2-(2-trimethylsilanyl-ethoxymethyl)-2H-pyridazin-3-one;
6-(5,6,7,8-Tetrahydro-[1,2,4]triazolo[4,3-a]pyrimidin-3-yl)-3-(2-trimethylsilanyl-ethoxymethyl)-3H-pyrimidin-4-one;
8-{(R)-1-[5-(3-Chloro-phenyl)-isoxazol-3-yl]-ethyl}-3-(2-methoxy-pyridin-4-yl)-5,6,7,8-tetrahydro-[1,2,4]triazolo[4,3-a]pyrimidine;
{1-[5-(3-Chloro-phenyl)-isoxazol-3-yl]-ethyl}-carbamic acid tert-butyl ester;
{(1R)-1-[5-(3-Chlorophenyl)isoxazol-3-yl]ethyl}amine;
2-{1-[2-(3-Chloro-phenyl)-2H-tetrazol-5-yl]-ethyl}-isoindole-1,3-dione;
1-[2-(3-Chloro-phenyl)-2H-tetrazol-5-yl]-ethylamine;
(3-{1-[2-(3-Chloro-phenyl)-2H-tetrazol-5-yl]-ethylamino}-propyl)-carbamic acid tert-butyl ester;
(3-{1-[5-(3-Chloro-phenyl)-isoxazol-3-yl]-ethylamino}-propyl)-carbamic acid tert-butyl ester-4-one;
N*1*-{1-[2-(3-Chloro-phenyl)-2H-tetrazol-5-yl]-ethyl}-propane-1,3-diamine;
N*1*-{1-[5-(3-Chloro-phenyl)-isoxazol-3-yl]-ethyl}-propane-1,3-diamine;
1-{1-[2-(3-Chloro-phenyl)-2H-tetrazol-5-yl]-ethyl}-tetrahydro-pyrimidine-2-thione;
1-{1-[5-(3-Chloro-phenyl)-isoxazol-3-yl]-ethyl}-tetrahydro-pyrimidine-2-thione;
1-{1-[2-(3-Chloro-phenyl)-2H-tetrazol-5-yl]-ethyl}-2-methylsulfanyl-1,4,5,6-tetrahydro-pyrimidine;
1-{1-[5-(3-Chloro-phenyl)-isoxazol-3-yl]-ethyl}-2-methylsulfanyl-1,4,5,6-tetrahydro-pyrimidine;
6-(8-{1-[5-(3-Chloro-phenyl)-isoxazol-3-yl]-ethyl}-5,6,7,8-tetrahydro-[1,2,4]triazolo[4,3-a]pyrimidin-3-yl)-3-(2-trimethylsilanyl-ethoxymethyl)-3H-pyrimidin-4-one;
1-[5-(3-Chloro-phenyl)-isoxazol-3-ylmethyl]-2-methylsulfanyl-1,4,5,6-tetrahydro-pyrimidine; and
8-{1-[5-(3-Chloro-phenyl)-isoxazol-3-yl]-ethyl}-3-(2-methoxy-pyridin-4-yl)-6,7,8-tetrahydro-[1,2,4]triazolo[4,3-a]pyrimidine.
